(19) United States
(12) Patent Application Publication
(10) Pub. No.: US 2023/0277533 A1
(43) Pub. Date: Sep. 7, 2023

(54) THERAPEUTIC COMBINATIONS COMPRISING UBIQUITIN-SPECIFIC-PROCESSING PROTEASE 1 (USP1) INHIBITORS AND POLY (ADP-RIBOSE) POLYMERASE (PARP) INHIBITORS

(71) Applicant: KSQ Therapeutics, Inc., Cambridge, MA (US)

(72) Inventors: Andrew Alistair WYLIE, Newton, MA (US); Solomon Martin SHENKER, Belmont, MA (US); Pamela Jean SULLIVAN, Dedham, MA (US); Frank STEGMEIER, Acton, MA (US); Anne Louise CADZOW, Boston, MA (US); Hanlan LIU, Lexington, MA (US); Kerstin Wolf SINKEVICIUS, Lincoln, MA (US)

(21) Appl. No.: 17/904,159

(22) PCT Filed: Feb. 12, 2021
(86) PCT No.: PCT/US2021/017912

§ 371 Date: Aug. 12, 2022

Related U.S. Application Data
(60) Provisional application No. 63/146,937, filed on Feb. 8, 2021.
(60) Provisional application No. 63/032,245, filed on May 29, 2020.
(60) Provisional application No. 62/976,864, filed on Feb. 14, 2020.

(51) Int. Cl. A61K 31/502; A61K 31/454; A61K 31/519; A61K 31/55; A61K 31/5025; A61P 35/00

(52) U.S. Cl. CPC A61K 31/502; A61K 31/454; A61K 31/519; A61K 31/55; A61K 31/5025; A61P 35/00

(57) ABSTRACT

The present disclosure provides therapeutic combinations comprising (i) a ubiquitin-specific-processing protease 1 (USP1) inhibitor and (ii) a poly ADP-ribose polymerase (PARP) inhibitor, or a pharmaceutically acceptable salt, hydrate, solvate, amorphous solid, or polymorph thereof, wherein the USP1 inhibitor comprises (I), (II), or (III), or pharmaceutically acceptable salts, hydrates, solvates, amorphous solids, or polymorphs thereof. The present disclosure is also directed to the use of the combinations to inhibit a USP1 and/or PARP protein and/or to treat a disorder responsive to the inhibition of USP1 and/or PARP proteins and USP1 and/or PARP activity. The combinations of the present disclosure are especially useful for treating cancer.

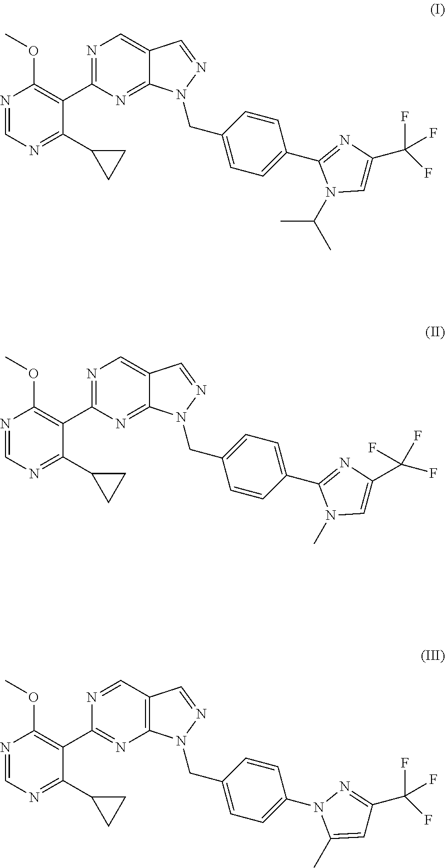 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1 ; COc1ncnc(C2CC2)c1-c1ncc2cnn(Cc3ccc(-n4nc(C(F)(F)F)cc4C)cc3)c2n1]

### CROSS REFERENCE TO RELATED APPLICATIONS

This application claims the benefit of U.S. Provisional Application Nos. 63/146,937, filed Feb. 8, 2021, 63/032,245, filed May 29, 2020, and 62/976,864, filed Feb. 14, 2020, each of which is hereby incorporated by reference in its entirety

### REFERENCE TO A SEQUENCE LISTING SUBMITTED ELECTRONICALLY VIA EFS-WEB

The content of the electronically submitted sequence listing (Name 4195_012PC03_Seglisting_ST25.txt; Size: 7,446 bytes; and Date of Creation: Feb. 11, 2021) is incorporated herein by reference in its entirety.

### BACKGROUND OF THE DISCLOSURE

### Field

The present disclosure provides therapeutic combinations of ubiquitin-specific-processing protease 1 (USP1) inhibitors and Poly (ADP-ribose) polymerase (PARP) inhibitors. Methods of treating cancers comprising administering the combinations are also provided.

### Background

Ubiquitin is a small (76 amino acid) protein that is post-transcriptionally attached to target proteins. The consequence of ubiquitination is determined by the number and linkage topology of ubiquitin molecules conjugated to the target protein. For example, proteins exhibiting lysine 48-linked poly-ubiquitin chains are generally targeted to the proteasome for degradation, while mono-ubiquitination or poly-ubiquitin chains linked through other lysines regulate non-proteolytic functions, such as cell cycle regulation, DNA damage repair, transcription, and endocytosis. Ubiquitination is a reversible process, and enzymes called deubiquitinases remove ubiquitin from target proteins.

USP1 is a deubiquitinase that plays a role in DNA damage repair. USP1 interacts with UAF1 (USP1-associated factor 1) to form a complex that is required for the deubiquitinase activity. The USP1/UAF1 complex deubiquitinates mono-ubiquitinated PCNA (proliferating cell nuclear antigen) and mono-ubiquitinated FANCD2 (Fanconi anemia group complementation group D2), which are proteins that play important functions in translesion synthesis (TLS) and the Fanconi anemia (FA) pathway, respectively. The USP1/UAF1 complex also deubiquitinates Fanconi anemia complementation group I (FANCI). These two pathways are essential for repair of DNA damage induced by DNA cross-linking agents, such as cisplatin and mitomycin C (MMC).

The Poly (ADP-ribose) polymerase (PARP) family of enzymes plays roles in DNA repair and genome integrity. PARP is critical for single stranded break repair and base excision repair pathways. A key enzymatic activity is to add ADP-ribose to substrate protein via cleavage of NAD+ and release of nicotinamide. This poly (ADP-ribosyl)ation (“PARylation”) activity is activated by DNA strand breaks, which leads to addition of Par to PARP itself and other DNA repair enzymes. PARP is critical for the recruitment of DNA repair proteins to the damage sites.

Homologous recombination is a DNA repair process crucial for the accurate repair of DNA damage. BRCA1/2 genes, along with other Fanconi anemia pathway genes (e.g., RAD51D, NBN, ATM), are components of homologous recombination-mediated DNA repair. Mutations in the genes encoding homologous recombination factors play roles in the development of certain cancers. PARP inhibitors prevent the repair of DNA single-stranded breaks and promote the conversion of single-stranded breaks to double-stranded breaks, which creates synthetic lethality in cancer cells that lack proficient double-stranded break mechanisms such as homologous recombination.

There remains an unmet medical need for more effective therapies, e.g., combination therapies, for the treatment of cancers.

### BRIEF SUMMARY OF THE DISCLOSURE

Combinations of (i) a ubiquitin-specific-processing protease 1 (USP1) inhibitor or a pharmaceutically acceptable salt, hydrate, solvate, amorphous solid, or polymorph thereof, and (ii) a poly ADP-ribose polymerase (PARP) inhibitor, or a pharmaceutically acceptable salt, hydrate, solvate, amorphous solid, or polymorph thereof, are provided herein. Also provided herein are methods of treating a subject with cancer using such a combination.

In one aspect, the present disclosure relates to a method of treating cancer in a subject who previously received treatment with a first poly ADP-ribose polymerase (PARP) inhibitor, the method comprising administering to the subject a ubiquitin-specific-processing protease (USP1) inhibitor and a second PARP inhibitor, wherein the first and the second PARP inhibitors are the same or different PARP inhibitors.

In one aspect, the subject did not previously receive treatment with a USP1 inhibitor.

In one aspect, the treatment with the first PARP inhibitor was interrupted or discontinued. In one aspect, the interruption is for at least one week, at least two weeks, at least three weeks, or at least four weeks. In one aspect, the interruption is for no more than four weeks.

In one aspect, the subject experienced unacceptable toxicity and/or unacceptable adverse reactions during treatment with the first PARP inhibitor.

In one aspect, the unacceptable toxicity or adverse reaction was a hematological toxicity such as thrombocytopenia, anemia, or neutropenia, pneumonitis, dyspnea, fever, cough, wheezing, a radiological abnormality, hypertension, myelodysplastic syndrome/acute myeloid leukemia (MDS/AML), nausea, and/or fatigue.

In one aspect, during treatment with the first PARP inhibitor, the dose of the first PARP inhibitor was reduced. In one aspect, the dose of the first PARP inhibitor was reduced to one quarter, one third, one half, two thirds, or three quarters of the dose prior to the reduction.

In one aspect, the first PARP inhibitor was olaparib and the dose prior to the reduction was 400 mg taken twice daily. In one aspect, the first PARP inhibitor was olaparib, and the dose after the reduction was 200 mg taken twice daily or 100 mg taken twice daily.

In one aspect, the first PARP inhibitor was niraparib and the dose prior to reduction was 300 mg taken once daily. In one aspect, the first PARP inhibitor was niraparib, and the dose after the reduction was 200 mg taken once daily or 100 mg taken once daily.

In one aspect, the first PARP inhibitor was talazoparib and the dose prior to reduction was 1 mg taken once daily. In one aspect, the first PARP inhibitor was talazoparib, and the dose after the reduction was 0.75 mg taken once daily, 0.5 mg taken once daily, or 0.25 mg taken once daily.

In one aspect, the first PARP inhibitor was rucaparib and the dose prior to reduction was 600 mg taken twice daily. In one aspect, the first PARP inhibitor was rucaparib, and the dose after the reduction was 500 mg taken twice daily, 400 mg taken twice daily, or 300 mg taken twice daily.

In one aspect, the first PARP inhibitor was olaparib, niraparib, talazoparib, or rucaparib. In one aspect, the second PARP inhibitor is olaparib, niraparib, talazoparib, or rucaparib. In one aspect, the first PARP inhibitor was olaparib and the second PARP inhibitor is olaparib. In one aspect, the first PARP inhibitor was niraparib and the second PARP inhibitor is niraparib. In one aspect, the first PARP inhibitor was talazoparib and the second PARP inhibitor is talazoparib. In one aspect, the first PARP inhibitor was rucaparib and the second PARP inhibitor is rucaparib.

In one aspect, the first PARP inhibitor and the second PARP inhibitor are the same PARP inhibitor. In one aspect, the first PARP inhibitor and the second PARP inhibitor are different PARP inhibitors.

In one aspect, the dose of the second PARP inhibitor is reduced compared to the dose of first PARP inhibitor.

In one aspect, the USP1 inhibitor is a compound selected from the group consisting of

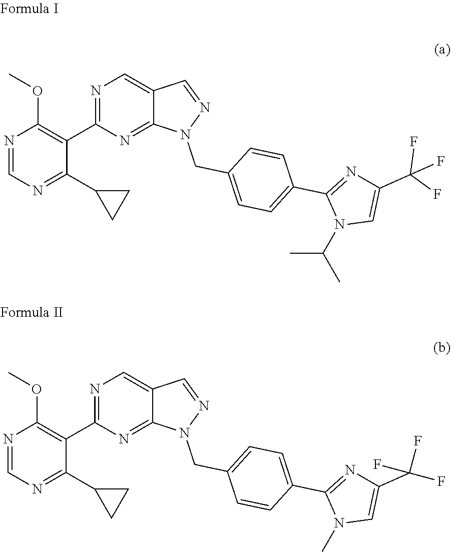 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1]

and pharmaceutically acceptable salts, hydrates, solvates, amorphous solids, or polymorphs thereof.

In one aspect, the USP1 inhibitor is a compound selected from the group consisting of

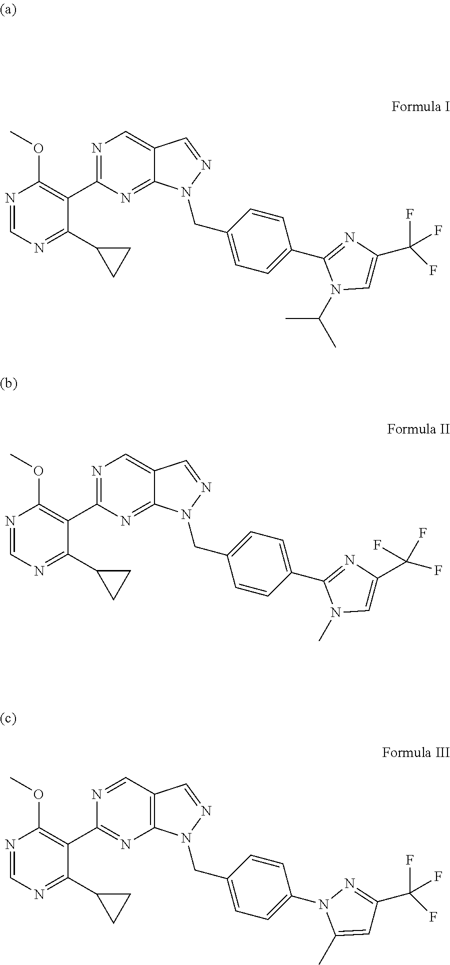 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1 ; COc1ncnc(C2CC2)c1-c1ncc2cnn(Cc3ccc(-n4nc(C(F)(F)F)cc4C)cc3)c2n1]

and pharmaceutically acceptable salts, hydrates, solvates, amorphous solids, or polymorphs thereof.

In one aspect, the USP1 inhibitor and the second PARP inhibitor are well tolerated.

In one aspect, the USP1 inhibitor decreases the exposure of the subject to the second PARP inhibitor.

In one aspect, the USP1 inhibitor and the second PARP inhibitor inhibit rebounding and/or regrowth of the cancer.

In one aspect, the USP1 inhibitor and the second PARP inhibitor are administered sequentially. In one aspect, the USP1 inhibitor and the second PARP inhibitor are administered simultaneously.

In one aspect, the subject is human.

In one aspect, the cancer is selected from the group consisting of brain cancer, lung cancer, non-small cell lung cancer (NSCLC), colon cancer, bladder cancer, osteosarcoma, ovarian cancer, skin cancer, uterine cancer, peritoneal cancer, and endometrial cancer, and breast cancer. In one aspect, the cancer is breast cancer. In one aspect, the breast cancer is triple negative breast cancer (TNBC). In one aspect, the cancer is a BRCA1 mutant cancer, a BRCA2 mutant cancer, or a BRCA1 mutant and BRCA2 mutant cancer. In one aspect, the cancer is ovarian cancer. In one aspect, the ovarian cancer is a BRCA1 mutant cancer, a BRCA2 mutant cancer, or a p53 mutant cancer. In one aspect, the ovarian cancer is a BRCA1 mutant cancer and a p53 mutant cancer. In one aspect, the ovarian cancer is a BRCA1 and BRCA2 mutant cancer. In one aspect, the ovarian cancer is a BRCA2 mutant cancer. In one aspect, the cancer is selected from the group consisting of a hematological cancer and a lymphatic cancer.

In one aspect, the cancer comprises cells with elevated levels of RAD51. In one aspect, the elevated levels of RAD51 are elevated RAD51 protein levels. In one aspect, the elevated levels of RAD51 are elevated RAD51 protein foci levels. In one aspect, at least 10% of cells that are in the S/G2 phase of the cell cycle in a sample obtained from the cancer are RAD51-positive. In one aspect, the elevated levels of RAD51 are elevated RAD51 mRNA levels. In one aspect, the elevated levels of RAD51 have been detected prior to the administration. In one aspect, the method further comprises detecting RAD51 levels in a cancer sample obtained from the subject prior to the administration.

In one aspect, the cancer is selected from the group consisting of a DNA damage repair pathway deficient cancer, a homologous-recombination deficient cancer, a cancer comprising cancer cells with a mutation in a gene encoding p53, a cancer comprising cancer cells with a loss of function mutation in a gene encoding p53, and a cancer comprising cells with a mutation in the gene encoding ATM.

In one aspect, the cancer is a PARP inhibitor resistant or refractory cancer.

In one aspect, the present disclosure relates to a method of treating cancer in a subject comprising administering to the subject a USP1 inhibitor, wherein the cancer comprises cancer cells with elevated levels of RAD51. In one aspect, the elevated levels of RAD51 have been detected prior to the administration. In one aspect, the method further comprises detecting RAD51 levels in a cancer sample obtained from the subject. In one aspect, the method further comprises administering to the subject a PARP inhibitor in combination with the USP1 inhibitor. In one aspect, the PARP inhibitor is olaparib, niraparib, talazoparib, or rucaparib.

In one aspect, the present disclosure relates to a method of selecting a subject with cancer for treatment with a USP1 inhibitor, comprising detecting whether the cancer comprises cells with elevated levels of RAD51, wherein if the cancer comprises cells with elevated levels of RAD51, the subject is selected for treatment with a USP1 inhibitor.

In one aspect, the present disclosure relates to an in vitro method for identifying a subject with cancer to be responsive to the treatment with a USP1 inhibitor, comprising detecting RAD51 levels in a cancer sample obtained from the subject, wherein elevated levels of RAD51 in the cancer sample are indicative for the patient to be responsive to the treatment with a USP1 inhibitor.

In one aspect, the present disclosure relates to an in vitro use of at least one agent capable of specifically detecting RAD51, for identifying a subject with cancer to be responsive to the treatment with a USP1 inhibitor.

In one aspect of any method or use provided herein, the treatment with a USP1 inhibitor further comprises treatment with a PARP inhibitor in combination with the USP1 inhibitor. In one aspect, the PARP inhibitor is olaparib, niraparib, talazoparib, or rucaparib. In one aspect, the USP1 inhibitor is a compound selected from the group consisting of

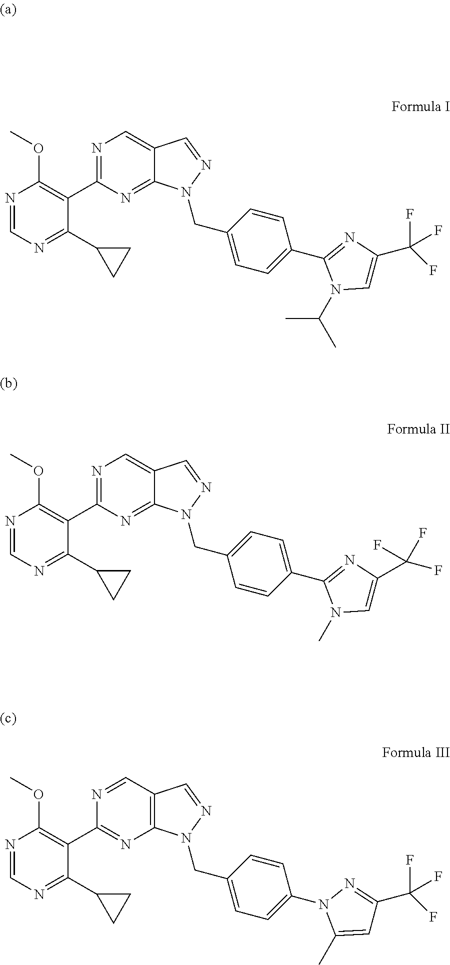 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1 ; COc1ncnc(C2CC2)c1-c1ncc2cnn(Cc3ccc(-n4nc(C(F)(F)F)cc4C)cc3)c2n1]

and pharmaceutically acceptable salts, hydrates, solvates, amorphous solids, or polymorphs thereof.

In one aspect of any method or use provided herein, the subject is human. In one aspect, the cancer is selected from the group consisting of brain cancer, lung cancer, non-small cell lung cancer (NSCLC), colon cancer, bladder cancer, osteosarcoma, ovarian cancer, skin cancer, uterine cancer, peritoneal cancer, and endometrial cancer, and breast cancer. In one aspect, the cancer is breast cancer. In one aspect, the breast cancer is triple negative breast cancer (TNBC). In one aspect, the cancer is a BRCA1 mutant cancer, a BRCA2 mutant cancer, or a BRCA1 mutant and BRCA2 mutant cancer. In one aspect, the cancer is ovarian cancer. In one aspect, the ovarian cancer is a BRCA1 mutant cancer, a BRCA2 mutant cancer, or a p53 mutant cancer. In one aspect, the ovarian cancer is a BRCA1 mutant cancer and a p53 mutant cancer. In one aspect, the ovarian cancer is a BRCA1 and BRCA2 mutant cancer. In one aspect, the ovarian cancer is a BRCA2 mutant cancer.

In one aspect, the present disclosure relates to a method of delaying, reducing, or preventing rebounding of a tumor in a subject comprising administering to the subject (i) a ubiquitin-specific-processing protease 1 (USP1) inhibitor and (ii) a poly ADP-ribose polymerase (PARP) inhibitor, or a pharmaceutically acceptable salt, hydrate, solvate, amorphous solid, or polymorph thereof, wherein the USP1 inhibitor is a compound selected from the group consisting of

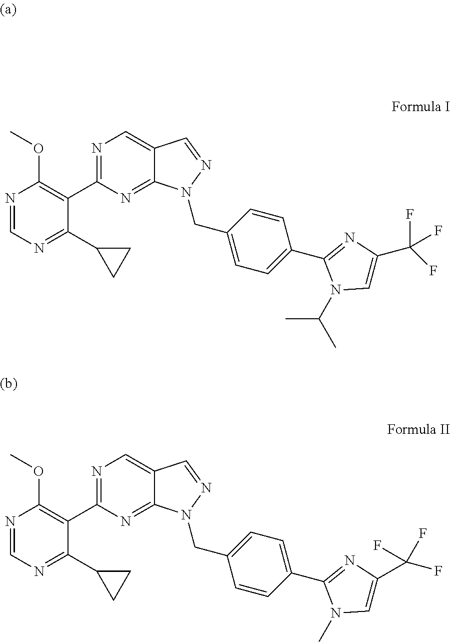 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1]

and pharmaceutically acceptable salts, hydrates, solvates, amorphous solids, or polymorphs thereof.

In one aspect, the present disclosure relates to a method of delaying, reducing, or preventing rebounding of a tumor in a subject comprising administering to the subject (i) a ubiquitin-specific-processing protease 1 (USP1) inhibitor and (ii) a poly ADP-ribose polymerase (PARP) inhibitor, or a pharmaceutically acceptable salt, hydrate, solvate, amorphous solid, or polymorph thereof, wherein the USP1 inhibitor is a compound selected from the group consisting of

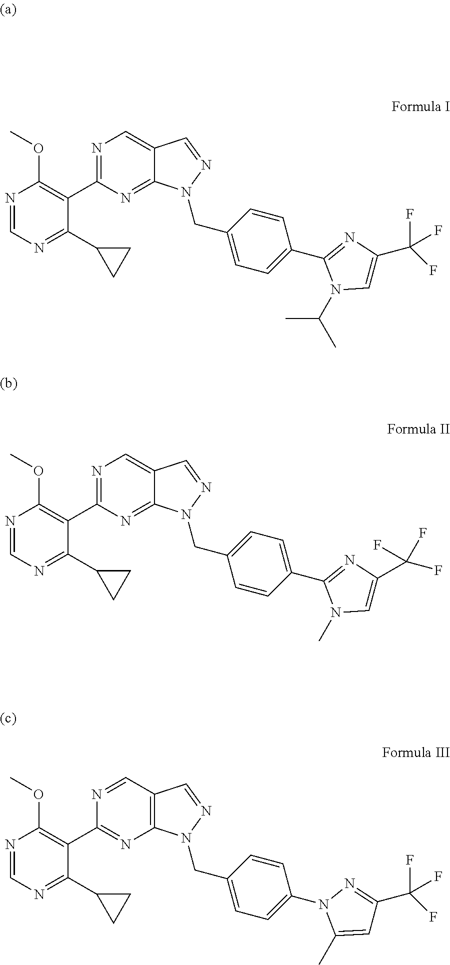 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1 ; COc1ncnc(C2CC2)c1-c1ncc2cnn(Cc3ccc(-n4nc(C(F)(F)F)cc4C)cc3)c2n1]

and pharmaceutically acceptable salts, hydrates, solvates, amorphous solids, or polymorphs thereof

In one aspect, the present disclosure relates to a combination composition comprising (i) a ubiquitin-specific-processing protease 1 (USP1) inhibitor and (ii) a poly ADP-ribose polymerase (PARP) inhibitor, or a pharmaceutically acceptable salt, hydrate, solvate, amorphous solid, or polymorph thereof, wherein the USP1 inhibitor is a compound selected from the group consisting of

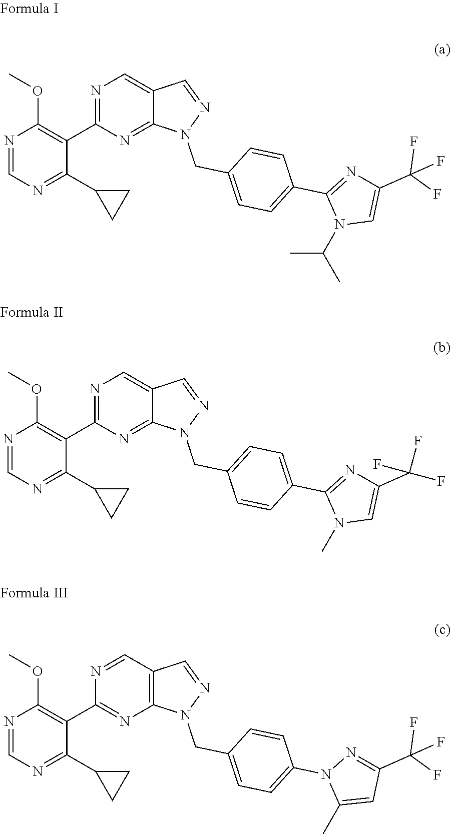 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1 ; COc1ncnc(C2CC2)c1-c1ncc2cnn(Cc3ccc(-n4nc(C(F)(F)F)cc4C)cc3)c2n1]

and pharmaceutically acceptable salts, hydrates, solvates, amorphous solids, or polymorphs thereof.

In one aspect, the present disclosure relates to a combination composition comprising (i) a ubiquitin-specific-processing protease 1 (USP1) inhibitor and (ii) a poly ADP-ribose polymerase (PARP) inhibitor, or a pharmaceutically acceptable salt, hydrate, solvate, amorphous solid, or polymorph thereof, wherein the USP1 inhibitor is a compound selected from the group consisting of

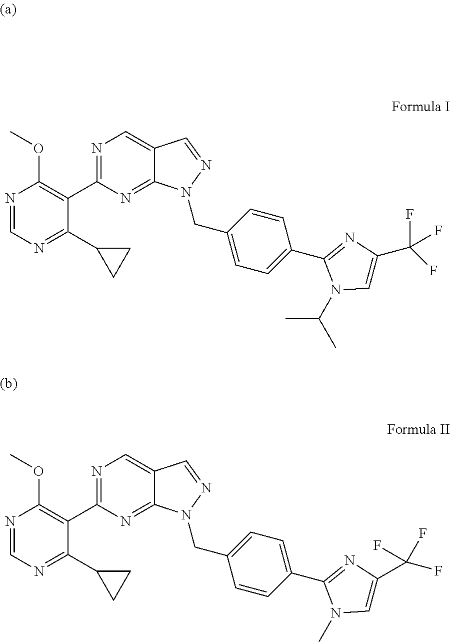 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1]

and pharmaceutically acceptable salts, hydrates, solvates, amorphous solids, or polymorphs thereof.

In one aspect, the present disclosure relates to a combination composition comprising (i) a ubiquitin-specific-processing protease 1 (USP1) inhibitor and (ii) a poly ADP-ribose polymerase (PARP) inhibitor, or a pharmaceutically acceptable salt, hydrate, solvate, amorphous solid, or polymorph thereof, wherein the USP1 inhibitor is a compound selected from the group consisting of

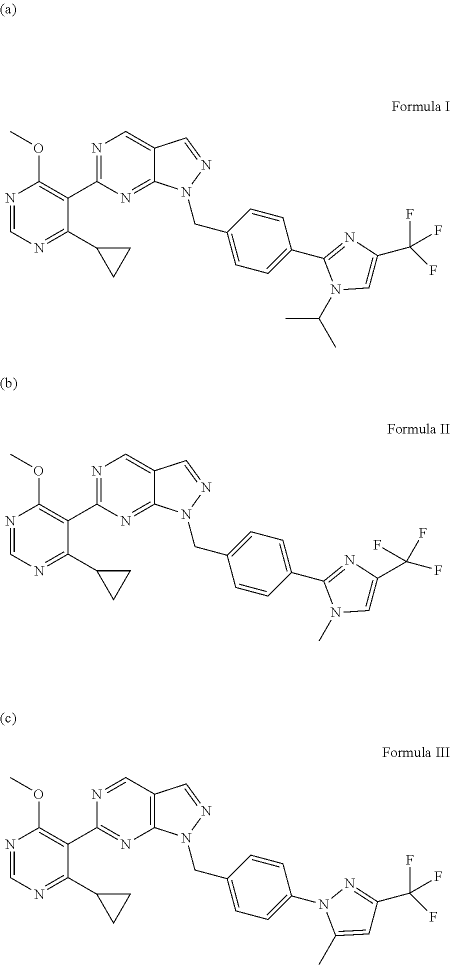 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1 ; COc1ncnc(C2CC2)c1-c1ncc2cnn(Cc3ccc(-n4nc(C(F)(F)F)cc4C)cc3)c2n1]

and pharmaceutically acceptable salts, hydrates, solvates, amorphous solids, or polymorphs thereof.

In some aspects, the PARP inhibitor is selected from the group consisting of olaparib (Lynparza®), rucaparib (Rubraca®), niraparib (Zejula®), and talazoparib (Talzenna®), and pharmaceutically acceptable salts, hydrates, solvates, amorphous solids, or polymorphs thereof.

In one aspect, the PARP inhibitor is niraparib or a pharmaceutically acceptable salt, hydrate, solvate, amorphous solid, or polymorph thereof.

In another aspect, the PARP inhibitor is olaparib or a pharmaceutically acceptable salt, hydrate, solvate, amorphous solid, or polymorph thereof.

In one aspect, the USP1 inhibitor is the compound of Formula I, or a pharmaceutically acceptable salt, hydrate, solvate, amorphous solid, or polymorph thereof.

In another aspect, the USP1 inhibitor is the compound of Formula II, or a pharmaceutically acceptable salt, hydrate, solvate, amorphous solid, or polymorph thereof.

In another aspect, the USP1 inhibitor is the compound of Formula III, or a pharmaceutically acceptable salt, hydrate, solvate, amorphous solid, or polymorph thereof.

In one aspect, the present disclosure relates to the use of the combination composition for the manufacture of a medicament for treatment of cancer.

In another aspect, the present disclosure relates to a pharmaceutical combination composition comprising the combination composition and a pharmaceutically acceptable carrier.

In one aspect, the pharmaceutical composition is for use in the treatment of cancer.

In one aspect, the present disclosure relates to a kit comprising the combination composition or the pharmaceutical combination composition, and instructions for administering the combination to a subject having cancer.

In another aspect, the present disclosure relates to a method of treating cancer in a subject comprising administering to the subject (i) USP1 inhibitor and (ii) PARP inhibitor, or a pharmaceutically acceptable salt, hydrate, solvate, amorphous solid, or polymorph thereof,

wherein the USP1 inhibitor is a compound selected from the group consisting of

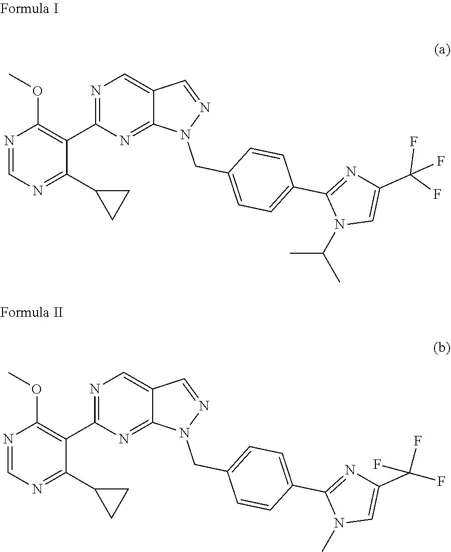 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1]

and pharmaceutically acceptable salts, hydrates, solvates, amorphous solids, or polymorphs thereof.

In another aspect, the present disclosure relates to a method of treating cancer in a subject comprising administering to the subject (i) USP1 inhibitor and (ii) PARP inhibitor, or a pharmaceutically acceptable salt, hydrate, solvate, amorphous solid, or polymorph thereof,

wherein the USP1 inhibitor is a compound selected from the group consisting of

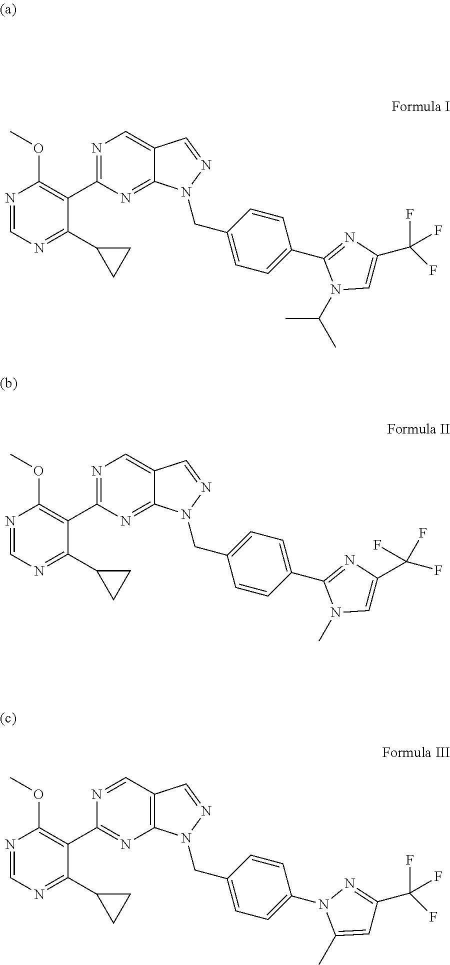 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1 ; COc1ncnc(C2CC2)c1-c1ncc2cnn(Cc3ccc(-n4nc(C(F)(F)F)cc4C)cc3)c2n1]

and pharmaceutically acceptable salts, hydrates, solvates, amorphous solids, or polymorphs thereof.

In some aspects of the method, the PARP inhibitor is selected from the group consisting of niraparib, olaparib and pharmaceutically acceptable salts, hydrates, solvates, amorphous solids, or polymorphs thereof.

In one aspect of the method, the PARP inhibitor is niraparib or a pharmaceutically acceptable salt, hydrate, solvate, amorphous solid, or polymorph thereof.

In another aspect of the method, the PARP inhibitor is olaparib or a pharmaceutically acceptable salt, hydrate, solvate, amorphous solid, or polymorph thereof.

In one aspect of the method, the USP1 inhibitor is the compound of Formula I, or a pharmaceutically acceptable salt, hydrate, solvate, amorphous solid, or polymorph thereof.

In another aspect of the method, the USP1 inhibitor is the compound of Formula II, or a pharmaceutically acceptable salt, hydrate, solvate, amorphous solid, or polymorph thereof.

In another aspect of the method, the USP1 inhibitor is the compound of Formula III, or a pharmaceutically acceptable salt, hydrate, solvate, amorphous solid, or polymorph thereof.

In one aspect of the present disclosure, the administration of the USP1 inhibitor, or said pharmaceutically acceptable salt, hydrate, solvate, amorphous solid, or polymorph thereof, and the PARP inhibitor, or said pharmaceutically acceptable salt, hydrate, solvate, amorphous solid, or polymorph thereof, provides a synergistic effect.

In one aspect of the present disclosure, the USP1 inhibitor, or said pharmaceutically acceptable salt, hydrate, solvate, amorphous solid, or polymorph thereof, and the PARP inhibitor, or said pharmaceutically acceptable salt, hydrate, solvate, amorphous solid, or polymorph thereof, are administered in a therapeutically effective amount sufficient to produce one or more therapeutic effects selected from the group consisting of (i) a reduction in tumor size, (ii) an increase in cancer tumor regression rate, (iii) a reduction or inhibition of cancer tumor growth, and (iv) a reduction of the toxicity effects of a PARP inhibitor administered as a monotherapy. In one aspect of the present disclosure, the USP1 inhibitor, or said pharmaceutically acceptable salt, hydrate, solvate, amorphous solid, or polymorph thereof, and the PARP inhibitor, or said pharmaceutically acceptable salt, hydrate, solvate, amorphous solid, or polymorph thereof, are administered in a therapeutically effective amount sufficient to produce one or more therapeutic effects selected from the group consisting of (i) a reduction in tumor size, (ii) an increase in cancer tumor regression rate, and (iii) a reduction or inhibition of cancer tumor growth. In one aspect of the present disclosure, the USP1 inhibitor, or said pharmaceutically acceptable salt, hydrate, solvate, amorphous solid, or polymorph thereof, and the PARP inhibitor, or said pharmaceutically acceptable salt, hydrate, solvate, amorphous solid, or polymorph thereof, are administered in an amount sufficient to reduce the toxicity effects of a PARP inhibitor administered as a monotherapy.

In one aspect, the USP1 inhibitor, or said pharmaceutically acceptable salt, hydrate, solvate, amorphous solid, or polymorph thereof, and the PARP inhibitor, or said pharmaceutically acceptable salt, hydrate, solvate, amorphous solid, or polymorph thereof, are administered in a therapeutically effective amount sufficient to delay, reduce, or prevent rebounding (rapid re-growth) of a tumor.

In one aspect, the USP1 inhibitor, or said pharmaceutically acceptable salt, hydrate, solvate, amorphous solid, or polymorph thereof, and the PARP inhibitor, or said pharmaceutically acceptable salt, hydrate, solvate, amorphous solid, or polymorph thereof, are administered sequentially.

In another aspect, the USP1 inhibitor, or said pharmaceutically acceptable salt, hydrate, solvate, amorphous solid, or polymorph thereof, and the PARP inhibitor, or said pharmaceutically acceptable salt, hydrate, solvate, amorphous solid, or polymorph thereof, are administered simultaneously.

In one aspect of the present disclosure, the combination is administered to a mammal. In another aspect, the mammal is a human.

In some aspects, the cancer is selected from the group consisting of a hematological cancer, a lymphatic cancer, a solid tumor, a DNA damage repair pathway deficient cancer and a homologous-recombination deficient cancer.

In some aspects, the cancer is selected from the group consisting of brain cancer, lung cancer, non-small cell lung cancer (NSCLC), colon cancer, bladder cancer, osteosarcoma, ovarian cancer, skin cancer, uterine cancer, peritoneal cancer, and endometrial cancer, and breast cancer.

In some aspects, the cancer is non-small cell lung cancer (NSCLC).

In some aspects, the cancer is colon cancer.

In some aspects, the cancer is bladder cancer.

In some aspects, the cancer is ovarian cancer or breast cancer.

In some aspects, the cancer is ovarian cancer.

In some aspects, the cancer is breast cancer.

In some aspects, the cancer is triple negative breast cancer.

In some aspects, the cancer is selected from the group consisting of bone cancer, including osteosarcoma and chondrosarcoma; brain cancer, including glioma, glioblastoma, astrocytoma, medulloblastoma, and meningioma; soft tissue cancer, including rhabdoid and sarcoma; kidney cancer; bladder cancer; skin cancer, including melanoma; and lung cancer, including non-small cell lung cancer; colon cancer, uterine cancer; nervous system cancer; head and neck cancer; pancreatic cancer; and cervical cancer.

In some aspects, the cancer is a DNA damage repair pathway deficient cancer.

In some aspects, the cancer is a BRCA1 mutant cancer. In some aspects, the BRCA1 mutation is a germline mutation. In some aspects, the BRCA1 mutation is a somatic mutation. In some aspects, the BRCA1 mutation leads to BRCA1 deficiency.

In some aspects, the cancer is a BRCA2 mutant cancer. In some aspects, the BRCA2 mutation is a germline mutation. In some aspects, the BRCA2 mutation is a somatic mutation. In some aspects, the BRCA2 mutation leads to BRCA2 deficiency.

In some aspects, the cancer is a BRCA1 mutant cancer and a BRCA2 mutant cancer.

In some aspects, the cancer is a BRCA1 deficient cancer.

In some aspects, the cancer is a BRCA2 deficient cancer.

In some aspects, the cancer is a BRCA1 deficient cancer and a BRCA2 deficient cancer.

In some aspects, the cancer is a PARP inhibitor refractory or resistant cancer. In some aspects, the cancer is a PARP inhibitor resistant or refractory BRCA1, BRCA2, or BRCA1 and BRCA2 mutant cancer. In some aspects, the cancer is a PARP inhibitor resistant or refractory BRCA1, BRCA2, or BRCA1 and BRCA2-deficient cancer.

In some aspects, the cancer has a mutation in the gene encoding ataxia telangiectasia mutated (ATM) protein kinase. In some aspects, the ATM mutation is a germline mutation. In some aspects, the ATM mutation is a somatic mutation. In some aspects, the cancer is an ATM-deficient cancer.

In some aspects, the cancer comprises cancer cells with a mutation in a gene encoding p53. In some aspects, the mutation in a gene encoding p53 is a germline mutation. In some aspects, the mutation in a gene encoding p53 is a somatic mutation. In some aspects, the cancer comprises cancer cells with a loss of function mutation in a gene encoding p53.

In some aspects, the cancer has a mutation in the gene encoding at least two of p53, BRCA1, BRCA2, and ATM.

In some aspects, the cancer comprises cells with elevated levels of RAD51. In some aspects, the elevated levels of RAD51 are elevated RAD51 protein levels. In some aspects, the elevated levels of RAD51 are elevated RAD51 protein foci levels. In some aspects, at least 10% of cells that are in the S/G2 phase of the cell cycle in a sample obtained from the cancer are RAD51-positive. In some aspects, the elevated levels of RAD51 are elevated RAD51 mRNA levels. In some aspects, the elevated levels of RAD51 have been detected prior to the administration or the treatment. In some aspects, a method or use provided herein further comprises detecting RAD51 levels in a cancer sample obtained from the subject prior to the administration or the treatment.

In another aspect, the present disclosure relates to a method of treating a USP1 protein mediated disorder and/or a PARP protein mediated disorder comprising administering to a subject in need thereof a USP1 inhibitor of Formula I or II, or a pharmaceutically acceptable salt, hydrate, solvate, amorphous solid, or polymorph thereof, and a PARP inhibitor, or a pharmaceutically acceptable salt, hydrate, solvate, amorphous solid, or polymorph thereof, in an effective amount to treat the USP1 protein mediated disorder and/or the PARP protein mediated disorder. In another aspect, the present disclosure relates to a method of treating a USP1 protein mediated disorder and/or a PARP protein mediated disorder comprising administering to a subject in need thereof a USP1 inhibitor of Formula I, Formula II, or Formula III, or a pharmaceutically acceptable salt, hydrate, solvate, amorphous solid, or polymorph thereof, and a PARP inhibitor, or a pharmaceutically acceptable salt, hydrate, solvate, amorphous solid, or polymorph thereof, in an effective amount to treat the USP1 protein mediated disorder and/or the PARP protein mediated disorder.

In another aspect, the present disclosure relates to a method of inhibiting a USP1 protein and/or a PARP protein comprising contacting a USP1 protein and/or a PARP protein with a USP1 inhibitor of Formula I or II, or a pharmaceutically acceptable salt, hydrate, solvate, amorphous solid, or polymorph thereof, and a PARP inhibitor, or a pharmaceutically acceptable salt, hydrate, solvate, amorphous solid, or polymorph thereof. In another aspect, the present disclosure relates to a method of inhibiting a USP1 protein and/or a PARP protein comprising contacting a USP1 protein and/or a PARP protein with a USP1 inhibitor of Formula I, Formula II, or Formula III, or a pharmaceutically acceptable salt, hydrate, solvate, amorphous solid, or polymorph thereof, and a PARP inhibitor, or a pharmaceutically acceptable salt, hydrate, solvate, amorphous solid, or polymorph thereof.

In some aspects, the contacting occurs in vitro.

In some aspects, the contacting occurs in vivo.

Additional aspects and advantages of the disclosure will be set forth, in part, in the description that follows, and will flow from the description, or can be learned by practice of the disclosure. The aspects and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### BRIEF DESCRIPTION OF THE FIGURES

FIG. 1 shows the synergistic effect of combining a USP1 inhibitor of Formula II and Niraparib in a JHOS2 BRCA1 mutant ovarian cancer model.

FIG. 2 shows the synergistic effect of combining a USP1 inhibitor of Formula II and Niraparib in a COV362 BRCA1 mutant ovarian cancer model.

FIG. 3 shows the synergistic effect of combining a USP1 inhibitor of Formula II and Niraparib in a UWB1.289 BRCA1 mutant ovarian cancer model.

FIGS. 4A and 4B show the anti-tumor activity of the USP1 inhibitor of Formula I free base in comparison to Olaparib and Niraparib in mice using the MDA-MB-436 BRCA1 mutant human breast tumor model.

FIGS. 5A and 5B show the anti-tumor activity of the USP1 inhibitor of Formula I co-crystal in comparison to Olaparib and Niraparib in mice using the MDA-MB-436 BRCA1 mutant human breast tumor model.

FIGS. 6A, 6B and 6C show the anti-tumor activity of the USP1 inhibitor of Formula I co-crystal in combination with the PARP inhibitor Olaparib in the MDA-MB-436 human breast tumor mouse xenograft model. FIGS. 6D and 6E show the enhanced anti-tumor activity of the USP1 inhibitor of Formula I co-crystal in combination with the PARP inhibitor Olaparib at day 27 (last measurement before dose termination; FIG. 6D) and at day 55 (27-days post-dose termination; FIG. 6E) in the MDA-MB-436 BRCA1 mutant human breast tumor mouse model. FIG. 6F shows that the combination of the USP1 inhibitor of Formula and the PARP inhibitor Olaparib is well-tolerated in the MDA-MB-436 BRCA1 mutant human breast tumor model.

FIGS. 7A, 7B, 7C, 7D and 7E show the anti-tumor activity of the USP1 inhibitor of Formula I co-crystal in combination with the PARP inhibitor Olaparib in patient-derived breast xenograft models in nude mice.

FIGS. 8A, 8B, 8C and 8D show the anti-tumor activity of the USP1 inhibitor of Formula I co-crystal in combination with the PARP inhibitor Olaparib in the HBCx-11 BRCA1 mutant HRD high human breast PDX model. FIG. 8A shows the activity of the combination as compared to the monotherapies. FIGS. 8B and 8C show the activity of Olaparib monotherapy at 50 mg/kg (FIG. 8B) and at 100 mg/kg (FIG. 8C) in individual mice. FIG. 8D shows the activity of the combination in individual mice. FIG. 8E shows that the combination of the USP1 inhibitor of Formula I co-crystal and the PARP inhibitor Olaparib is well-tolerated in the HBCx-11 BRCA1 mutant HRD high human breast PDX model

FIGS. 9A, 9B and 9C show the anti-tumor activity of the USP1 inhibitor of Formula I co-crystal in combination with the PARP inhibitor Olaparib in the HBCx-14 HRD high human breast PDX model. FIG. 9A shows the activity of the combination as compared to Olaparib monotherapy. FIG. 9B shows the activity of Olaparib monotherapy at 50 mg/kg in individual mice. FIG. 9C shows the activity of the combination in individual mice. FIG. 9D shows that the combination of the USP1 inhibitor of Formula I co-crystal and the PARP inhibitor Olaparib is well tolerated in the HBCx-14 HRD high human breast PDX model.

FIGS. 10A, 10B, 10C, 10D, 10E, 10F and 10G show the anti-tumor activity of the USP1 inhibitor of Formula I co-crystal in combination with the PARP inhibitor Olaparib in the OV0589 ovarian PDX BRCA1 and TP53 mutant model. FIG. 10A shows the activity of the combination as compared to the monotherapies. FIG. 10B shows activity of vehicle control in individual mice. FIGS. 10C and 10D show the activity of the USP1 inhibitor of Formula I co-crystal at 100 mg/kg (FIG. 10C) and 300 mg/kg (FIG. 10D) in individual mice. FIGS. 10E and 10F show the activity of Olaparib monotherapy at 50 mg/kg (FIG. 10E) and at 100 mg/kg (FIG. 10F in individual mice. FIG. 10G shows the activity of the combination in individual mice. FIG. 10G shows the activity of the combination in individual mice. FIG. 10H shows that the combination of the USP1 inhibitor of Formula I co-crystal and the PARP inhibitor Olaparib is well tolerated in the OV0589 ovarian PDX BRCA1 and TP53 mutant model.

FIG. 11A shows that none of the USP1 inhibitor of Formula I, the PARP inhibitor Olaparib, or the combination thereof have activity in the ST416 ovarian BRCA1 mutant PDX model. FIG. 11B shows the tolerability of the USP1 inhibitor of Formula I, the PARP inhibitor Olaparib, and the combination thereof in the ST416 ovarian BRCA1 mutant PDX model.

FIGS. 12A, 12B, 12C and 12D show that the USP1 inhibitor of Formula I enhances the activity of the PARP inhibitor Niraparib in the MDA-MB-436 TNBC CDX BRCA1 mutant human breast tumor model. FIG. 12A shows the activity of the combination as compared to the monotherapies. FIGS. 12B and 12C show the activity of Niraparib monotherapy at 20 mg/kg (FIG. 12B) and at 50 mg/kg (FIG. 12C) in individual mice. FIG. 12D shows the activity of the combination in individual mice. FIG. 12E shows that the combination of the USP1 inhibitor of and the PARP inhibitor Niraparib is well-tolerated in the MDA-MB-436 TNBC CDX BRCA1 mutant human breast tumor model.

FIGS. 13A, 13B, 13C and 13D show drug-drug interaction pharmacokinetics of Olaparib (FIGS. 13A and 13B) and Formula I (FIGS. 13C and 13D) in combination in NOD SCID mice at Day 1 (FIGS. 13A and 13C) and Day 5 (FIGS. 13B and 13D).

FIG. 14 shows the synergistic effect of combining a USP1 inhibitor of Formula I and Olaparib in HCT116 ovarian cancer cells.

FIGS. 15A, 15B, 15C, 15D, 15E and 15F show that none of the USP1 inhibitor of Formula I, the PARP inhibitor Olaparib, or the combination thereof have activity in the CTG-0253 ovarian PDX model. FIG. 15A shows the activity of the combination as compared to the monotherapies. FIG. 15B shows the activity of vehicle control in individual mice. FIG. 15C shows the activity of the USP1 inhibitor of Formula I co-crystal at 100 mg/kg in individual mice. FIG. 15D shows the activity of the USP1 inhibitor of Formula I co-crystal at 300 mg/kg in individual mice. FIG. 15E shows the activity of Olaparib monotherapy at 100 mg/kg in individual mice. FIG. 15F shows the activity of the combination (100 mg/kg Olaparib+100 mg/kg Formula I) in individual mice. FIG. 15G shows the tolerability of the USP1 inhibitor of Formula I, the PARP inhibitor Olaparib, and the combination thereof in the CTG-0253 ovarian PDX model.

FIGS. 16A, 16B, 16C, 16D and 16E show the anti-tumor activity of the USP1 inhibitor of Formula I co-crystal in combination with the PARP inhibitor Olaparib in the HBCx-8 PDX BRCA1 and TP53 mutant Olaparib-resistant model. FIG. 16A shows the activity of the combination as compared to the monotherapies. FIG. 16B shows activity of vehicle control in individual mice. FIG. 16C shows the activity of the USP1 inhibitor of Formula I co-crystal at 100 mg/kg in individual mice. FIG. 16D shows the activity of Olaparib monotherapy at 100 mg/kg in individual mice. FIG. 16E shows the activity of the combination in individual mice. FIG. 16F shows that the combination of the USP1 inhibitor of Formula I co-crystal and the PARP inhibitor Olaparib is well tolerated in the HBCx8 TNBC ovarian PDX BRCA1 and TP53 mutant Olaparib-resistant model.

FIGS. 17A, 17B, 17C, 17D, 17E, 17F and 17G show the anti-tumor activity and tolerability of the USP1 inhibitor of Formula I co-crystal in combination with the PARP inhibitor Olaparib in the HBCx-17 breast PDX BRCA2 and TP53 mutant, HRD high model. FIG. 17A shows the activity of the combination as compared to the monotherapies. FIG. 17B shows that the combination of the USP1 inhibitor of Formula I co-crystal and the PARP inhibitor Olaparib is well tolerated in the HBCx-17 model. FIG. 17C shows the anti-tumor activity of the USP1 inhibitor of Formula I co-crystal in combination with the PARP inhibitor Olaparib (50 mg/kg) in the HBCx-17 model. FIG. 17D shows activity of vehicle control in individual mice. FIG. 17E show the activity of the USP1 inhibitor of Formula I co-crystal at 100 mg/kg in individual mice. FIG. 17F shows the activity of Olaparib monotherapy at 50 mg/kg in individual mice. FIG. 17G shows the activity of the combination (Olaparib 50 mg/kg and Formula I 100 mg/kg) in individual mice. FIG. 17H shows the anti-tumor activity of combination of the USP1 inhibitor of Formula I co-crystal and the PARP inhibitor Olaparib (100 mg/kg) in the HBCx-17 model. FIG. 17I shows activity of vehicle control in individual mice. FIG. 17J show the activity of the USP1 inhibitor of Formula I co-crystal at 100 mg/kg in individual mice. FIG. 17K shows the activity of Olaparib monotherapy at 100 mg/kg in individual mice. FIG. 17L shows the activity of the combination (Olaparib 100 mg/kg and Formula I 100 mg/kg) in individual mice.

FIGS. 18A, 18B, 18C, 18D, 18E, 18F, 18G, 18H, 18I, 18J, 18K and 18L show the anti-tumor activity and tolerability of the USP1 inhibitor of Formula I co-crystal in combination with the PARP inhibitor Olaparib in the CTG-0703 BRCA1 and TP53 mutant ovarian PDX model. FIG. 18A shows the activity of the combination as compared to the monotherapies. FIG. 18B shows that the combination of the USP1 inhibitor of Formula I co-crystal and the PARP inhibitor Olaparib is well tolerated in the CTG-0703 model. FIG. 18C shows the anti-tumor activity of Formula I co-crystal in combination with the PARP inhibitor Olaparib (50 mg/kg) in the CTG-0703 model. FIG. 18D shows activity of vehicle control in individual mice. FIG. 18E shows the activity of the USP1 inhibitor of Formula I co-crystal at 100 mg/kg in individual mice. FIG. 18F shows the activity of Olaparib monotherapy at 50 mg/kg in individual mice. FIG. 18G shows the activity of the combination in individual mice. FIG. 18H shows the anti-tumor activity of Formula I co-crystal in combination with the PARP inhibitor Olaparib (100 mg/kg) in the CTG-0703 model. FIG. 18I shows activity of vehicle control in individual mice. FIG. 18J shows the activity of the USP1 inhibitor of Formula I co-crystal at 100 mg/kg in individual mice.

FIG. 18K shows the activity of Olaparib monotherapy at 100 mg/kg in individual mice.

FIG. 18L shows the activity of the combination in individual mice.

FIG. 19 shows that in CRISPR-Cas9 resistance screens, the representation of positive control guides decreased, whereas the representation of neutral control guides did not, at Day 4 (D4), Day 7 (D7), and Day 14 (D14).

FIG. 20 shows a volcano plot with genes that have differential viability with Formula I co-crystal treatment. The data represents the enrichment of MDA-MB-436 cells at Day 14 (D14) vs. Day 0 (DO) after treatment with the USP1 inhibitor of Formula I co-crystal (USPi) and knockout of various genes (e.g., RAD18 and UBE2A).

### DETAILED DESCRIPTION OF THE DISCLOSURE

One aspect of the present disclosure is based on the use of a combination of a ubiquitin-specific-processing protease 1 (USP1) protein inhibitor and a poly ADP-ribose polymerase (PARP) inhibitor. The combinations are useful for inhibiting a USP1 protein and/or a PARP protein and for treating diseases, disorders, or conditions, e.g., cancer, that are responsive to inhibition of a USP1 protein and/or a PARP protein.

In some aspects, the combination of a USP1 inhibitor and a PARP inhibitor provide a synergistic effect.

In some aspects, the USP1 inhibitor and the PARP inhibitor are in therapeutically effective amounts sufficient to produce a therapeutic effect comprising: (i) a reduction in size of a tumor, (ii) an increase in cancer tumor regression rate, (iii) a reduction or inhibition of cancer tumor growth, and/or (iv) a reduction of the toxicity effects of a PARP inhibitor administered as a monotherapy. In some aspects, the USP1 inhibitor and the PARP inhibitor can delay, reduce, or prevent rebounding (rapid re-growth) of a tumor.

The tolerability (lack of toxicity) of combinations provided herein is particular surprising given that other combinations with the PARP inhibitor Olaparib have not been well-tolerated. See, e.g., Samol, J., et al., Invest. New Drugs, 30:1493-500 (2012) (“Further development of olaparib and topotecan in combination was not explored due to dose-limiting hematological AEs and the resulting sub-therapeutic MTD.”).

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

It is understood that embodiments described herein include “consisting” and/or “consisting essentially of” embodiments. As used herein, the singular form “a,” “an,” and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about,” as used herein, includes the recited number±10%. Thus, “about 10” means 9 to 11. As is understood by one skilled in the art, reference to “about” a value or parameter herein includes (and describes) instances that are directed to that value or parameter per se. For example, description referring to “about X” includes description of “X.”

The present disclosure encompasses the preparation and use of salts of the USP1 inhibitors and PARP inhibitors, including non-toxic pharmaceutically acceptable salts. Examples of pharmaceutically acceptable addition salts include inorganic and organic acid addition salts and basic salts. Pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like. The term “pharmaceutically acceptable salt” as used herein, refers to any salt, e.g., obtained by reaction with an acid or a base, of a USP1 inhibitor or PARP inhibitor of the disclosure that is physiologically tolerated in the target patient (e.g., a mammal, e.g., a human).

Acid addition salts can be formed by mixing a solution of the particular USP1 inhibitor or PARP inhibitor with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, or the like. Basic salts can be formed by mixing a solution of the USP1 inhibitor or PARP inhibitor of the present disclosure with a solution of a pharmaceutically acceptable non-toxic base such as sodium hydroxide, potassium hydroxide, choline hydroxide, sodium carbonate and the like.

In some aspects of the disclosure, a pharmaceutically acceptable salt is formed between a compound of Formula I or Formula II and a pharmaceutically acceptable acid. In some aspects of the disclosure, a pharmaceutically acceptable salt is formed between a compound of Formula I, Formula II, or Formula III and a pharmaceutically acceptable acid. In some aspects, the pharmaceutically acceptable acid is selected from the group consisting of 1-hydroxy-2-naphthoic acid, 4-aminosalicylic acid, ascorbic acid, adipic acid, L-aspartic acid, benzene sulfonic acid, benzoic acid, trans-cinnamic acid, citric acid, ethanedisulfonic acid, fumaric acid, galactaric acid, gallic acid, gentisic acid, gluconic acid, D-glucuronic acid, glutamic acid, glutaric acid, glycolic acid, hexanoic acid, hippuric acid, hydrobromic acid, hydrochloric acid, lactic acid, maleic acid, L-malic acid, malonic acid, R-mandelic acid, methanesulfonic acid, mucic acid, naphthalene sulfonic acid, nicotinic acid, oxalic acid, palmitic acid, p-toluene sulfonic acid, phosphoric acid, propionic acid, saccharin, salicylic acid, stearic acid, succinic acid, sulfuric acid, L-tartaric acid, vanillic acid, and vanillin. In some aspects, the pharmaceutically acceptable acid is selected from the group consisting of benzoic acid, gallic acid, gentisic acid and salicylic acid.

The present disclosure encompasses the preparation and use of solvates of the USP1 inhibitor and/or PARP inhibitor. Solvates typically do not significantly alter the physiological activity or toxicity of the compounds, and as such may function as pharmacological equivalents. The term “solvate” as used herein is a combination, physical association and/or solvation of a USP1 inhibitor or PARP inhibitor of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. USP1 inhibitors or PARP inhibitors of the disclosure can be present as solvated forms with a pharmaceutically acceptable solvent, such as water, methanol, ethanol, and the like, and it is intended that the disclosure includes both solvated and unsolvated forms of the USP1 inhibitor and/or PARP inhibitor of the disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E.C. van Tonder et al., AAPS Pharm. Sci. Tech., 5(1):Article 12 (2004), and A. L. Bingham et al., Chem. Commun. 603-604 (2001). Atypical, non-limiting, process of preparing a solvate would involve dissolving a USP1 inhibitor or PARP inhibitor of the disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

In some aspects of the disclosure, the USP1 inhibitor and/or PARP inhibitor is deuterated. In some aspects, the USP1 inhibitor and/or PARP inhibitor are partially or completely deuterated, i.e., one or more hydrogen atoms are replaced with deuterium atoms.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

In the context of cancer, the term “treating” includes, but is not limited to, inhibiting growth of cancer cells, inhibiting replication of cancer cells, lessening of overall tumor burden, and delaying, halting, or slowing tumor growth, progression, or metastasis.

As used herein, “delaying” means to defer, hinder, slow, retard, stabilize, suppress and/or postpone development or progression of the disease (such as cancer). This delay can be of varying lengths of time, depending on the history of the disease and/or individual being treated.

A “therapeutically effective amount” of a substance can vary according to factors such as the disease state, age, sex, and weight of the individual, and the ability of the substance to elicit a desired response in the individual. A therapeutically effective amount is also one in which any toxic or detrimental effects of the substance are outweighed by the therapeutically beneficial effects. A therapeutically effective amount can be delivered in one or more administrations. A therapeutically effective amount refers to an amount effective, at dosages and for periods of time necessary, to achieve the desired therapeutic effect.

The terms “combination,” “therapeutic combination,” “combination composition,” “combination therapy” or “pharmaceutical combination”, as used herein, can include a fixed combination in one dosage unit form, separate dosage units or a kit of parts or instructions for the combined administration where the USP1 inhibitor and the PARP inhibitor can be administered independently at the same time or separately within time intervals. A combined pharmaceutical composition can be adapted for simultaneous, separate, or sequential administration.

The combination therapy can provide “synergy” and prove “synergistic,” i.e., the effect achieved when the active ingredients used together is greater than the sum of the effects that results from using the compounds separately. A synergistic effect can include a significantly reduced effective dose for the combination of the two active ingredients as compared to the effective dose of each active ingredient when administered separately. A synergistic effect can also include a reduction in toxicity for the combination of the two active ingredients as compared to the toxicity of each active ingredient when administered separately. A synergistic effect can also be an effect that cannot be achieved by administration of any of the active ingredients as single agents. The synergistic effect can include, but is not limited to, an effect of treating cancer by reducing tumor size, inhibiting tumor growth, or increasing survival of the subject. The synergistic effect can also include reducing cancer cell viability, inducing cancer cell death, and inhibiting or delaying cancer cell growth. A synergistic effect can be attained, for example, when the active ingredients are: (1) co-formulated and administered or delivered simultaneously in a combined, unit dosage formulation; (2) delivered serially, by alternation, or in parallel as separate formulations; or (3) by some other regimen. When delivered in alternation therapy, a synergistic effect can be attained when the compounds are administered or delivered sequentially.

A determination of a synergistic interaction between a USP1 inhibitor and a PARP inhibitor can be based on the results obtained from the assays described herein. For example, combination effects can be evaluated using the Bliss independence model. Bliss scores quantify degree of potentiation from single agents, and a Bliss score >0 suggests greater than simple additivity. In some aspects, a Bliss score greater than 10 indicates strong synergy. In other aspects, a score of 6 or greater indicates synergy. In some aspects, the Bliss score is about 6, about 7, about 8, about 9, about 10, about 11, about 12, about 13, about 14, about 15, about 16, about 17, about 18, about 19, about 20 or about 25.

As used herein, a “homologous recombination deficiency score” or “HRD score” means an algorithmic assessment of three measures of tumor genomic instability, i.e., loss of heterozygosity, telomeric allelic imbalance and large-scale state transitions.

The terms “administer,” “administering,” “administration,” and the like refer to methods that can be used to enable delivery of the therapeutic agent to the desired site of biological action. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa. Administration of two or more therapeutic agents includes simultaneous (concurrent) and consecutive administration in any order.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

The term “container” means any receptacle and closure therefore suitable for storing, shipping, dispensing, and/or handling a pharmaceutical product.

The term “insert” or “package insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

The term “disease” or “condition” or “disorder” as used herein refers to a condition where treatment is needed and/or desired and denotes disturbances and/or anomalies that as a rule are regarded as being pathological conditions or functions, and that can manifest themselves in the form of particular signs, symptoms, and/or malfunctions. As demonstrated below, combinations of the USP1 inhibitors and PARP inhibitors of the present disclosure can be used in treating diseases and conditions, such as proliferative diseases, wherein inhibition of USP1 and/or PARP proteins provides a benefit.

The terms “polypeptide” and “protein” are used interchangeably to refer to a polymer of amino acid residues and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“USP1” and “ubiquitin-specific-processing protease 1” as used herein refer to any native polypeptide or USP1-encoding polynucleotide. The term “USP1” encompasses “full-length,” unprocessed USP1 polypeptide as well as any forms of USP1 that result from processing within the cell (e.g., removal of the signal peptide). The term also encompasses naturally occurring variants of USP1, e.g., those encoded by splice variants and allelic variants. The USP1 polypeptides described herein can be isolated from a variety of sources, such as from human tissue types or from another source, or prepared by recombinant or synthetic methods. Human USP1 sequences are known and include, for example, the sequences publicly available as UniProt No. 094782 (including isoforms). As used herein, the term “human USP1 protein” refers to USP1 protein comprising the amino acid sequence as set forth in SE ID NO:1:

(SEQ ID NO:1)
MPGVIPSESNGLSRGSPSKKNRLSLKFFQKKETKRALDFTDSQENEEK
ASEYRASEIDQVVPAAQSSPINCEKRENLLPFVGLNNLGNTCYLNSIL
QVLYFCPGFKSGVKHLFNIISRKKEALKDEANQKDKGNCKEDSLASYE
LICSLQSLIISVEQLQASFLLNPEKYTDELATQPRRLLNTLRELNPMY
EGYLQHDAQEVLQCILGNIQETCQLLKKEEVKNVAELPTKVEEIPHPK
EEMNGINSIEMDSMRHSEDFKEKLPKGNGKRKSDTEFGNMKKKVKLSE
HQSLEENQRQTRSKRKATSDTLESPPKIIPKYISENESPRPSQKKKSR
VKINWLKSATKQPSILSKFCSLGKITTNQGVKGQSKENECDPEEDLGK
CESDNTTNGCGLESPGNTVTPVNVNEVKPINKGEEQIGFELVEKLFQG
QLVLRTRCLECESLTERREDFQDISVPVQEDELSKVEESSEISPEPKT
EMKTLRWAISQFASVERIVGEDKYFCENCHHYTEAERSLLFDKMPEVI
TIHLKCFAASGLEFDCYGGGLSKINTPLLTPLKLSLEEWSTKPTNDSY
GLFAVVMHSGITISSGHYTASVKVTDLNSLELDKGNFVVDQMCEIGKP
EPLNEEEARGVVENYNDEEVSIRVGGNTQPSKVLNKKNVEAIGLLGGQ
KSKADYELYNKASNPDKVASTAFAENRNSETSDTTGTHESDRNKESSD
QTGINISGFENKISYVVQSLKEYEGKWLLFDDSEVKVTEEKDFLNSLS
PSTSPTSTPYLLFYKKL.

USP1 is a deubiquitinating enzyme that acts as part of a complex with UAF1. USP1's “deubiquitinase activity” includes its ability to deubiquitinate as part of the USP1-UAF1 complex.

“PARP” or “PARP protein” as used herein refers to one or more of the Poly (ADP-ribose) polymerase family of enzymes. The family includes enzymes that have the ability to catalyze the transfer of ADP-ribose to target proteins (poly ADP-ribosylation). There are at least 18 members of the PARP family that are encoded by different genes, and share homology in a conserved catalytic domain, including PARP-1, PARP-2 and PARP-3.

The term “specifically binds” is well understood in the art, and methods to determine such specific binding are also well known in the art. A molecule is said to exhibit “specific binding” or “preferential binding” if it reacts or associates more frequently, more rapidly, with greater duration and/or with greater affinity with a particular protein or domain of a protein than it does with alternative proteins or domains. It should be understood that a molecule that specifically or preferentially binds to a first protein or domain may or may not specifically or preferentially bind to a second protein or domain. As such, “specific binding” or “preferential binding” does not necessarily require (although it can include) exclusive binding. Generally, but not necessarily, reference to binding means preferential binding. For example, a USP1 inhibitor that specifically binds to USP1, UAF1, and/or the USP1-UAF1 complex may not bind to other deubiquitinases, other USP proteins, or other UAF1 complexes (e.g., USP46-UAF1) or may bind to other deubiquitinases, other USP proteins, or other UAF1 complexes (e.g., USP46-UAF1) with a reduced affinity as compared to binding to USP1.

The terms “reduction” or “reduce” or “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control over the same period of time.

In some aspects, inhibiting USP1 proteins is the inhibition of one or more activities or functions of USP1 proteins. It should be appreciated that the activity or function of the one or more USP1 proteins may be inhibited in vitro or in vivo. Non-limiting examples of activities and functions of USP1 include deubiquitinase activity and formation of a complex with UAF1 and are described herein. Exemplary levels of inhibition of the activity of one or more USP1 proteins include at least 10% inhibition, at least 20% inhibition, at least 30% inhibition, at least 40% inhibition, at least 50% inhibition, at least 60% inhibition, at least 70% inhibition, at least 80% inhibition, at least 90% inhibition, and up to 100% inhibition.

In some aspects, inhibiting PARP proteins is the inhibition of one or more activities or functions of PARP proteins. It should be appreciated that the activity or function of the one or more PARP proteins may be inhibited in vitro or in vivo. Non-limiting examples of activities and functions of PARP are described herein. Exemplary levels of inhibition of the activity of one or more PARP proteins include at least 10% inhibition, at least 20% inhibition, at least 30% inhibition, at least 40% inhibition, at least 50% inhibition, at least 60% inhibition, at least 70% inhibition, at least 80% inhibition, at least 90% inhibition, and up to 100% inhibition.

The terms “individual” or “subject” are used interchangeably herein to refer to an animal, for example, a mammal, such as a human. In some instances, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some instances, the subject to receive the treatment can be a patient, designating the fact that the subject has been identified as having a disorder of relevance to the treatment, or being at particular risk of contracting the disorder.

As used herein, the terms “cancer” and “tumor” refer to or describe the physiological condition in mammals in which a population of cells are characterized by unregulated cell growth. The terms encompass solid and hematological/lymphatic cancers. Examples of cancer include but are not limited to, DNA damage repair pathway deficient cancers. Additional examples of cancer include, but are not limited to, ovarian cancer, breast cancer (including triple negative breast cancer), non-small cell lung cancer (NSCLC), and osteosarcoma. The cancer can be BRCA1 or BRCA2 wild type. The cancer can also be BRCA1 or BRCA2 mutant. The cancer can further be a PARP inhibitor resistant or refractory cancer, or a PARP inhibitor resistant or refractory BRCA1 or BRCA2-mutant cancer.

As used herein, the term “loss of function” mutation refers to a mutation that results in the absence of a gene, decreased expression of a gene, or the production of a gene product (e.g. protein) having decreased activity or no activity. Loss of function mutations include for example, missense mutations, nucleotide insertions, nucleotide deletions, and gene deletions. Loss of function mutations also include dominant negative mutations. Thus, cancer cells with a loss of function mutation in a gene encoding p53 include cancer cells that contain missense mutations in a gene encoding p53 as well as cancer cells that lack a gene encoding p53.

### USP1 Inhibitors

In some aspects, the ubiquitin-specific-processing protease 1 (USP1) inhibitor of the disclosure comprises a compound of

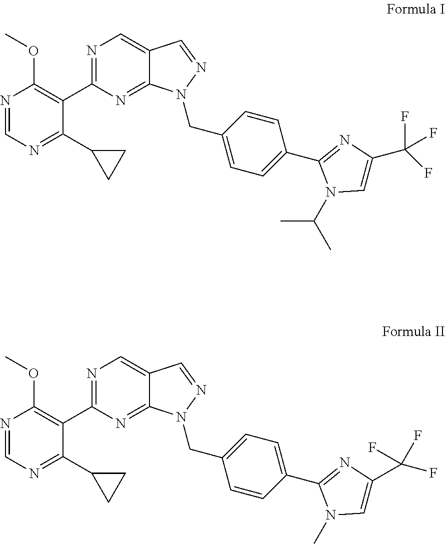 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1]

or a pharmaceutically acceptable salt, hydrate, solvate, amorphous solid, or polymorph thereof.

The chemical name for the USP1 inhibitor of Formula I is 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine, as described in U.S. application Ser. No. 16/721,079.

The chemical name for the USP1 inhibitor of Formula II is 6-(4-cyclopropyl-6-methoxypyrimidin-5-yl)-1-(4-(1-methyl-4-(trifluoromethyl)-1H-imidazol-2-yl)benzyl)-1H-pyrazolo[3,4-d]pyrimidine, as described in U.S. application Ser. No. 16/721,079.

U.S. application Ser. No. 16/721,079 is herein incorporated by reference in its entirely.

In some aspects, the ubiquitin-specific-processing protease 1 (USP1) inhibitor of the disclosure comprises a compound of

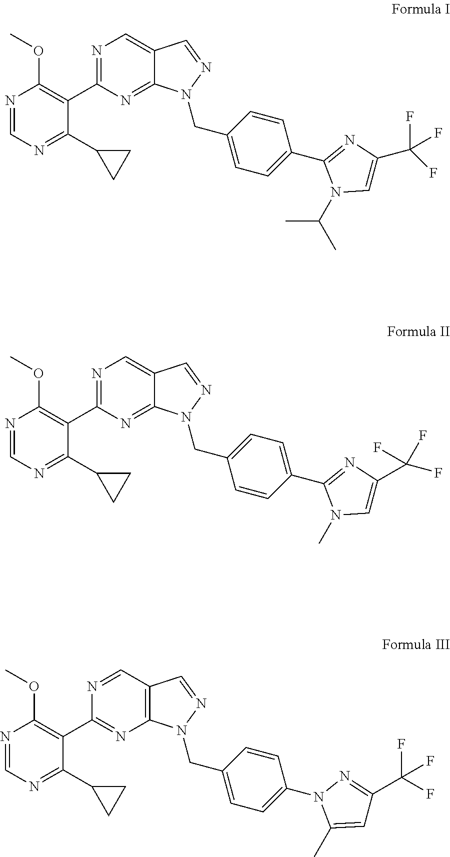 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1 ; COc1ncnc(C2CC2)c1-c1ncc2cnn(Cc3ccc(-n4nc(C(F)(F)F)cc4C)cc3)c2n1]

or a pharmaceutically acceptable salt, hydrate, solvate, amorphous solid, or polymorph thereof.

The chemical name for the USP1 inhibitor of Formula III is 6-(4-cyclopropyl-6-methoxypyrimidin-5-yl)-1-(4-(5-methyl-3-(trifluoromethyl)-1H-pyrazol-1-yl)benzyl)-1H-pyrazolo[3,4-d]pyrimidine, as described in U.S. application Ser. No. 16/721,079. U.S. application Ser. No. 16/721,079 is herein incorporated by reference in its entirely.

In various aspects, the USP1 inhibitors reduce the level of USP1 protein and/or inhibit or reduce at least one biological activity of USP1 protein.

In some aspects, the USP1 inhibitors specifically bind to USP1 protein. In some aspects, the USP1 inhibitors specifically bind to USP1 protein in a USP1-UAF1 complex. In some aspects, the USP1 inhibitors specifically bind to USP1 mRNA. In some aspects, the USP1 inhibitors specifically bind to USP1 protein (alone or in a USP1-UAF1 complex) or USP1 mRNA. In some aspects, the USP1 inhibitors specifically bind to UAF1 (alone or in a USP1-UAF1 complex) and inhibit or reduces formation or activity of the USP1-UAF1 complex.

In some aspects, the USP1 inhibitors decrease the formation of the USP1-UAF1 complex. In some aspects, the USP1 inhibitors decrease the activity of the USP1-UAF1 complex. In some aspects, the USP1 inhibitors decrease the deubiquitinase activity of USP1. In some aspects, the USP1 inhibitors increase mono-ubiquitinated PCNA. In some aspects, the USP1 inhibitors increase mono-ubiquitinated FANCD2. In some aspects, the USP1 inhibitors increase mono-ubiquitinated FANCI.

In some aspects, the USP1 inhibitors do not bind to other deubiquitinases, other USP proteins, or other UAF1 complexes (e.g., USP46-UAF1) or bind deubiquitinases, other USP proteins, or other UAF1 complexes (e.g., USP46-UAF1) with at least 5-fold, at least 10-fold, at least 20-fold, or at least 100-fold reduced affinity compared to the affinity for USP1 (i.e., the K_{D} of the USP1 inhibitor for other deubiquitinases, other USP proteins, or other UAF1 complexes (e.g., USP46-UAF1) is at least 5-fold, at least 10-fold, at least 20-fold, or at least 100-fold higher than the K_{D} for USP1).

In some aspects, the USP1 inhibitors inhibit USP1 deubiquitinase activity with an IC50 of less than about 50 nM, between about 50 nM and about 200 nM, between about 200 nM and about 2 pM, or greater than 2 pM, e.g., as measured using the assay disclosed in U.S. Patent Application Publication No. 2017/0145012 or IC50 of 50 nM to 1000 nM, e.g., as measured using the assay disclosed in Liang et al., Nat Chem Biol 10: 289-304 (2014). In some aspects, the USP1 inhibitors inhibit USP1 deubiquitinase activity with an IC50 as measured using the assay disclosed in Chen, et al., Chem Biol., 18(11):1390-1400 (2011). In some aspects, the USP1 inhibitors do not inhibit the activity of other deubiquitinases, other USP proteins, or other UAF1 complexes (e.g., USP46-UAF1) or inhibit the activity of other deubiquitinases, other USP proteins, or other UAF1 complexes (e.g., USP46-UAF1) with at least 5-fold, at least 10-fold, at least 20-fold, or at least 100-fold higher IC50 compared to the IC50 for inhibition of USP1 deubiquitinase activity.

In some aspects, the USP1 inhibitors of the present disclosure bind to a USP1 protein with an affinity in the range of 1 pM to 100 μM, or 1 pM to 1 μM, or 1 pM to 500 nM, or 1 pM to 100 nM. In some aspects, the USP1 inhibitors of the present disclosure bind to a USP1 protein with an affinity of about 1 pM to about 100 μM, about 1 nM to about 100 μM, about 1 μM to about 100 μM, about 1 μM to about 50 μM, about 1 μM to about 40 μM, about 1 μM to about 30 μM, about 1 μM to about 20 μM, or about 1 μM to about 10 μM, about 1 μM, about 5 μM, about 10 μM, about 15 μM, about 20 μM, about 25 μM, about 30 μM, about 35 μM, about 40 μM, about 45 μM, about 50 μM, about 60 μM, about 70 μM, about 80 μM, about 90 μM, or about 100 μM. In some aspects, the USP1 inhibitors of the present disclosure bind to a USP1 protein with an affinity of about 100 nM to about 1 μM, about 100 nM to about 900 nM, about 100 nM to about 800 nM, about 100 nM to about 700 nM, about 100 nM to about 600 nM, about 100 nM to about 500 nM, about 100 nM to about 400 nM, about 100 nM to about 300 nM, about 100 nM to about 200 nM, about 200 nM to about 1 μM, about 300 nM to about 1 μM, about 400 nM to about 1 μM, about 500 nM to about 1 μM, about 600 nM to about 1 μM, about 700 nM to about 1 μM, about 800 nM to about 1 μM, about 900 nM to about 1 μM, about 100 nM, about 200 nM, about 300 nM, about 400 nM, about 500 nM, about 600 nM, about 700 nM, about 800 nM, or about 900 nM. In some aspects, the USP1 inhibitors of the present disclosure bind to a USP1 protein with an affinity of about 1 nM to about 100 nM, 1 nM to about 90 nM, 1 nM to about 80 nM, 1 nM to about 70 nM, 1 nM to about 60 nM, 1 nM to about 50 nM, 1 nM to about 40 nM, 1 nM to about 30 nM, 1 nM to about 20 nM, 1 nM to about 10 nM, about 10 nM to about 100 nM, about 20 nM to about 100 nM, about 30 nM to about 100 nM, about 40 nM to about 100 nM, about 50 nM to about 100 nM, about 60 nM to about 100 nM, about 70 nM to about 100 nM, about 80 nM to about 100 nM, about 90 nM to about 100 nM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, or about 100 nM.

In some aspects, the USP1 inhibitors of the present disclosure bind to a USP1 protein with an affinity of less than 1 μM, less than 500 nM, less than 100 nM, less than 10 nM, or less than 1 nM. In some aspects, the USP1 inhibitors bind to a USP1 protein with an affinity of less than 1 nM.

In some aspects, the USP1 inhibitors of the present disclosure inhibit USP1 activity with an IC₅₀ of 1 pM to 100 μM, or 1 pM to 1 μM, or 1 pM to 500 nM, or 1 pM to 100 nM. In some aspects, the USP1 inhibitors inhibit USP1 activity with an IC₅₀ of about 1 pM to about 100 μM, about 1 nM to about 100 μM, about 1 μM to about 100 μM, about 1 μM to about 50 μM, about 1 μM to about 40 μM, about 1 μM to about 30 μM, about 1 μM to about 20 μM, or about 1 μM to about 10 μM, about 1 μM, about 5 μM, about 10 μM, about 15 μM, about 20 μM, about 25 μM, about 30 μM, about 35 μM, about 40 μM, about 45 μM, about 50 μM, about 60 μM, about 70 μM, about 80 μM, about 90 μM, or about 100 μM. In some aspects, the USP1 inhibitors inhibit USP1 activity with an IC₅₀ of about 100 nM to about 1 μM, about 100 nM to about 900 nM, about 100 nM to about 800 nM, about 100 nM to about 700 nM, about 100 nM to about 600 nM, about 100 nM to about 500 nM, about 100 nM to about 400 nM, about 100 nM to about 300 nM, about 100 nM to about 200 nM, about 200 nM to about 1 μM, about 300 nM to about 1 μM, about 400 nM to about 1 μM, about 500 nM to about 1 μM, about 600 nM to about 1 μM, about 700 nM to about 1 μM, about 800 nM to about 1 μM, about 900 nM to about 1 μM, about 100 nM, about 200 nM, about 300 nM, about 400 nM, about 500 nM, about 600 nM, about 700 nM, about 800 nM, or about 900 nM.

In some aspects, the USP1 inhibitors of the present disclosure inhibit USP1 activity with an IC₅₀ of about 1 nM to about 100 nM, 1 nM to about 90 nM, 1 nM to about 80 nM, 1 nM to about 70 nM, 1 nM to about 60 nM, 1 nM to about 50 nM, 1 nM to about 40 nM, 1 nM to about 30 nM, 1 nM to about 20 nM, 1 nM to about 10 nM, about 10 nM to about 100 nM, about 20 nM to about 100 nM, about 30 nM to about 100 nM, about 40 nM to about 100 nM, about 50 nM to about 100 nM, about 60 nM to about 100 nM, about 70 nM to about 100 nM, about 80 nM to about 100 nM, about 90 nM to about 100 nM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, or about 100 nM. In some aspects, the USP1 inhibitors inhibit USP1 activity with an IC₅₀ of less than 1 μM, less than 500 nM, less than 100 nM, less than 10 nM, or less than 1 nM. In some aspects, the USP1 inhibitors inhibit USP1 activity with an IC₅₀ of less than 1 nM.

Other exemplary USP1 inhibitors are disclosed, for example, in WO 2020/132269 and U.S. Provisional Application 62/857,986, each of which is herein incorporated by reference in its entirety.

### Exemplary Assays for Inhibition of USP1

Any suitable assay in the art can be used to determine an activity, detect an outcome or effect, or determine efficacy. See, e.g. U.S. application Ser. No. 16/721,079. U.S. application Ser. No. 16/721,079 is herein incorporated by reference in its entirely.

In some instances, a method of determining whether a USP1 inhibitor compound inhibits USP1 deubiquitinase activity measures a change in mass upon di-ubiquitin cleavage of deubiquitinase binding. For example, ubiquitin aldehyde and ubiquitin vinyl sulfone form covalent irreversible linkages to deubiquitinases that result in observable mass changes to the deubiquitinases. Similarly, cleavage of di-ubiquitins results in an observable mass change.

In some instances, a method of determining whether a USP1 inhibitor compound inhibits USP1 deubiquitinase activity involves an increase in luminescence or fluorescence upon cleavage, e.g., that can be monitored on a plate reader. Such assays can use ubiquitin linked to a flurophore through a linker linkage, such as ubiquitin-7-amino-4-methylcoumarin (Ub-AMC) or ubiquitin-Rhodamine110. Such assays can also use a di-ubiquitin containing an isopeptide linkage. Exemplary di-ubiquitins can comprise a flurophore on one ubiquitin and a quencher on the other ubiquitin such that fluorescence increases with then di-ubiquitin is cleaved. Such assays can also use enzyme-coupled systems wherein ubiquitin is coupled to an enzyme that is only active in producing a fluorescence enzyme product when released from the ubiquitin.

### PARP Inhibitors

In various aspects, the PARP inhibitors of the disclosure reduce the level of one or more PARP proteins and/or inhibit or reduce at least one biological activity of one or more PARP proteins.

PARP inhibitors include, for example, olaparib (Lynparza®), rucaparib (Rubraca®), niraparib (Zejula®), and talazoparib (Talzenna®).

In one aspect, the PARP inhibitor is niraparib (Zejula®), which is sold as niraparib tosylate monohydrate. The chemical name for niraparib tosylate monohydrate is 2-{4-[(3S)-piperidin-3-yl]phenyl}-2Hindazole 7-carboxamide 4-methylbenzenesulfonate hydrate (1:1:1). The molecular formula of niraparib tosylate is C₂₆H₃₀N₄O₅S, and it has a molecular weight of 492.6 g/mol.

Niraparib is an inhibitor of poly(ADP-ribose) polymerase (PARP) enzymes, PARP-1 and PARP-2, which play a role in DNA repair. In vitro studies have shown that niraparib-induced cytotoxicity may involve inhibition of PARP enzymatic activity and increased formation of PARP-DNA complexes resulting in DNA damage, apoptosis and cell death. Increased niraparib-induced cytotoxicity was observed in tumor cell lines with or without deficiencies in BRCA1/2. Niraparib decreased tumor growth in mouse xenograft models of human cancer cell lines with deficiencies in BRCA1/2 and in human patient-derived xenograft tumor models with homologous recombination deficiency that had either mutated or wild type BRCA1/2.

In another aspect, the PARP inhibitor is olaparib (Lynparza®). The chemical name is 4-[(3-{[4-(cyclopropylcarbonyl)piperazin-1-yl]carbonyl}-4-fluorophenyl)-methyl]phthalazin-1(2H)-one. The molecular formula is C₂₄H₂₃FN₄O₃, and the molecular weight is 434.5 g/mol.

Olaparib is an inhibitor of poly (ADP-ribose) polymerase (PARP) enzymes, including PARP1, PARP2, and PARP3. Olaparib has been shown to inhibit growth of select tumor cell lines in vitro and decrease tumor growth in mouse xenograft models of human cancer, both as monotherapy or following platinum-based chemotherapy. Increased cytotoxicity and anti-tumor activity following treatment with olaparib were noted in cell lines and mouse tumor models with deficiencies in BRCA and non-BRCA proteins involved in the homologous recombination repair (HRR) of DNA damage and correlated with platinum response. In vitro studies have shown that olaparib-induced cytotoxicity may involve inhibition of PARP enzymatic activity and increased formation of PARP-DNA complexes, resulting in DNA damage and cancer cell death.

In one aspect, the PARP inhibitors are used in anti-cancer combination therapies with USP1 inhibitors of the present disclosure. In addition to the PARP inhibitor and USP1 inhibitor, other therapies can be used either before, during or after the combination therapy.

### Exemplary Assays for Inhibition of PARP

The present disclosure provides compounds that are active in inhibiting the activity of PARP. Any suitable assay in the art can be used to determine an activity, detect an outcome or effect, or determine efficacy. See, e.g., Dillon, et al., JBS., 8(3), 347-352 (2003); U.S. Pat. No. 9,566,276.

In some aspects, the PARP inhibitors of the disclosure inhibit PARP activity with an IC₅₀ of less than about 50 nM, between about 50 nM and about 200 nM, between about 200 nM and about 2 pM, or greater than 2 pM.

In some aspects, the PARP inhibitors of the disclosure bind to a PARP protein with an affinity in the range of 1 pM to 100 μM, or 1 pM to 1 μM, or 1 pM to 500 nM, or 1 pM to 100 nM. In some aspects, the PARP inhibitors of the disclosure bind to a PARP protein with an affinity of about 1 pM to about 100 μM, about 1 nM to about 100 μM, about 1 μM to about 100 μM, about 1 μM to about 50 μM, about 1 μM to about 40 μM, about 1 μM to about 30 μM, about 1 μM to about 20 μM, or about 1 μM to about 10 μM, about 1 μM, about 5 μM, about 10 μM, about 15 μM, about 20 μM, about 25 μM, about 30 μM, about 35 μM, about 40 μM, about 45 μM, about 50 μM, about 60 μM, about 70 μM, about 80 μM, about 90 μM, or about 100 μM. In some aspects, the PARP inhibitors of the disclosure bind to a PARP protein with an affinity of about 100 nM to about 1 μM, about 100 nM to about 900 nM, about 100 nM to about 800 nM, about 100 nM to about 700 nM, about 100 nM to about 600 nM, about 100 nM to about 500 nM, about 100 nM to about 400 nM, about 100 nM to about 300 nM, about 100 nM to about 200 nM, about 200 nM to about 1 μM, about 300 nM to about 1 μM, about 400 nM to about 1 μM, about 500 nM to about 1 μM, about 600 nM to about 1 μM, about 700 nM to about 1 μM, about 800 nM to about 1 μM, about 900 nM to about 1 μM, about 100 nM, about 200 nM, about 300 nM, about 400 nM, about 500 nM, about 600 nM, about 700 nM, about 800 nM, or about 900 nM. In some aspects, the PARP inhibitors of the disclosure bind to a PARP protein with an affinity of about 1 nM to about 100 nM, 1 nM to about 90 nM, 1 nM to about 80 nM, 1 nM to about 70 nM, 1 nM to about 60 nM, 1 nM to about 50 nM, 1 nM to about 40 nM, 1 nM to about 30 nM, 1 nM to about 20 nM, 1 nM to about 10 nM, about 10 nM to about 100 nM, about 20 nM to about 100 nM, about 30 nM to about 100 nM, about 40 nM to about 100 nM, about 50 nM to about 100 nM, about 60 nM to about 100 nM, about 70 nM to about 100 nM, about 80 nM to about 100 nM, about 90 nM to about 100 nM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, or about 100 nM. In some aspects, the PARP inhibitors of the disclosure bind to a PARP protein with an affinity of less than 1 μM, less than 500 nM, less than 100 nM, less than 10 nM, or less than 1 nM. In some aspects, the PARP inhibitors of the disclosure bind to a PARP protein with an affinity of less than 1 nM.

In some aspects, the PARP inhibitors of the disclosure inhibit PARP activity with an IC₅₀ of 1 pM to 100 μM, or 1 pM to 1 μM, or 1 pM to 500 nM, or 1 pM to 100 nM. In some aspects, the PARP inhibitors of the disclosure inhibit PARP activity with an IC₅₀ of about 1 pM to about 100 μM, about 1 nM to about 100 μM, about 1 μM to about 100 μM, about 1 μM to about 50 μM, about 1 μM to about 40 μM, about 1 μM to about 30 μM, about 1 μM to about 20 μM, or about 1 μM to about 10 μM, about 1 μM, about 5 μM, about 10 μM, about 15 μM, about 20 μM, about 25 μM, about 30 μM, about 35 μM, about 40 μM, about 45 μM, about 50 μM, about 60 μM, about 70 μM, about 80 μM, about 90 μM, or about 100 μM. In some aspects, the PARP inhibitors of the disclosure inhibit PARP activity with an IC₅₀ of about 100 nM to about 1 μM, about 100 nM to about 900 nM, about 100 nM to about 800 nM, about 100 nM to about 700 nM, about 100 nM to about 600 nM, about 100 nM to about 500 nM, about 100 nM to about 400 nM, about 100 nM to about 300 nM, about 100 nM to about 200 nM, about 200 nM to about 1 μM, about 300 nM to about 1 μM, about 400 nM to about 1 μM, about 500 nM to about 1 μM, about 600 nM to about 1 μM, about 700 nM to about 1 μM, about 800 nM to about 1 μM, about 900 nM to about 1 μM, about 100 nM, about 200 nM, about 300 nM, about 400 nM, about 500 nM, about 600 nM, about 700 nM, about 800 nM, or about 900 nM. In some aspects, the PARP inhibitors of the disclosure inhibit PARP activity with an IC₅₀ of about 1 nM to about 100 nM, 1 nM to about 90 nM, 1 nM to about 80 nM, 1 nM to about 70 nM, 1 nM to about 60 nM, 1 nM to about 50 nM, 1 nM to about 40 nM, 1 nM to about 30 nM, 1 nM to about 20 nM, 1 nM to about 10 nM, about 10 nM to about 100 nM, about 20 nM to about 100 nM, about 30 nM to about 100 nM, about 40 nM to about 100 nM, about 50 nM to about 100 nM, about 60 nM to about 100 nM, about 70 nM to about 100 nM, about 80 nM to about 100 nM, about 90 nM to about 100 nM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, or about 100 nM. In some aspects, the PARP inhibitors of the disclosure inhibit PARP activity with an IC₅₀ of less than 1 μM, less than 500 nM, less than 100 nM, less than 10 nM, or less than 1 nM. In some aspects, the PARP inhibitors of the disclosure inhibit PARP activity with an IC₅₀ of less than 1 nM.

### Sensitive Cancers and Methods of Identifying Sensitive Cancers

As demonstrated herein, cancers comprising cells with elevated levels of RAD51 are sensitive to USP1 inhibitors and/or combinations of USP1 inhibitors and PARP inhibitors. The elevated levels of RAD51 can be elevated RAD51 protein levels, elevated RAD51 protein foci levels, and/or elevated RAD51 mRNA level.

Various methods of identifying a cancer as a USP1 inhibitor-sensitive cancer and/or a cancer that is sensitive to the combination of USP1 inhibitors and PARP inhibitors are provided herein. In some instances, such methods comprise detecting RAD51 (e.g., RAD51 protein, RAD51 protein foci, and/or RAD51 mRNA) levels in cancer cells (e.g., using a sample obtained from the cancer). RAD51 protein levels can be detected using, for example, immunofluorescence, western blots, fluorescence-activated cell sorting (FACS), and/or immunohistochemistry. RAD51 mRNA levels can be detected, for example, using quantitative reverse transcriptase (RT)-polymerase chain reaction (PCR). Elevated levels of RAD51 protein and/or mRNA indicate that a cancer is sensitive to USP1 inhibitors or to combinations of USP1 inhibitors and PARP inhibitors.

Methods of detecting RAD51 and RAD51 protein foci are provided, for example, in Castroviejo-Bermejo, Marta, et al., EMBO Molecular Medicine 10(12):e9172 (2018), which is herein incorporated by reference in its entirety. RAD51 can be detected, for example, using immunofluorescence. RAD51 foci, e.g., of 0.42-1.15 μm diameter can be quantified on formalin-fixed paraffin embedded (FFPE) tumor samples, by scoring the percentage of cells in the S/G2-cell cycle phase (e.g., geminin-positive cells) with 5 or more RAD51 nuclear foci. In some aspects, cancers comprising cells with elevated levels of RAD51 are cancers wherein at least 10% of cells that are in the S/G2 phase of the cell cycle (e.g., geminin-positive cells) are RAD51-positive.

In some aspects, a method of selecting a subject with cancer for treatment with a USP1 inhibitor (optionally in combination with a PARP inhibitor) comprises determining whether the cancer comprises cells with elevated levels of RAD51, wherein if the cancer comprises cells with elevated levels of RAD51, the subject is selected for treatment with a USP1 inhibitor, optionally in combination with a PARP inhibitor.

A cancer with elevated levels of RAD51 can be a homologous-recombination deficient cancer. A cancer with elevated levels of RAD51 can be a BRCA1 mutant cancer. A cancer with elevated levels of RAD51 can be a BRCA2 mutant cancer. A cancer with elevated levels of RAD51 can be a BRCA1 mutant and BRCA2 mutant cancer. A cancer with elevated levels of RAD51 can be cancer with deleterious or suspected deleterious mutations in BRCA1 and BRCA2 genes and/or a positive Genomic Instability Score, e.g., as determined using myChoice® CDx (Myriad®).

### Methods of Use

Since combinations of the disclosure are inhibitors of USP1 proteins and PARP proteins, the present disclosure provides a method for inhibiting a USP1 protein and/or a PARP protein comprising contacting a USP1 and/or PARP protein or a composition comprising a USP1 and/or PARP protein with one or more combinations of the disclosure.

Since combinations of the disclosure are inhibitors of USP1 and PARP proteins, a number of diseases, conditions, or disorders mediated by USP1 and/or PARP proteins can be treated by employing these compounds. The present disclosure is thus directed generally to a method for treating a disease, condition, or disorder responsive to the inhibition of USP1 and/or PARP proteins in an animal suffering from, or at risk of suffering from, the disorder, the method comprising administering to the animal an effective amount of one or more combinations of the disclosure.

The present disclosure is further directed to a method of inhibiting USP1 and/or PARP proteins in an animal in need thereof, the method comprising administering to the animal a therapeutically effective amount of a combination of the disclosure.

In some aspects, the combinations of the disclosure can be used to inhibit the activity of a USP1 and/or PARP protein. For example, in some aspects, a method of inhibiting a USP1 and/or PARP protein comprises contacting the USP1 and/or PARP protein with a combination of the disclosure. The contacting can occur in vitro or in vivo.

In some aspects, the combinations of the disclosure can be used to treat a USP1 and/or PARP protein mediated disorder. A USP1 and/or PARP protein mediated disorder is any pathological condition in which a USP1 and/or PARP protein is known to play a role. In some aspects, a USP1 and/or PARP mediated disorder is a proliferative disease such as cancer. In some aspects, the combinations of the disclosure can delay, reduce, or prevent rebounding (rapid re-growth) of a tumor. In some aspects, the combination of the disclosure is not significantly more toxic than the USP1 inhibitor alone. In some aspects, the combination of the disclosure is not significantly more toxic than the PARP inhibitor alone. In some aspects, the combination of the disclosure is not significantly more toxic than either the USP1 inhibitor alone or the PARP inhibitor alone.

In some aspects, the combination of the disclosure is less toxic than the PARP inhibitor alone. Accordingly, in some aspects, the present disclosure provides a method of treating cancer in a subject who previously received treatment with a first poly ADP-ribose polymerase (PARP) inhibitor, the method comprising administering to the subject a ubiquitin-specific-processing protease (USP1) inhibitor and a second PARP inhibitor, wherein the first and the second PARP inhibitors are the same or different PARP inhibitors. The treatment with the first PARP inhibitor may have been interrupted or discontinued, e.g., as a result of unacceptable toxicity and/or unacceptable adverse reactions. Exemplary toxicities or adverse reactions include hematological toxicity such as thrombocytopenia, anemia, or neutropenia, pneumonitis, dyspnea, fever, cough, wheezing, a radiological abnormality, hypertension, myelodysplastic syndrome/acute myeloid leukemia (MDS/AML), nausea, and/or fatigue.

In some aspects, the interruption of the treatment with the first PARP inhibitor was for at least one week, optionally from one week to four weeks. In some aspects, the interruption was for at least two weeks, optionally from two weeks to four weeks. In some aspects, the interruption was for at least three weeks, optionally from three weeks to four weeks. In some aspects, the interruption was for at least four weeks. In some aspects, the interruption was for no more than four weeks.

In some aspects, the dose of the first PARP inhibitor was reduced, for example, reduced to one quarter, one third, one half, two thirds, or three quarters of the dose prior to the reduction. The first PARP inhibitor can be olaparib, and the dose prior to the reduction can be 400 mg taken twice daily. Such a dose can be reduced, e.g., to 200 mg taken twice daily or 100 mg taken twice daily. The first PARP inhibitor can be niraparib, and the dose prior to the reduction can be 300 mg taken once daily. Such a dose can be reduced, e.g., to 200 mg taken once daily or 100 mg taken once daily. The first PARP inhibitor can be talazoparib, and the dose prior to the reduction can be 1 mg taken once daily. Such a dose can be reduced, e.g., to 0.75 mg taken once daily, 0.5 mg taken once daily, or 0.25 mg taken once daily. The first PARP inhibitor can be rucaparib, and the dose prior to the reduction can be 600 mg taken once daily. Such a dose can be reduced, e.g., to 500 mg taken twice daily, 400 mg taken twice daily, or 300 mg taken twice daily.

Various methods of treating diseases and disorders with the combinations of the disclosure are provided herein. Exemplary diseases and disorders that may be treated with the combinations of the disclosure include, but are not limited to, cancer.

In some aspects, methods of treating cancer with combinations of the disclosure are provided. Such methods comprise administering to a subject with cancer a therapeutically effective amount of a combination of the disclosure.

In some aspects, the cancer to be treated with a combination of the disclosure is selected from a hematological cancer, a lymphatic cancer, and a DNA damage repair pathway deficient cancer. In some aspects, the cancer to be treated with a combination of the disclosure is a cancer that comprises cancer cells with a mutation in a gene encoding p53. In some aspects, the cancer to be treated with a combination of the disclosure is a cancer that comprises cancer cells with a loss of function mutation in a gene encoding p53. In some aspects, the cancer to be treated with a combination of the disclosure is a cancer that comprises cancer cells with a mutation in a gene encoding BRCA1. In some aspects, the cancer to be treated with a combination of the disclosure is a cancer that comprises cancer cells with a mutation in a gene encoding BRCA2. In some aspects, the cancer to be treated with a combination of the disclosure is a cancer that comprises cancer cells with a loss of function mutation in a gene encoding ATM.

In some aspects, the cancer to be treated with a combination of the disclosure is selected from non-small cell lung cancer (NSCLC), osteosarcoma, ovarian cancer, and breast cancer. In some aspects, the cancer is uterine cancer. In some aspects, the cancer is peritoneal cancer. In some aspects, the cancer is endometrial cancer, In some aspects, the cancer is ovarian cancer or breast cancer. In some aspects, the cancer is ovarian cancer. In some aspects, the cancer is breast cancer. In some aspects, the cancer is a triple negative breast cancer. In some aspects, the cancer is an ovarian cancer. In some aspects, the ovarian cancer is a BRCA1 mutant cancer, a BRCA2 mutant cancer, or a p53 mutant cancer. In some aspects, the ovarian cancer is a BRCA1 mutant cancer and a p53 mutant cancer. In some aspects, the ovarian cancer is a BRCA1 and BRCA2 mutant cancer. In some aspects, the ovarian cancer is a BRCA2 mutant cancer.

In some aspects, the cancer to be treated with a combination of the disclosure is a cancer that comprises cancer cells with elevated levels of RAD51. The elevated levels of RAD51 can be elevated RAD51 protein levels, elevated RAD51 protein foci levels, and/or elevated levels RAD51 mRNA levels. In some aspects, a cancer that comprises cancer cells with elevated levels of RAD51 refers to a cancer wherein at least 10% of cells that are in the S/G2 phase of the cell cycle (e.g., geminin-positive cells) in a sample obtained from the cancer are RAD51-positive (e.g., contain 5 or more RAD51 nuclear foci).

A cancer with elevated levels of RAD51 can be a homologous-recombination deficient cancer. A cancer with elevated levels of RAD51 can be a BRCA1 mutant cancer, a BRCA2 mutant cancer, or a BRCA1 and BRCA2 mutant cancer. A cancer with elevated levels of RAD51 can be cancer with deleterious or suspected deleterious mutations in BRCA1 and BRCA2 genes and/or a positive Genomic Instability Score, e.g., as determined using myChoice® CDx (Myriad®).

In some aspects, the cancer to be treated with a combination of the disclosure is selected from the group consisting of bone cancer, including osteosarcoma and chondrosarcoma; brain cancer, including glioma, glioblastoma, astrocytoma, medulloblastoma, and meningioma; soft tissue cancer, including rhabdoid and sarcoma; kidney cancer; bladder cancer; skin cancer, including melanoma; and lung cancer, including non-small cell lung cancer; colon cancer, uterine cancer; nervous system cancer; head and neck cancer; pancreatic cancer; and cervical cancer. In some aspects, the cancer to be treated with a combination of the disclosure is selected from the group consisting of uterine cancer, peritoneal cancer, and endometrial cancer.

Various methods of treating cancer with a combination of the disclosure are provided herein. In some aspects, a therapeutically effective amount of a combination of the disclosure is administered to a subject with cancer.

In some aspects, such methods comprise (a) identifying a cancer in a subject as a USP1 and/or PARP inhibitor-sensitive cancer and then (b) administering a therapeutically effective amount of a combination of the disclosure to the subject.

In some aspects, such methods comprise administering to a subject with triple negative breast cancer a therapeutically effective amount of a combination of the disclosure.

In some aspects, a combination of the disclosure is used to treat a cancer, wherein the cancer is a homologous-recombination deficient cancer. In some aspects, a combination of the disclosure is used to treat a cancer, wherein the cancer comprises cancer cells with a mutation in a gene encoding p53. In some aspects, a combination of the disclosure is used to treat a cancer, wherein the cancer comprises cancer cells with a loss of function mutation in a gene encoding p53. In some aspects, a combination of the disclosure is used to treat a cancer that does not have a defect in the homologous recombination pathway.

In some aspects, a combination of the disclosure is used to treat a cancer, wherein the cancer is a BRCA1 mutant cancer. In some aspects, a combination of the disclosure is used to treat a cancer, wherein the cancer is a BRCA2 mutant cancer. In some aspects, a combination of the disclosure is used to treat a cancer, wherein the cancer is a BRCA1 mutant cancer and a BRCA2 mutant cancer. In some aspects, the cancer is not a BRCA1 mutant cancer or a BRCA2 mutant cancer. In some aspects, the cancer is a BRCA1 deficient cancer. In some aspects, the cancer is a BRCA2 deficient cancer. In some aspects, the cancer is a BRCA1 deficient cancer and a BRCA2 mutant cancer.

In some aspects, a combination of the disclosure is used to treat a cancer, wherein the cancer is an ATM mutant cancer. In some aspects, the cancer is not an ATM mutant cancer. In some aspects, the cancer is an ATM deficient cancer.

In some aspects, a combination of the disclosure is used to treat a cancer, wherein the cancer is a PARP inhibitor resistant or refractory cancer. In some aspects, a combination of the disclosure is used to treat a cancer, wherein the cancer is a PARP inhibitor resistant or refractory BRCA1-deficient cancer.

In some aspects, the cancer is a BRCA1 and/or BRCA2 mutant cancer, wherein the cancer comprises cells with elevated levels of RAD18, e.g., wherein the elevated levels of RAD18 are at least as high as the RAD18 protein and/or mRNA levels in ES2 cells (ES2 cells are publicly available, for example from the American Type Culture Collection (ATCC; CRL-1978)) or wherein the elevated levels of RAD18 are higher than the RAD18 protein and/or mRNA levels in HEP3B217 cells (HEP3B217 cells are publicly available, for example, from the ATCC (HB-8064)). In some aspects, a triple negative breast cancer is a BRCA1 and/or BRCA2 mutant cancer.

In some aspects, the cancer is a that comprises cancer cells with elevated levels of RAD51, e.g., elevated RAD51 protein levels, elevated RAD51 protein foci levels, and/or elevated RAD51 mRNA levels. In some aspects, a cancer that comprises cancer cells with elevated levels of RAD51 refers to a cancer wherein at least 10% of cells that are in the S/G2 phase of the cell cycle (e.g., geminin-positive cells) in a sample obtained from the cancer are RAD51-positive (e.g., contain 5 or more RAD51 nuclear foci).

In some instances, the cancer is a solid cancer. In some instances, the cancer is a hematological/lymphatic cancer. In some instances, the cancer is a DNA damage repair pathway deficient cancer. In some instances, the cancer is a homologous-recombination deficient cancer. In some instances, the cancer comprises cancer cells with a mutation in a gene encoding p53. In some instances, the cancer comprises cancer cells with a loss of function mutation in a gene encoding p53. In some instances, the cancer is selected from the group consisting of non-small cell lung cancer (NSCLC), osteosarcoma, ovarian cancer, and breast cancer (including triple negative breast cancer). In some instances, the cancer is ovarian cancer or breast cancer (including triple negative breast cancer). In some instances, the cancer is ovarian cancer. In some instances, the cancer is breast cancer (including triple negative breast cancer.) In some instances, the cancer is uterine cancer. In some instances, the cancer is peritoneal cancer. In some instances, the cancer is endometrial cancer.

In some aspects, a combination of the disclosure is used in combination with one or more additional therapeutic agents to treat cancer.

In some aspects, provided herein are combinations of the disclosure for use as a medicament or for use in preparing a medicament, e.g., for the treatment of cancer. In some aspects, provided herein are combinations of the disclosure for use in a method for the treatment of cancer.

In some aspects, methods of treating cancers comprising cells with elevated levels of RAD51 are provided. Cancers comprising cells with elevated levels of RAD51 can be referred to herein as “RAD51 high cancers.” Such methods comprise administering to a subject with a RAD51 high cancer a therapeutically effective amount of a USP1 inhibitor or a combination of a USP1 inhibitor and a PARP inhibitor.

In some aspects, the RAD51 high cancer to be treated with a USP1 inhibitor or a combination of a USP1 inhibitor and a PARP inhibitor is selected from a hematological cancer, a lymphatic cancer, and a DNA damage repair pathway deficient cancer. In some aspects, the RAD51 high cancer to be treated with a USP1 inhibitor or a combination of a USP1 inhibitor and a PARP inhibitor is a homologous-recombination deficient cancer.

In some aspects, the RAD51 high cancer to be treated with USP1 inhibitor or a combination of a USP1 inhibitor and a PARP inhibitor is selected from non-small cell lung cancer (NSCLC), osteosarcoma, ovarian cancer, and breast cancer. In some aspects, the cancer is uterine cancer. In some aspects, the RAD51 high cancer is peritoneal cancer. In some aspects, the RAD51 high cancer is endometrial cancer. In some aspects, the RAD51 high cancer is ovarian cancer or breast cancer. In some aspects, the RAD51 high cancer is ovarian cancer. In some aspects, the RAD51 high cancer is breast cancer. In some aspects, the RAD51 high cancer is a triple negative breast cancer. In some aspects, the RAD51 high cancer is an ovarian cancer.

In some aspects, the RAD51 high cancer to be treated with USP1 inhibitor or a combination of a USP1 inhibitor and a PARP inhibitor is selected from the group consisting of bone cancer, including osteosarcoma and chondrosarcoma; brain cancer, including glioma, glioblastoma, astrocytoma, medulloblastoma, and meningioma; soft tissue cancer, including rhabdoid and sarcoma; kidney cancer; bladder cancer; skin cancer, including melanoma; and lung cancer, including non-small cell lung cancer; colon cancer, uterine cancer; nervous system cancer; head and neck cancer; pancreatic cancer; and cervical cancer. In some aspects, the RAD51 high cancer to be treated with a USP1 inhibitor or a combination of a USP1 inhibitor and a PARP inhibitor is selected from the group consisting of uterine cancer, peritoneal cancer, and endometrial cancer.

Various methods of treating a RAD51 high cancer with a combination of the disclosure are provided herein. In some aspects, a therapeutically effective amount of a combination of the disclosure is administered to a subject with a RAD51 high cancer.

In some aspects, such methods comprise (a) detecting levels of RAD51 (e.g., RAD51 protein and/or RAD51 mRNA) in cancer cells (e.g., in a cancer sample obtained from the subject) and then (b) administering a therapeutically effective amount of a USP1 inhibitor to a subject have a cancer comprising cells with elevated levels of RAD51. In some aspects, such methods comprise (a) detecting levels of RAD51 (e.g., RAD51 protein and/or RAD51 mRNA) in cancer cells (e.g., in a cancer sample obtained from the subject) and then (b) administering a USP1 inhibitor in combination with a PARP inhibitor to a subject have a cancer comprising cells with elevated levels of RAD51.

### Pharmaceutical Combination Compositions

Combinations of the disclosure can be administered to a mammal in the form of a raw chemicals without any other components present, or combinations of the disclosure can also be administered to a mammal as part of a pharmaceutical composition containing the compound combined with a suitable pharmaceutically acceptable carrier (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Such a carrier can be selected from pharmaceutically acceptable excipients and auxiliaries. The term “pharmaceutically acceptable carrier” or “pharmaceutically acceptable vehicle” encompasses any of the standard pharmaceutical carriers, solvents, surfactants, or vehicles. Standard pharmaceutical carriers and their formulations are described in Remington's Pharmaceutical Sciences, Mack Publishing Co., Easton, Pa., 19th ed. 1995.

A pharmaceutical combination composition of the present disclosure may be prepared as liquid suspensions or solutions using a liquid, such as an oil, water, an alcohol, and combinations of these.

The pharmaceutical combination compositions to be used for in vivo administration can be sterile. This is readily accomplished by filtration through, e.g., sterile filtration membranes.

Pharmaceutical combination compositions within the scope of the present disclosure include all compositions where a USP1 inhibitor and a PARP inhibitor of the disclosure are combined with one or more pharmaceutically acceptable carriers. In one embodiment, the USP1 inhibitor and PARP inhibitor of the disclosure are present in the composition in an amount that is effective to achieve its intended therapeutic purpose.

A pharmaceutical combination composition of the present disclosure can be administered to any patient that may experience the beneficial effects of a combination of the disclosure. Foremost among such patients are mammals, e.g., humans and companion animals, although the disclosure is not intended to be so limited. In one aspect, the patient is a human. In another aspect, a pharmaceutical combination composition of the present disclosure can be administered to a patient having PARP inhibitor resistant or refractory cancer. In another embodiment, a pharmaceutical combination composition of the present disclosure can be administered to a patient having PARP inhibitor resistant or refractory BRCA1-deficient cancer. In another embodiment, a pharmaceutical combination composition of the present disclosure can be administered to a patient having a cancer that comprises cancer cells with elevated levels of RAD51, e.g., elevated RAD51 protein levels, elevated RAD51 protein foci levels, and/or elevated RAD51 mRNA levels. In some aspects, a pharmaceutical combination composition of the present disclosure can be administered to a patient having a cancer wherein at least 10% of cells that are in the S/G2 phase of the cell cycle (e.g., geminin-positive cells) in a sample obtained from the cancer are RAD51-positive (e.g., contain 5 or more RAD51 nuclear foci).

In another embodiment, the present disclosure provides kits that comprise a combination of the disclosure packaged in a manner that facilitates their use to practice methods of the present disclosure. In one embodiment, the kit includes a USP1 inhibitor and a PARP inhibitor of the disclosure packaged in a container, such as a sealed bottle or vessel, with a label affixed to the container or included in the kit that describes use of the compounds to practice the methods of the disclosure. In one embodiment, the combination composition is packaged in a unit dosage form. The kit further can include a device suitable for administering the combination composition according to the intended route of administration. In some aspects, the present disclosure provides a kit that comprises a USP1 inhibitor and a PARP inhibitor of the disclosure, or a pharmaceutically acceptable salt or solvate thereof, and instructions for administering the compounds, or pharmaceutically acceptable salts or solvates thereof, to a patient having cancer.

In some aspects, the present disclosure provides a pharmaceutical combination composition comprising a USP1 inhibitor and a PARP inhibitor of the disclosure, or a pharmaceutically acceptable salt or solvate thereof, and a pharmaceutically acceptable carrier.

In some aspects, the present disclosure provides a pharmaceutical combination composition comprising a USP1 inhibitor and a PARP inhibitor of the disclosure, or a pharmaceutically acceptable salt or solvate thereof, and a pharmaceutically acceptable carrier, wherein the combination binds to a protein encoded by the USP1 gene and/or a PARP gene.

In some aspects, the present disclosure provides a pharmaceutical combination composition comprising a USP1 inhibitor and a PARP inhibitor of the disclosure, or a pharmaceutically acceptable salt or solvate thereof, and a pharmaceutically acceptable carrier, wherein the pharmaceutical composition is for use in treating cancer.

In some aspects, the present disclosure provides a pharmaceutical combination composition comprising a USP1 inhibitor and a PARP inhibitor of the disclosure, or a pharmaceutically acceptable salt or solvate thereof, and a pharmaceutically acceptable carrier, wherein the pharmaceutical composition is for the manufacture of a medicament for treatment of cancer.

### EXAMPLES

### Example 1: In Vitro Assays

### Colony Formation Assay

In vitro experiments were conducted using the colony formation unit (CFU) assay on various cell lines. The CFU assay involved first establishing what cell plating density enabled the development of clearly interspersed colonies on a six-well plate when left to grow for around 14 days. Once this density had been identified, cells were plated on day −1 and on day 0, the wells were treated with DMSO or increasing concentrations of USP1 inhibitor or Niraparib (3 nM, 10 nM, 30 nM, 100 nM, and 300 nM), or increasing concentrations of USP1 inhibitor or Olaparib (3 nM, 10 nM, 30 nM, 100 nM, and 300 nM). Media was changed on day 8 containing appropriate concentrations of DMSO, USP1 inhibitor, Niraparib, or Olaparib. At or around day 14 when clearly interspersed colonies were visible in the DMSO treated wells, the cells were fixed and stained using 0.1% crystal violet in 10% ethanol for 20 minutes at room temperature. The plates were imaged then the amount of crystal violet stain in each well was quantified by extracting the crystal violet into 10% acetic acid and the absorbance measured at 565 nm. The CFU results are shown in Table 1 and Table 2.

TABLE 1

| | | Number | ATM | BRCA1 | BRCA2 | TP53 | Formula | | |
|---|---|---|---|---|---|---|---|---|---|
| | | of | manual | manual | manual | automatic | II IC50 | Niraparib | Bliss |
| Cell Line | Organ | Repeats | call | call | call | call | (nM) | IC50 (nM) | Score |
| HS695T | skin | 1 | Loss of | | | | >300 | 36.9 | N/A |
| | | | function | | | | | | |
| BICR6 | head and neck | 1 | | | | Loss of | >300 | 149 | N/A |
| | | | | | | function | | | |
| 143B | bone | 1 | | | | | >300 | >300 | N/A |
| 22RV1 | prostate | 1 | | | | Loss of | >300 | >300 | N/A |
| | | | | | | function | | | |
| ASPC1 | pancreas | 1 | | | | Loss of | >300 | >300 | N/A |
| | | | | | | function | | | |
| BICR56 | head and neck | 1 | | | | Loss of | >300 | >300 | N/A |
| | | | | | | function | | | |
| BT474 | breast | 1 | | | Possible | | >300 | >300 | N/A |
| | | | | | loss of | | | | |
| | | | | | function | | | | |
| ECGI10 | esophagus | 1 | | | | | >300 | >300 | N/A |
| HCC95 | lung | 1 | | | | | >300 | >300 | N/A |
| HS821T | bone | 1 | | | | | >300 | >300 | N/A |
| HS934T | melanoma | 1 | | | | | >300 | >300 | N/A |
| HUO3N1 | bone | 1 | | | | Loss of | >300 | >300 | N/A |
| | | | | | | function | | | |
| MG63 | bone | 1 | | | | | >300 | >300 | N/A |
| NCIH1648 | lung | 1 | | | | Loss of | >300 | >300 | N/A |
| | | | | | | function | | | |
| NCIH1838 | lung | 1 | Possible | | | | >300 | >300 | N/A |
| | | | loss of | | | | | | |
| | | | function | | | | | | |
| NCIH838 | lung | 1 | | | | | >300 | >300 | N/A |
| OE19 | esophagus | 1 | | | | Loss of | >300 | >300 | N/A |
| | | | | | | function | | | |
| OVK18 | ovary | 1 | | | | Loss of | >300 | >300 | N/A |
| | | | | | | function | | | |
| SNGM | uterus | 2 | WT | | | | >300 | >300 | N/A |
| SNU1076 | head and neck | 1 | | | | Loss of | >300 | >300 | N/A |
| | | | | | | function | | | |
| SNU213 | pancreas | 1 | | | | | >300 | >300 | N/A |
| T174 | breast | 1 | | | | | >300 | >300 | N/A |
| TCCSUP | bladder | 1 | | | | | >300 | >300 | N/A |
| VMRCRCZ | kidney | 1 | | | Possible | Loss of | >300 | >300 | N/A |
| | | | | | loss of | function | | | |
| | | | | | function | | | | |
| MSTO211H | lung | 1 | | | | | >300 | >300 | 25.4 |
| NCIH520 | lung | 1 | | | | | >300 | >300 | 20.3 |
| SUM149PT | breast | 1 | | Loss of | | | >300 | 135 | 18.1 |
| | | | | function | | | | | |
| DOTC24510 | cervix | 1 | | | | | >300 | 30.4 | 18 |
| JHOS2 | ovary | 1 | | | | | >300 | >300 | 17.8 |
| SNUC4 | colon | 1 | | Possible | | | >300 | >300 | 17.4 |
| | | | | loss of | | | | | |
| | | | | function | | | | | |
| HCC1806 | breast | 2 | | | | Loss of | 114 | 62.9 | 16.7 |
| | | | | | | function | | | |
| COV362 | ovary | 3 | Possible | Possible | | | >300 | 177 | 16.3 |
| | | | loss of | loss of | | | | | |
| | | | function | function | | | | | |
| RERFLCAI | lung | 1 | | | | | >300 | >300 | 16.1 |
| CW2 | colon | 2 | | | Possible | | >300 | >300 | 15.3 |
| | | | | | loss of | | | | |
| | | | | | function | | | | |
| BICR78 | head and neck | 1 | | | | Loss of | >300 | >300 | 15 |
| | | | | | | function | | | |
| GOTO | nervous | 1 | | | | | >300 | 22.4 | 14.2 |
| | system | | | | | | | | |
| SNU668 | stomach | 1 | | Possible | | | >300 | >300 | 13.4 |
| | | | | loss of | | | | | |
| | | | | function | | | | | |
| TE11 | esophagus | 2 | | | | | 134 | 176 | 13.1 |
| HCC2108 | lung | 1 | | | | Loss of | >300 | 95.5 | 12.8 |
| | | | | | | function | | | |
| HCT116 | colon | 2 | | | Possible | | 110 | >300 | 12.7 |
| | | | | | loss of | | | | |
| | | | | | function | | | | |
| UWB1289BRCA1 | ovary | 1 | | | | | 139 | 186 | 12.3 |
| HPAC | pancreas | 1 | | | | Loss of | >300 | >300 | 12.2 |
| | | | | | | function | | | |
| HBCx8 | breast | 1 | | | | | >300 | 107 | 12.1 |
| SKES1 | bone | 2 | | | | | >300 | 23.6 | 11.9 |
| SNU119 | ovary | 1 | | | | | >300 | 39 | 11.3 |
| SNU407 | colon | 1 | | | Possible | Loss of | >300 | 72.8 | 11.3 |
| | | | | | loss of | function | | | |
| | | | | | function | | | | |
| A2058 | skin | 1 | | | | | >300 | 58.4 | 11.2 |
| NCIH1915 | lung | 2 | | | | Loss of | 52.2 | 66.2 | 10.5 |
| | | | | | | function | | | |
| KM12 | colon | 1 | | | Possible | Loss of | 220 | 106 | 10.5 |
| | | | | | loss of | function | | | |
| | | | | | function | | | | |
| SUIT2 | pancreas | 1 | | | | | >300 | >300 | 9.98 |
| COV434 | ovary | 1 | | | | | >300 | 106 | 9.79 |
| JIMT1 | breast | 1 | | | | | 16.6 | 25.3 | 9.34 |
| HCC1395 | breast | 3 | | Loss of | | | >300 | 8.26 | 18 |
| | | | | function | | | | | |
| PK45H | pancreas | 1 | | | | | 294 | >300 | 8.17 |
| SKOV3_BRCA1null | ovary | 1 | | | | | >300 | 262 | 8.11 |
| HCC1954 | breast | 2 | | Possible | | | >300 | >300 | 4 |
| | | | | loss of | | | | | |
| | | | | function | | | | | |
| HEC151 | endometrial | 1 | | | | | >300 | >300 | 7.76 |
| CHAGOK1 | lung | 1 | | | | | >300 | >300 | 7.72 |
| NCIH1573 | lung | 1 | | | | | 227 | 15.2 | 7.47 |
| HT115 | colon | 1 | | Possible | | Loss of | 150 | 15.6 | 7.45 |
| | | | | loss of | | function | | | |
| | | | | function | | | | | |
| LU99 | lung | 1 | | | | | 35.4 | 88.6 | 7.23 |
| RKO | colon | 1 | | | Possible | | >300 | >300 | 7.15 |
| | | | | | loss of | | | | |
| | | | | | function | | | | |
| HPAFII | pancreas | 2 | | | | | >300 | >300 | 6.99 |
| HCC1438 | lung | 1 | | | | Loss of | >300 | 42.5 | 6.98 |
| | | | | | | function | | | |
| LNCAP | prostate | 1 | | | | | >300 | >300 | 6.87 |
| A2780 | ovary | 1 | | | | | >300 | >300 | 6.77 |
| NCIH1651 | lung | 1 | | | | | 85.7 | 32.4 | 6.7 |
| FADU | head and neck | 2 | | | Possible | Loss of | >300 | >300 | 6.67 |
| | | | | | loss of | function | | | |
| | | | | | function | | | | |
| NCIH1373 | lung | 2 | | | | Loss of | >300 | >300 | 6.43 |
| | | | | | | function | | | |
| UWB1289 | ovary | 4 | | Loss of | | Loss of | 22.3 | 28.4 | 6.41 |
| | | | | function | | function | | | |
| SNU81 | colon | 1 | | | Loss of | Loss of | 10.2 | 18.6 | 6.25 |
| | | | | | function | function | | | |
| KU1919 | bladder | 2 | | | | | 25 | 181 | 6.16 |
| NCIH1693 | lung | 4 | | | Possible | Loss of | 12.7 | >300 | 5.96 |
| | | | | | loss of | function | | | |
| | | | | | function | | | | |
| KYSE270 | esophagus | 3 | | | | Loss of | >300 | 16.9 | 5.86 |
| | | | | | | function | | | |
| UBLC1 | bladder | 1 | | | | | >300 | >300 | 5.72 |
| HCT15 | colon | 1 | | | Loss of | Loss of | >300 | >300 | 5.7 |
| | | | | | function | function | | | |
| SKOV3 | ovary | 2 | WT | | | Loss of | >300 | >300 | 5.62 |
| | | | | | | function | | | |
| HSC2 | head and neck | 3 | | | | Loss of | >300 | >300 | 5.5 |
| | | | | | | function | | | |
| HCC202 | breast | 1 | | | Possible | Loss of | >300 | 103 | 5.41 |
| | | | | | loss of | function | | | |
| | | | | | function | | | | |
| OVMANA | ovary | 1 | | | | | >300 | >300 | 5.39 |
| HOS | bone | 1 | | | | | >300 | >300 | 5.35 |
| VMRCRCW | kidney | 1 | | | | Loss of | >300 | >300 | 5.27 |
| | | | | | | function | | | |
| RT11284 | bladder | 1 | | | | | >300 | >300 | 5.11 |
| HCC1569 | breast | 1 | | | WT | Loss of | >300 | 97.3 | 4.76 |
| | | | | | | function | | | |
| KS1 | nervous system | 1 | WT | | | | >300 | >300 | 4.65 |
| PK8 | pancreas | 1 | | | | | >300 | >300 | 4.57 |
| KYSE410 | esophagus | 1 | | | | | >300 | >300 | 4.29 |
| HBCx6 | breast | 1 | | | | | >300 | 30.4 | 4.28 |
| HBCx9 | breast | 1 | | | | | >300 | 55.8 | 4.1 |
| OVSAHO | ovary | 2 | | | | Loss of | >300 | >300 | 3.91 |
| | | | | | | function | | | |
| HT144 | skin | 2 | Loss of | | | | 84.3 | 114 | 3.84 |
| | | | function | | | | | | |
| RT112 | bladder | 1 | | | | | >300 | >300 | 3.82 |
| TGBC1TKB | biliary tract | 1 | | | | Loss of | >300 | >300 | 3.82 |
| | | | | | | function | | | |
| SKOV3_ATMnull | ovary | 1 | | | | | >300 | >300 | 3.81 |
| HBCx17 | breast | 1 | | | | | >300 | 13.8 | 3.69 |
| DMS53 | lung | 1 | | | | | >300 | 168 | 3.62 |
| CCK81 | colon | 1 | | | | | >300 | >300 | 3.46 |
| 647V | bladder | 2 | Possible | | | Loss of | 33.9 | 96.7 | 3.37 |
| | | | loss of | | | function | | | |
| | | | function | | | | | | |
| YKG1 | nervous system | 1 | | | | | >300 | >300 | 3.15 |
| HMC18 | breast | 1 | | | | Loss of | >300 | >300 | 3.09 |
| | | | | | | function | | | |
| HCC1500 | breast | 1 | Loss of | | | | >300 | 61.1 | 2.78 |
| | | | function | | | | | | |
| HCC1937 | breast | 2 | | | | Loss of | >300 | 244 | 2.76 |
| | | | | | | function | | | |
| TE9 | esophagus | 1 | | | WT | Loss of | >300 | >300 | 2.24 |
| | | | | | | function | | | |
| NOS1 | bone | 1 | | | | | >300 | >300 | 2.13 |
| SNU489 | nervous system | 1 | | | | Loss of | >300 | >300 | 2.11 |
| | | | | | | function | | | |
| SKOV3_OR1a1null | ovary | 1 | | | | | >300 | >300 | 1.95 |
| CAOV3 | ovary | 2 | Possible | | | Loss of | 27.8 | 241 | 1.72 |
| | | | loss of | | | function | | | |
| | | | function | | | | | | |
| SN12C | kidney | 1 | | | | Loss of | 64.7 | >300 | 1.62 |
| | | | | | | function | | | |
| SNU626 | nervous system | 1 | | | | Loss of | >300 | >300 | 1.28 |
| | | | | | | function | | | |
| U2OS | bone | 1 | | | | | >300 | >300 | 1.2 |
| MDAMB231 | breast | 2 | | | | | >300 | >300 | 1.18 |
| SAOS2 | bone | 1 | | | | | >300 | 201 | 1.16 |
| SJSA1 | bone | 1 | | | | | >300 | >300 | 1.14 |
| PECAPJ49 | head and neck | 1 | | | | | >300 | >300 | 1.01 |
| UMUC3 | bladder | 2 | Possible | | | | >300 | >300 | 0.963 |
| | | | loss of | | | | | | |
| | | | function | | | | | | |
| SCC15 | head and neck | 1 | | | | Loss of | 28 | >300 | 0.8 |
| | | | | | | function | | | |
| CAPAN2 | pancreas | 1 | | | | Loss of | >300 | >300 | 0.487 |
| | | | | | | function | | | |
| HT1080 | soft tissue | 1 | | | | | >300 | >300 | 0.272 |
| KYSE140 | esophagus | 1 | | | | | >300 | >300 | 0.262 |
| KP3 | pancreas | 1 | | | | Loss of | >300 | >300 | 0.101 |
| | | | | | | function | | | |
| KURAMOCHI | ovary | 1 | | | Possible | | >300 | >300 | 0 |
| | | | | | loss of | | | | |
| | | | | | function | | | | |
| CFPAC1 | pancreas | 1 | | | | | >300 | >300 | −0.196 |
| GB1 | nervous system | 1 | | | | | >300 | >300 | −0.279 |
| NCIH1299 | lung | 2 | | | | | >300 | >300 | −0.395 |
| SNU8 | ovary | 1 | | | | Loss of | >300 | >300 | −0.397 |
| | | | | | | function | | | |
| LUDLU1 | lung | 1 | | | | | >300 | >300 | −0.536 |
| CAPAN1 | pancreas | 4 | | | Possible | | >300 | 185 | −1 |
| | | | | | loss of | | | | |
| | | | | | function | | | | |
| OV56 | ovary | 2 | | | | Loss of | >300 | >300 | −0.581 |
| | | | | | | function | | | |
| RL952 | uterus | 1 | | | Possible | Loss of | >300 | >300 | −0.807 |
| | | | | | loss of | function | | | |
| | | | | | function | | | | |
| ES2 | ovary | 3 | | | | | 27.5 | >300 | −0.904 |
| TE5 | esophagus | 1 | | | | | >300 | >300 | −0.91 |
| HSC3 | head and neck | 1 | | | | Loss of | >300 | >300 | −0.935 |
| | | | | | | function | | | |
| MDAMB436 | breast | 12 | | Loss of | | Loss of | 6.46 | 4.73 | −0.986 |
| | | | | function | | function | | | |
| KALS1 | nervous system | 1 | | | | | >300 | >300 | −1.03 |
| JHH7 | liver | 1 | | | | | >300 | >300 | −1.07 |
| HEC1B | uterus | 1 | | | | | >300 | >300 | −1.47 |
| PECAPJ15 | head and neck | 1 | | | | | >300 | >300 | −1.48 |
| AN3CA | uterus | 1 | | | | Loss of | >300 | 101 | −1.93 |
| | | | | | | function | | | |
| SKMEL24 | skin | 1 | Possible | | | | >300 | >300 | −1.93 |
| | | | loss of | | | | | | |
| | | | function | | | | | | |
| T226 | breast | 1 | | | | | >300 | >300 | −1.97 |
| QGP1 | pancreas | 1 | | | | Loss of | >300 | >300 | −2.03 |
| | | | | | | function | | | |
| COV504 | ovary | 1 | | | | Loss of | 6.63 | 4.17 | −2.13 |
| | | | | | | function | | | |
| HBCx3 | breast | 1 | | | | | >300 | 20.8 | −2.41 |
| YD38 | head and neck | 1 | | | | Loss of | >300 | >300 | −2.42 |
| | | | | | | function | | | |
| TE14 | esophagus | 1 | | | Possible | Loss of | >300 | >300 | −3.41 |
| | | | | | loss of | function | | | |
| | | | | | function | | | | |
| SNUC5 | colon | 1 | Possible | | | | >300 | 92.4 | −3.77 |
| | | | loss of | | | | | | |
| | | | function | | | | | | |
| RCM1 | colon | 1 | | | | Loss of | >300 | >300 | −4.06 |
| | | | | | | function | | | |
| KLE | uterus | 1 | | | | | >300 | >300 | −4.23 |
| 59M | ovary | 1 | Possible | | | Loss of | 34.9 | 63.4 | −4.47 |
| | | | loss of | | | function | | | |
| | | | function | | | | | | |
| BICR18 | head and neck | 1 | | | | Loss of | >300 | >300 | −4.48 |
| | | | | | | function | | | |
| 8305C | thyroid | 1 | | | | | >300 | >300 | −5.09 |
| HBCx19 | breast | 1 | | | | | >300 | >300 | −5.22 |
| KMBC2 | bladder | 1 | | | | | >300 | >300 | −5.4 |
| SCC25 | head and neck | 1 | | | | Loss of | >300 | >300 | −5.55 |
| | | | | | | function | | | |
| G292CLONEA141B1 | bone | 1 | | | | Loss of | >300 | >300 | −5.91 |
| | | | | | | function | | | |
| HS870T | bone | 1 | | | | | >300 | >300 | −5.91 |
| GSU | stomach | 2 | | | | | >300 | >300 | −5.99 |
| U251MG | nervous system | 1 | | | | | >300 | >300 | −6.15 |
| SNU1041 | head and neck | 1 | Possible | | | | >300 | >300 | −6.88 |
| | | | loss of | | | | | | |
| | | | function | | | | | | |
| HS739T | breast | 1 | | | | | >300 | >300 | −7.43 |
| BICR22 | head and neck | 1 | | | | Loss of | >300 | >300 | −7.51 |
| | | | | | | function | | | |
| OVA2BUR | ovary | 1 | | | | | >300 | >300 | −8.68 |
| HBCx13B | breast | 1 | | | | | >300 | >300 | −8.98 |
| HBCx2 | breast | 1 | | | | | >300 | >300 | −10.4 |
| SW948 | colon | 2 | Possible | | | Loss of | >300 | >300 | −10.7 |
| | | | loss of | | | function | | | |
| | | | function | | | | | | |
| HUO9 | bone | 1 | | | | | >300 | >300 | −11.1 |
| HS578T | breast | 1 | | | | | >300 | >300 | −13.8 |
| HS888T | bone | 1 | | | | | >300 | >300 | −14.5 |

TABLE 2

| | | Number | ATM | BRCA1 | BRCA2 | TP53 | Formula | | |
|---|---|---|---|---|---|---|---|---|---|
| | | of | manual | manual | manual | automatic | I IC50 | Olaparib | Bliss |
| Cell Line | Organ | Repeats | call | call | call | call | (nM) | IC50 (nM) | Score |
| MDAMB436 | breast | 12 | | Loss of | | Loss of | 4.5 | 0.1 | −4.27 |
| | | | | function | | function | | | |
| TE11 | esophagus | 1 | | | | | >316 | >316 | −10.5 |
| MER014 | pleura | | | | | | >316 | >316 | −2.15 |
| MER082 | pleura | | | | | | >316 | >316 | −3.95 |
| CW2 | colon | 1 | | | Possible | | 67 | >316 | −5.65 |
| | | | | | loss of | | | | |
| | | | | | function | | | | |
| UWB1289BRCA1 | ovary | 1 | | | | | 113 | >316 | 9.29 |
| HPAC | pancreas | 1 | | | | Loss of | >316 | >316 | −6.71 |
| | | | | | | function | | | |
| HCC1806 | breast | 2 | | | | Loss of | 62 | 97 | 16.7 |
| | | | | | | function | | | |
| NCIH520 | lung | | | | | | >316 | >316 | 19.5 |
| MDAMB436 | breast | 12 | | Loss of | | Loss of | 4.5 | 0.1 | −4.27 |
| | | | | function | | function | | | |
| TE11 | esophagus | 1 | | | | | >316 | >316 | −10.5 |
| MER014 | pleura | | | | | | >316 | >316 | −2.15 |
| MER082 | pleura | | | | | | >316 | >316 | −3.95 |
| CW2 | colon | 1 | | | Possible | | 67 | >316 | −5.65 |
| | | | | | loss of | | | | |
| | | | | | function | | | | |
| COV362 | ovary | | Possible | Possible | | | 203 | 17 | 4.96 |
| | | | loss of | loss of | | | | | |
| | | | function | function | | | | | |
| HCT116 | colon | 1 | | | Possible | | >316 | >316 | 28.8 |
| | | | | | loss of | | | | |
| | | | | | function | | | | |
| MSTO211H | lung | 1 | | | | | >316 | >316 | 18.2 |
| JHOS2 | ovary | 1 | | | | | | | |
| SNU668 | stomach | 1 | | Possible | | | | | |
| | | | | loss of | | | | | |
| | | | | function | | | | | |
| GOTO | nervous | 1 | | | | | | | |
| | system | | | | | | | | |
| SNUC4 | colon | 1 | | Possible | | | | | |
| | | | | loss of | | | | | |
| | | | | function | | | | | |
| HCC2108 | lung | 1 | | | | Loss of | | | |
| | | | | | | function | | | |
| SUM149PT | breast | 1 | | Loss of | | | | | |
| | | | | function | | | | | |
| JIMT1 | breast | 1 | | | | | | | |

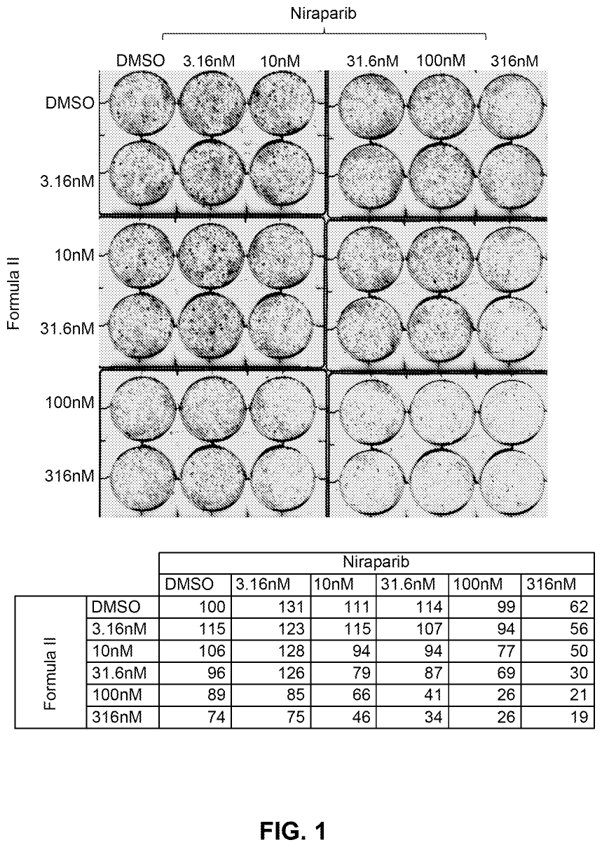

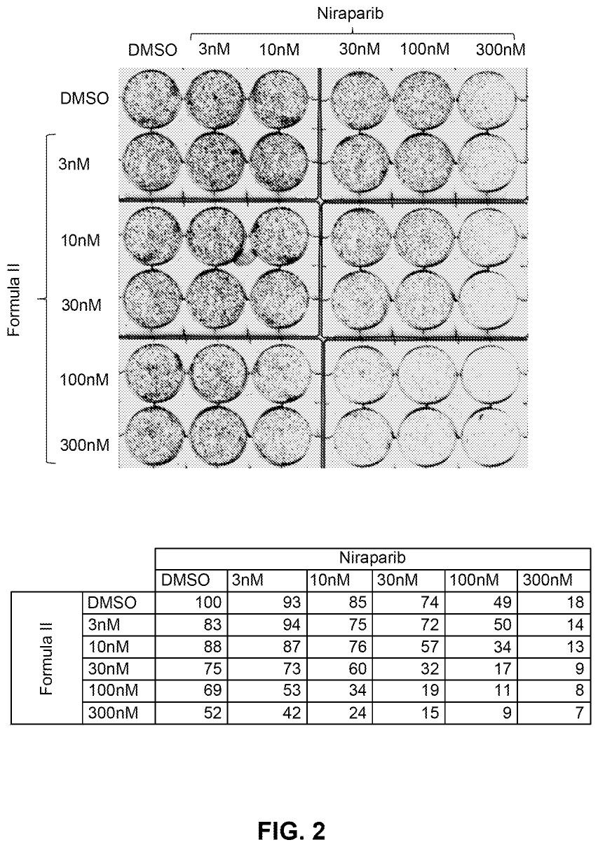

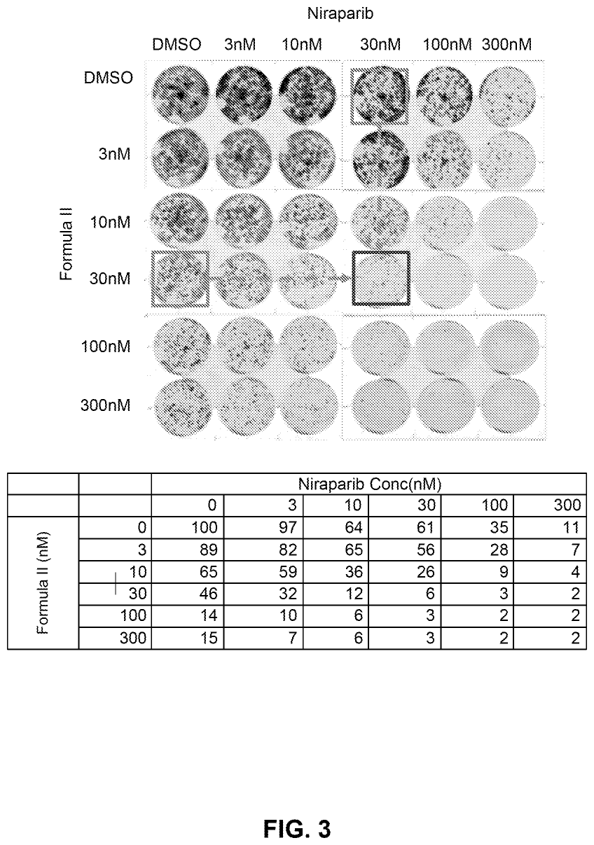

FIGS. 1, 2, and 3 show representative results from the colony formation assays using the USP1 inhibitor of Formula II. FIG. 1 shows the synergistic effect of combining a USP1 inhibitor of Formula II and Niraparib. In the JHOS2 cell line shown in FIG. 1, USP1 inhibitor and Niraparib had little to no activity as single agents up to 300 nM; however, the combination of both agents led to a synergistic effect on cell growth. Additionally, combining 100 nM of each agent had greater effects than 300 nM of Niraparib alone. In the COV362 cell line shown in FIG. 2, the USP1 inhibitor of Formula II and Niraparib had modest activity as single agents; but in combination, the USP1 inhibitor and Niraparib had a synergistic effect on cell growth. Additionally, combining 100 nM of each agent showed greater effects than 300 nM of Niraparib alone. FIG. 3 depicts the synergistic activity observed for UWB 1.289, a BRCA1 mutant ovarian cell line. Although UWB1.289 was sensitive to both a USP1 inhibitor and Niraparib as single agents, the combination of 30 nM of each agent had equivalent growth effects as 300 nM Niraparib alone.

The results in Table 1 showed synergy was detected in cell lines having an enrichment for BRCA1 loss of function mutations or possible loss of function mutations, suggesting that patients with such mutations may benefit from a combination therapy of a USP1 inhibitor and a PARP inhibitor. For example, out the total number of BRCA1 mutant cell lines ran in the CFU assays, 8 out of 9 cell lines showed synergy above a cutoff score of 6.

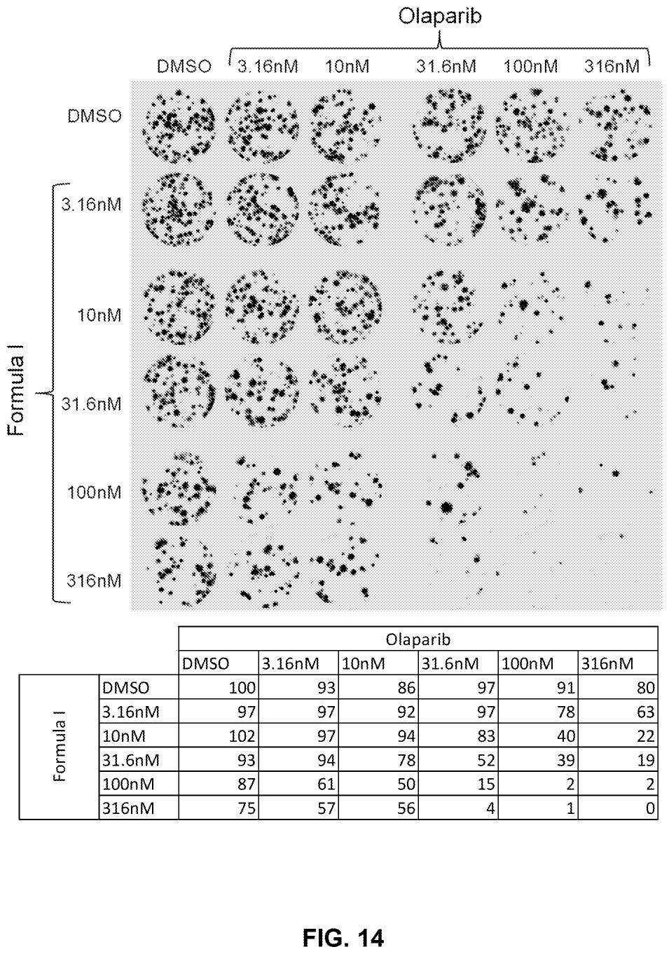

FIG. 14 shows representative results from the colony formation assay using the USP1 inhibitor of Formula I in HCT 116 ovarian cancer cells. The results in Table 2 showed synergy was detected in ovarian, breast, lung, and colon cancer cells.

### Determination of IC₅₀ Values and Synergy Scores

IC50 values were calculated by fitting a two-parameter hill equation to the dose-response measurements. Non-linear-least-squares was used to find parameter values that minimize the squared error of the model fit to measured dose response. Non-linear-least-squares estimation was performed using the minpack.lm R package, version 1.2-1. Bliss synergy scores were calculated using the synergyfinder R package version 1.6.1.

### Determination of Mutation Status

Mutation in ATM, BRCA1, and BRCA2 were determined using an in-house pipeline. CCLE RNA-seq data was analyzed by the GATK tool MuTect2 version 3.7-0-g56f2c1a, to identify variants, then classified using the GATK tool Funcotator version 3.7-0-g56f2c1a. Funcotator classified variant calls as one of “silent”, “missense”, “splice-site”, “non-sense”, or “frameshift”. An automatic mutation that was classified as splice-site, non-sense or frameshift mutations were manually reviewed. The manual review assessed whether the mutation was homozygous, and whether the call could be attributed to sequencing or variant-calling artifacts such as low sequencing depth or indels located in homopolymer sequences, and summarized the impact on the gene when multiple events were called for a single gene. The output of the manual mutation reviews was a classification of the impact on gene function as one of “loss-of-function”, “possible-loss-of-function”, or “wild-type”. The mutation calls for TP53 were extracted from the CCLE_mutations.csv file, downloaded from depmap.org.

### Example 2: PDX Model Selection

Five patient-derived xenograft models were selected based on availability and with a variety of BRCA and HRD mutational signatures. PARP inhibitor (PARPi) activity was known in selected models based on historical clinical data and from internally-generated data from XenTech SAS. Based on this historical data, a range of PARPi responsive and non-responsive models were chosen. Table 3 shows a summary of the models chosen for testing, single agent activity with the compound of Formula I, and combination activity with the compound of Formula I and Olaparib.

TABLE 3

| | Tumor | BRCA | HRD Score | Formula I | Combo |
|---|---|---|---|---|---|
| | Model | Mutation | (Myriad)* | Sensitive | Activity |
| PARP | HBCx-10 | 2 | 57 | No | No |
| Responsive | HBCx-14 | WT | 53 | No | Yes |
| | T168 | 1 | 54 | Partial | No |
| Partial | HBCx-11 | 1 | 73 | Partial | Yes |
| Response | | | | | |
| PARP | HBCx-23 | WT | NA | No | No |

### Example 3: Anti-Tumor Activity of Formula I Free Base in the MDA-MB-436 BRCA1 Mutant Human Breast Tumor Model

Anti-tumor activity of the USP1 inhibitor of Formula I free base in comparison to Olaparib and Niraparib was evaluated in mice using the MDA-MB-436 subcutaneous human breast tumor model. 7-9 week old female NOD SCID mice from Beijing Anikeeper Biotech Co. Ltd were injected subcutaneously with 10×10⁶ MDA-MB-436 tumor cells. When tumors reached a volume of approximately 200 mm³ mice were randomized into groups of 10 and dosed via oral gavage with either control, Niraparib (50 mg/kg), Olaparib (75 mg/kg) or the USP1 inhibitor of Formula I at either 30, 100 or 300 mg/kg once daily or 30 mg/kg BID twice daily for 28 days. Body weight and tumor volume was measured twice per week. Tumor volume was calculated as mean and standard error of the mean for each treatment group. The percentage tumor growth inhibition (TGI) was calculated using the mean tumor volume from the treatment group on day 28-day 0/mean tumor volume from control treated group on day 28-day 0 where day 0 is the first day of treatment.

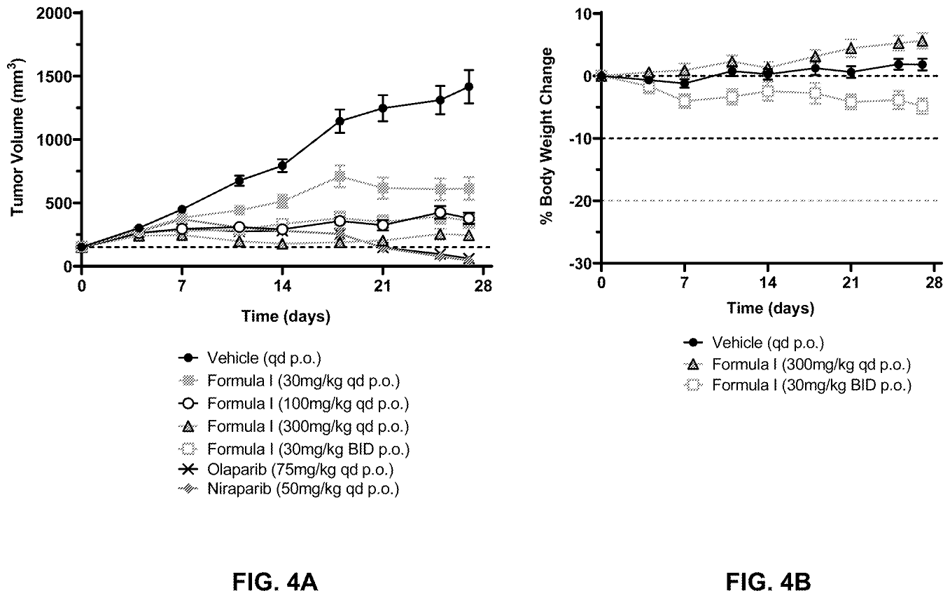

As shown in FIG. 4A, >90% tumor growth inhibition was observed at higher doses qd and BID. As shown in FIG. 4B, doses up to 300 mg/kg were well tolerated in tumor-bearing mice.

### Example 4: Anti-Tumor Activity of Formula I Co-Crystal in the MDA-MB-436 BRCA1 Mutant Human Breast Tumor Model

Anti-tumor activity of the USP1 inhibitor of Formula I co-crystal in comparison to Olaparib and Niraparib was evaluated in mice using the MDA-MB-436 subcutaneous human breast tumor model. 7-9 week old female NOD SCID mice from Beijing Anikeeper Biotech Co. Ltd were injected subcutaneously with 10×10⁶ MDA-MB-436 tumor cells. When tumors reached a volume of approximately 200 mm³ mice were randomized into groups of 10 and dosed via oral gavage once daily for 28 days with either control, Niraparib (50 mg/kg), Olaparib (100 mg/kg) or the USP1 inhibitor of Formula I at either 10, 30, 100 or 300 mg/kg. Body weight and tumor volume was measured twice per week. Tumor volume was calculated as mean and standard error of the mean for each treatment group. The percentage tumor growth inhibition (TGI) was calculated using the mean tumor volume from the treatment group on day 28-day 0/mean tumor volume from control treated group on day 28-day 0 where day 0 is the first day of treatment.

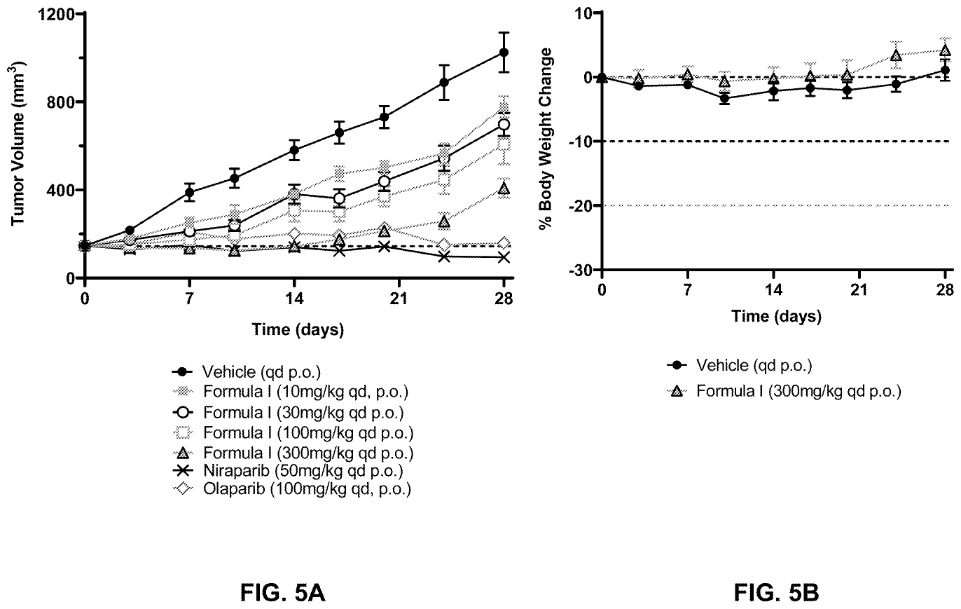

As shown in FIG. 5A, >90% tumor growth inhibition was observed at higher doses qd. Doses up to 300 mg/kg of the Formula I co-crystal were well-tolerated in tumor bearing mice, as shown in FIG. 5B.

### Example 5: Anti-Tumor Activity of Formula I Co-Crystal in Combination with the PARP Inhibitor, Olaparib, in the MDA-MB-436 Human Breast Tumor Mouse Xenograft Model

Anti-tumor activity of the USP1 inhibitor of Formula I co-crystal in combination with Olaparib was evaluated in mice using the MDA-MB-436 subcutaneous human breast tumor model. 7-9 week old female NOD SCID mice from Beijing Anikeeper Biotech Co. Ltd were injected subcutaneously with 10×10⁶ MDA-MB-436 tumor cells. When tumors reached a volume of approximately 200 mm³, mice were randomized into groups of 10 for control, Formula I (100 mg/kg) alone and Formula I (30 mg/kg) alone; or 5 mice for Olaparib (50 mg/kg) alone, Formula I (100 mg/kg) and Olaparib (50 mg/kg) combination group and Formula I (30 mg/kg) and Olaparib (50 mg/kg) combination group. Mice were dosed the relevant treatment via oral gavage once daily for 28 days.

Body weight and tumor volume was measured twice per week. Tumor volume was calculated as mean and standard error of the mean for each treatment group. The percentage tumor growth inhibition (TGI) was calculated using the mean tumor volume from the treatment group on day 28-day 0/mean tumor volume from control treated group on day 28-day 0 where day 0 is the first day of treatment.

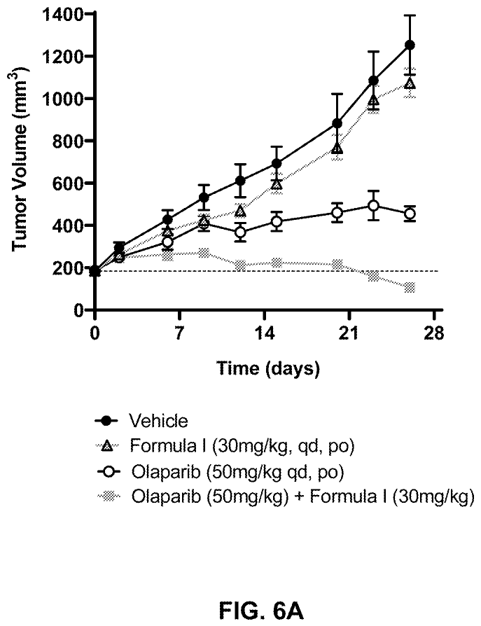

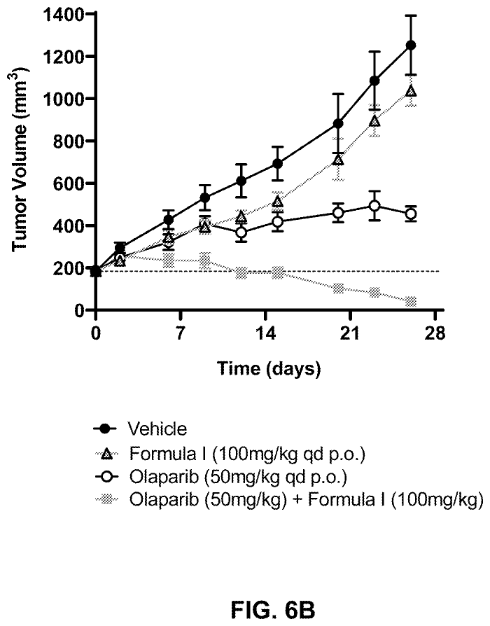

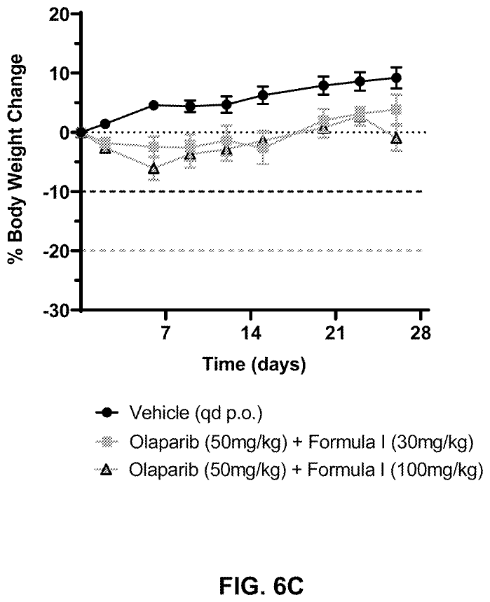

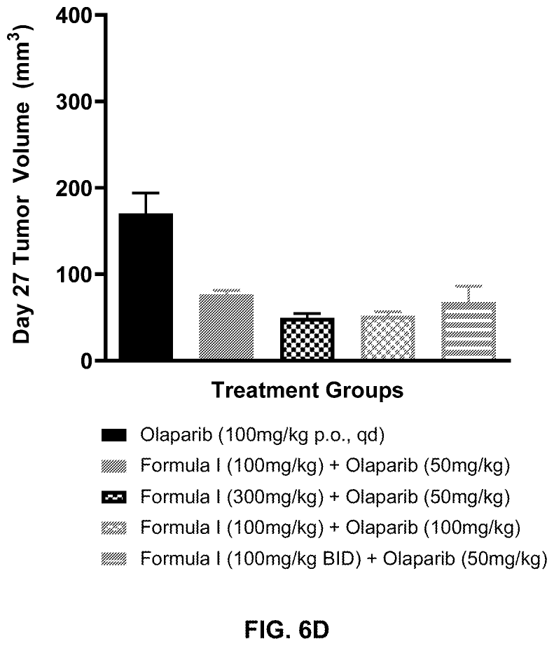

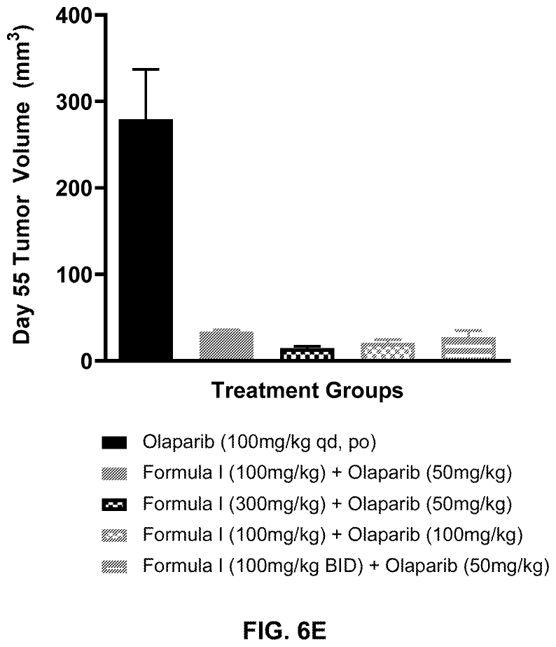

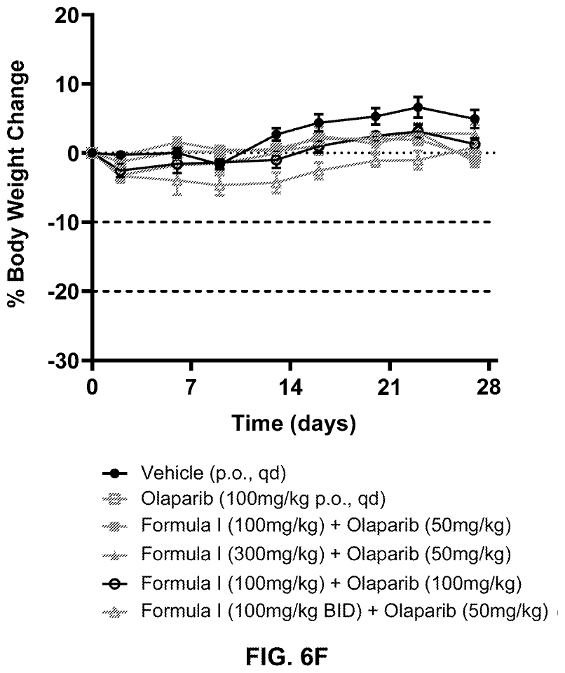

The data in FIGS. 6A and 6B show that, compared to equivalent doses of the single agent of Formula I or Olaparib, the combination treatment groups had enhanced anti-tumor activity in the MDA-MB-436 subcutaneous mouse model. For the Olaparib (50 mg/kg) and Formula I (100 mg/kg) combination study, tolerability was assessed by monitoring body weight and calculating body weight changes as % from body weight on day of treatment start (day 0), as shown in FIG. 6C.

Repeat studies assessing the anti-tumor activity of the USP1 inhibitor of Formula I co-crystal in combination with Olaparib in mice using the MDA-MB=436 subcutaneous human breast tumor model. 7-9 week old female NOD SCID mice from Beijing Anikeeper Biotech Co. Ltd were injected subcutaneously with 10×10⁶ MDA-MB-436 tumor cells. When tumors reached a volume of approximately 200 mm³, mice were randomized into groups of 10 and dosed daily (qd) for control, Formula I (100 mg/kg) alone, Formula I (300 mg/kg) alone, Olaparib (50 mg/kg) alone, Olaparib (100 mg/kg) alone or combination groups of Formula I (100 mg/kg) and Olaparib (50 mg/kg), Formula I (100 mg/kg) and Olaparib (100 mg/kg), Formula I (300 mg/kg) and Olaparib (50 mg/kg); or 6 mice dosed twice daily (BID) for Formula I (100 mg/kg BID) alone, Formula I (100 mg/kg BID) and Olaparib (50 mg/kg) combination group. Mice were dosed the relevant treatment via oral gavage either once daily or twice daily (BID) as highlighted above for 28 days.

Body weight and tumor volume was measured twice per week. Tumor volume was calculated as mean and standard error of the mean for each treatment group. The percentage tumor growth inhibition (TGI) was calculated using the mean tumor volume from the treatment group on day 27-day 0/mean tumor volume from control treated group on day 27-day 0 where day 0 is the first day of treatment.

In all groups containing 10 mice, on day 28 of dosing 6 mice per group were euthanized for ex vivo sample analysis. The remaining 4 mice per group were monitored for response post dose termination.

The data in FIGS. 6D and 6E show that, compared to equivalent doses of the single agent of Formula I or Olaparib, the combination treatment groups had enhanced anti-tumor activity in the MDA-MB-436 subcutaneous mouse model. In addition, all combination groups had enhanced anti-tumor activity compare to the highest dose of Olaparib (100 mg/kg). For all combination groups, tolerability was assessed by monitoring body weight and calculating body weight changes as % from body weight on day of treatment start (day 0), as shown in FIG. 6F. All Formula I and Olaparib combinations were well-tolerated, which was surprising since not all Olaparib combination therapies are well-tolerated. See, e.g., Samol, J., et al., Invest. New Drugs, 30:1493-500 (2012) (“Further development of olaparib and topotecan in combination was not explored due to dose-limiting hematological AEs and the resulting sub-therapeutic MTD.”).

### Example 6: Anti-Tumor Activity of Formula I Co-Crystal in Combination with the PARP Inhibitor, Olaparib, in Patient-Derived Breast Xenograft Models in Nude Mice

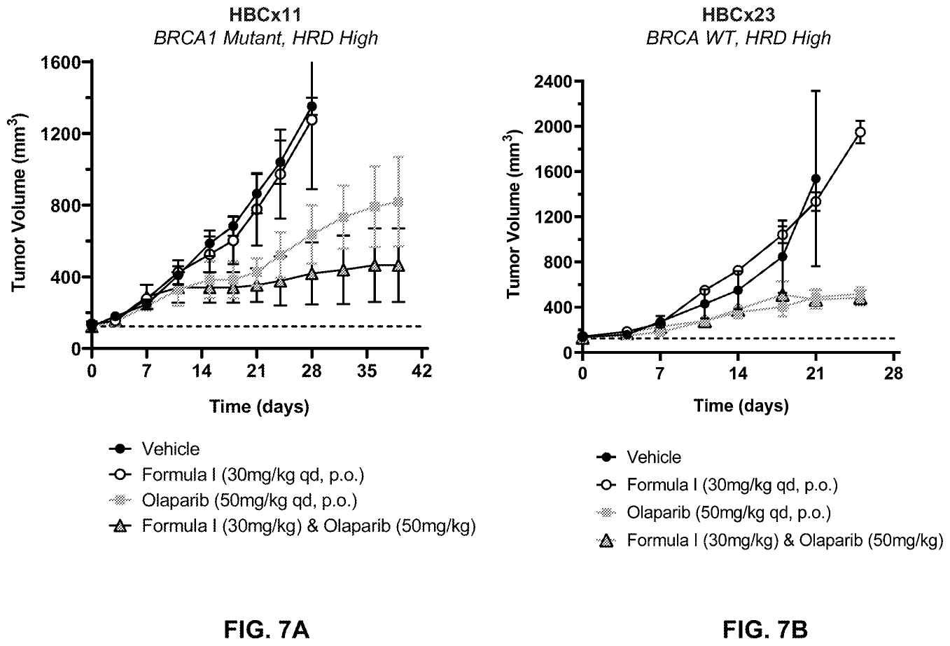

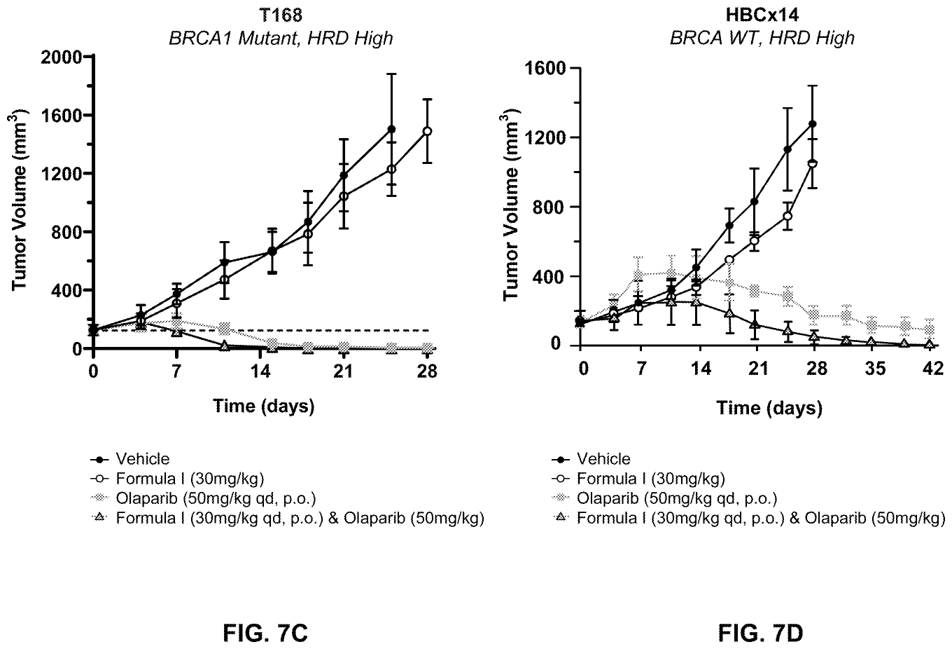

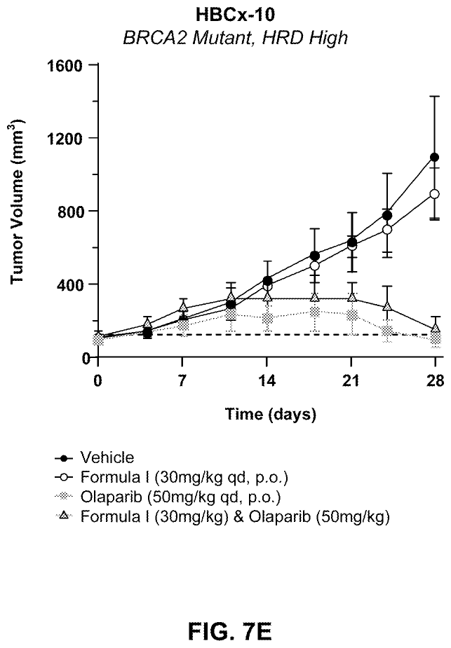

Anti-tumor activity of the USP1 inhibitor of Formula I co-crystal in combination with Olaparib was evaluated in mice using a variety of patient-derived breast xenograft models in nude mice, as shown in FIGS. 7A through 7E. 6-9 week old female Athymic nude mice from Envigo were anesthetized and a 20 mm³ tumor fragment placed subcutaneously via incision in the flank. When tumors established to a tumor volume ranging from 60 to 320 mm³ mice were randomized into groups of 3 and were assigned into the following groups: control, Formula I (30 mg/kg), Olaparib (50 mg/kg) or Formula I (30 mg/kg) and Olaparib (50 mg/kg) combination. Compound was administered via oral gavage once daily for up to 42 days dependent upon the growth kinetics of the tumor model. Body weight and tumor volume were measured twice per week. Tumor volume was calculated as mean and standard error of the mean for each treatment group.

The data in FIG. 7D show that, compared to equivalent doses of the single agent of Formula I or Olaparib, the combination treatment group showed enhanced anti-tumor activity in the HBCx-14 patient-derived subcutaneous mouse model. The data in FIG. 7A show potential combination advantages in the HBCx11 patient-derived subcutaneous mouse model.

### Example 7: Anti-Tumor Activity of Formula I Co-Crystal in Combination with the PARP Inhibitor, Olaparib, in the HBCx-11 BRCA1 Mutant HRD High Human Breast PDX Model

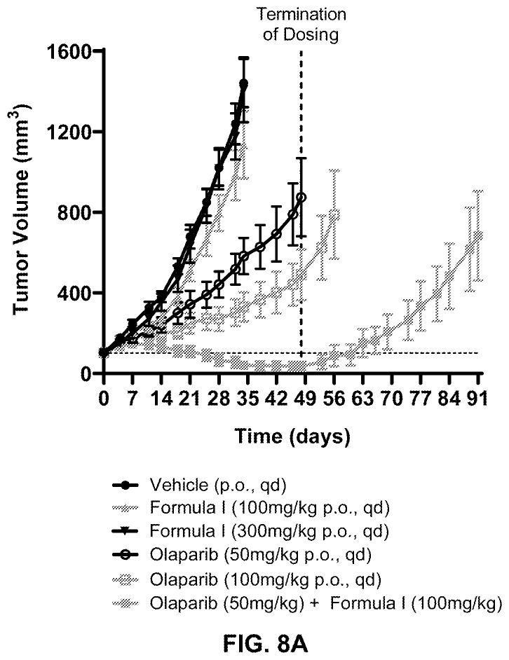

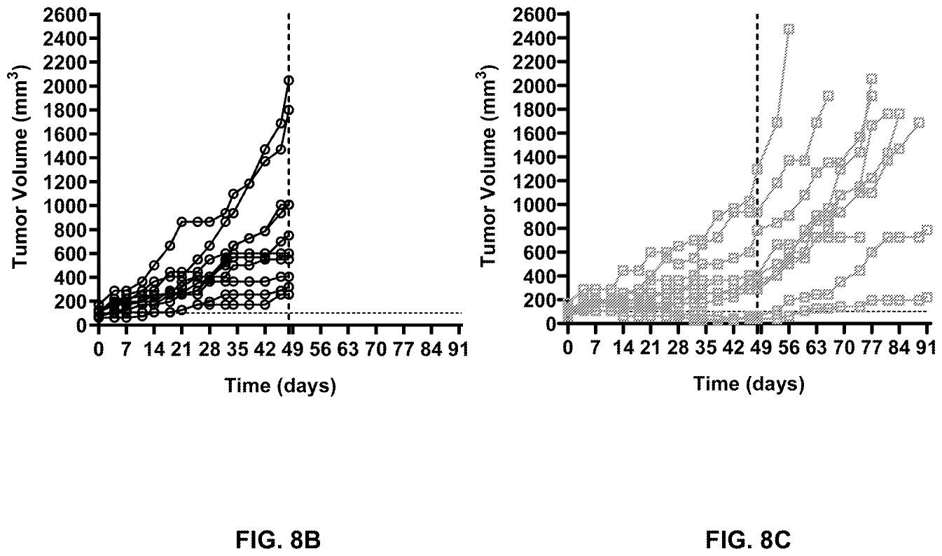

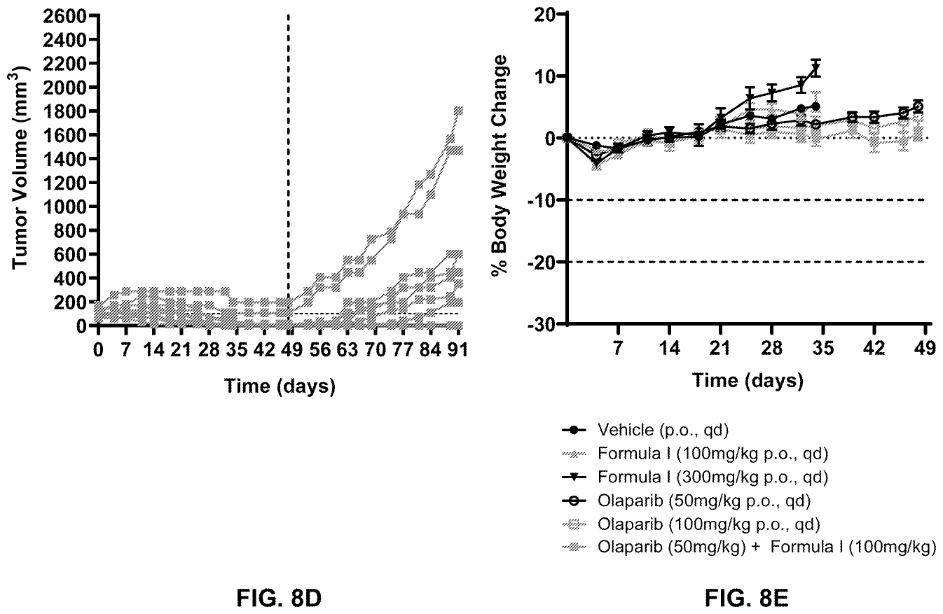

Anti-tumor activity of the USP1 inhibitor of Formula I co-crystal in combination with Olaparib was evaluated in the HBCx-11 BRCA1 mutant HRD high human breast PDX model, as shown in FIGS. 8A-8E. The HBCx-11 model is RAD51 high model, and HRD high refers to the Myriad HRD biomarker (defined as deleterious or suspected deleterious mutations in BRCA1 and BRCA2 genes and/or positive Genomic Instability Score (GIS); GIS is an algorithmic measurement of Loss of Heterozygosity (LOH), Telomeric Allelic Imbalance (TAI), and Large-scale State Transitions (LST) using DNA isolated from formalin-fixed paraffin embedded (FFPE) tumor tissue specimens; see myriad.com/products-services/precision-medicine/mychoice-cdx/). 6-9 week old female Athymic nude mice from Envigo were anesthetized and a 20 mm³ tumor fragment was placed subcutaneously via incision in the flank. When tumors established to a tumor volume ranging from 60 to 200 mm³ mice were randomized into groups of 10 and were assigned into the following groups: control; Formula I (300 mg/kg), Formula I (100 mg/kg), Olaparib (50 mg/kg), Olaparib (100 mg/kg) or Formula I (100 mg/kg) and Olaparib (50 mg/kg) combination. Compound was administered via oral gavage once daily for up to 49 days (day 0 to day 48). Body weight and tumor volume were measured twice per week. Tumor volume was calculated as mean and standard error of the mean for each treatment group.

The data in FIGS. 8A-8D show that, compared to equivalent doses of the single agent of Formula I or Olaparib, the combination treatment group showed enhanced anti-tumor activity in the HBCx-11 BRCA1 mutant HRD high human breast PDX model. In addition, the combination treatment had enhanced anti-tumor activity compared to the highest dose of Olaparib (100 mg/kg). The body-weight data in FIG. 8E show that the combination was well-tolerated.

### Example 8: Anti-Tumor Activity of Formula I Co-Crystal in Combination with the PARP Inhibitor, Olaparib, in the HBCx-14 Patient-Derived Breast Xenograft Model in Nude Mice

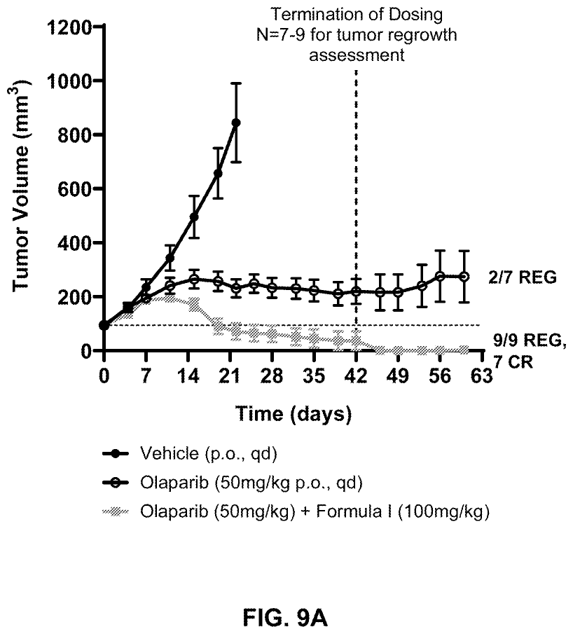

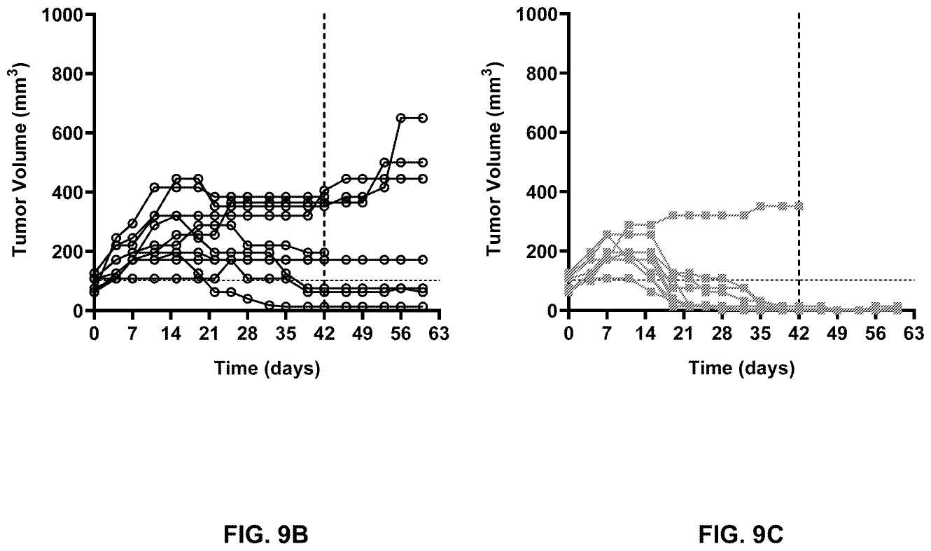

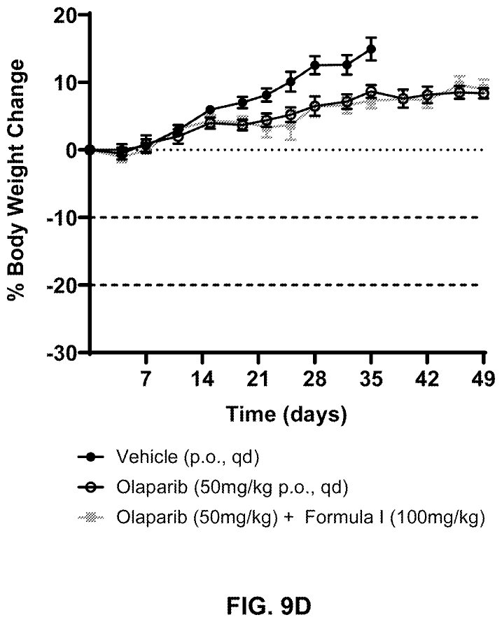

Anti-tumor activity of the USP1 inhibitor, Formula I co-crystal in combination with Olaparib was evaluated in mice using a variety of patient-derived breast xenograft models in nude mice, as shown in FIGS. 9A-9D. 6-9 week old female Athymic nude mice from Envigo were anesthetized and a 20 mm³ tumor fragment placed subcutaneously via incision in the flank. When tumors established to a tumor volume ranging from approximately 60 to 130 mm³ mice were randomized into groups of 10 and were assigned into the following groups: control, Olaparib (50 mg/kg), or Formula I (100 mg/kg) and Olaparib (50 mg/kg) combination. Compound was administered via oral gavage once daily for 42 days (day 1 to 42). Body weight and tumor volume were measured twice per week. Tumor volume was calculated as mean and standard error of the mean for each treatment group. A tumor regression (REG) was defined as a tumor with a smaller volume of the last day of the study as compared to the first day of dosing, and a complete regression (CR) was defined as no palpable tumor at the end of the study.

The data in FIG. 9A-9C show that, compared to equivalent doses of single agent Olaparib, the combination treatment group showed enhanced anti-tumor activity in the HBCx-14 patient-derived subcutaneous mouse model. The body-weight data in FIG. 9D show that the combination was well-tolerated.

### Example 9. Anti-Tumor Activity of Formula I Co-Crystal in Combination with the PARP Inhibitor, Olaparib, in the OV0589 Patient-Derived Ovarian Xenograft Model in Nude Mice

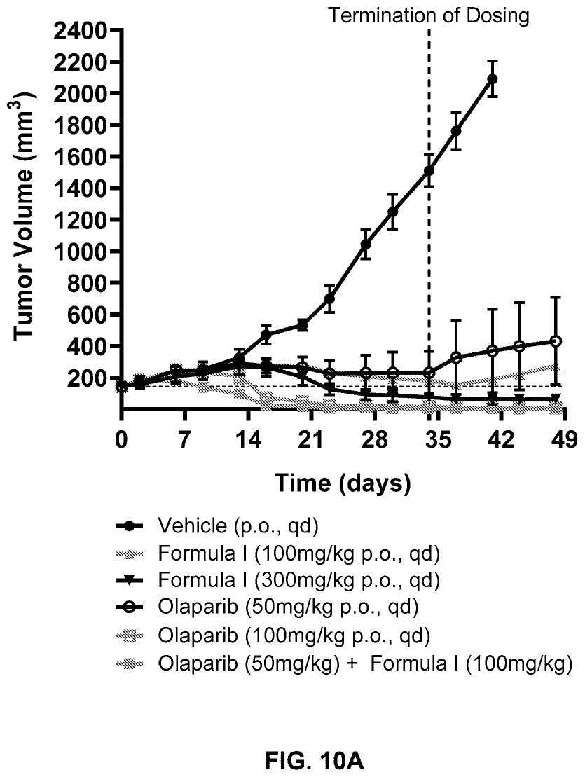

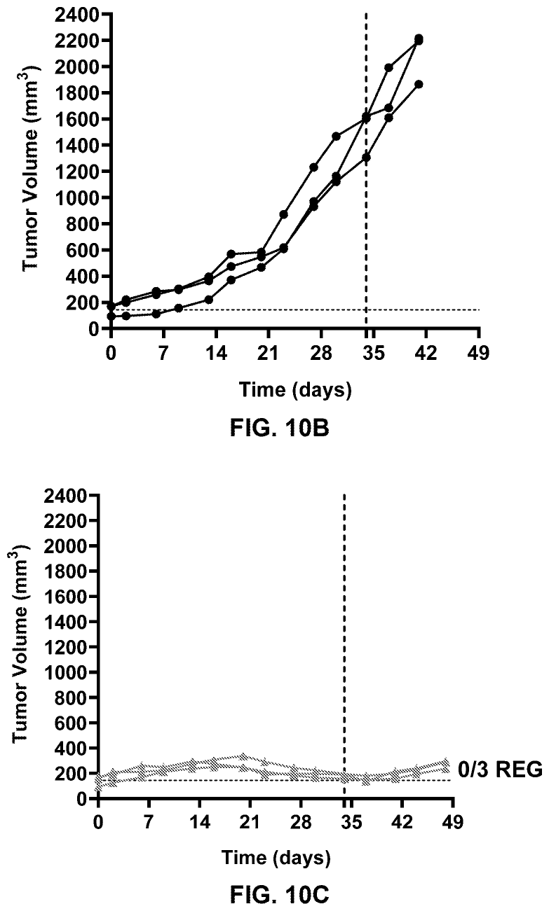

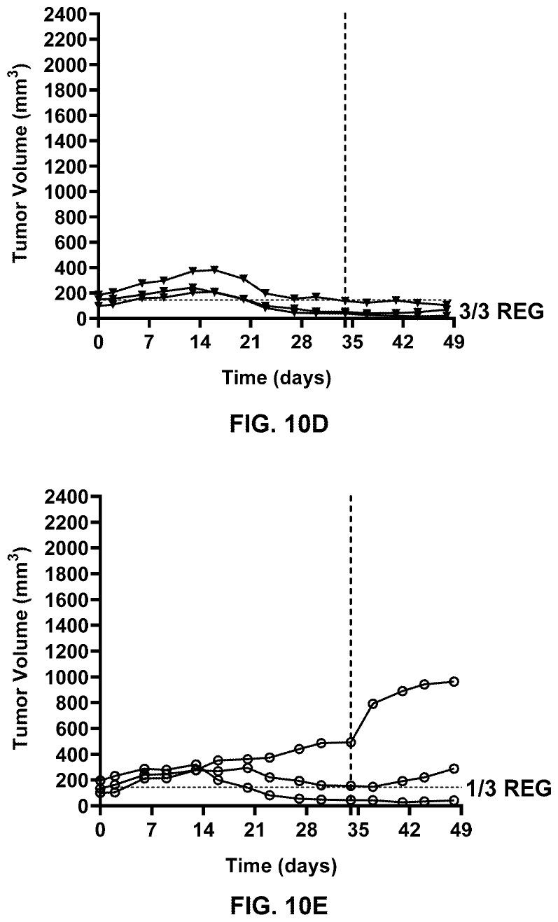

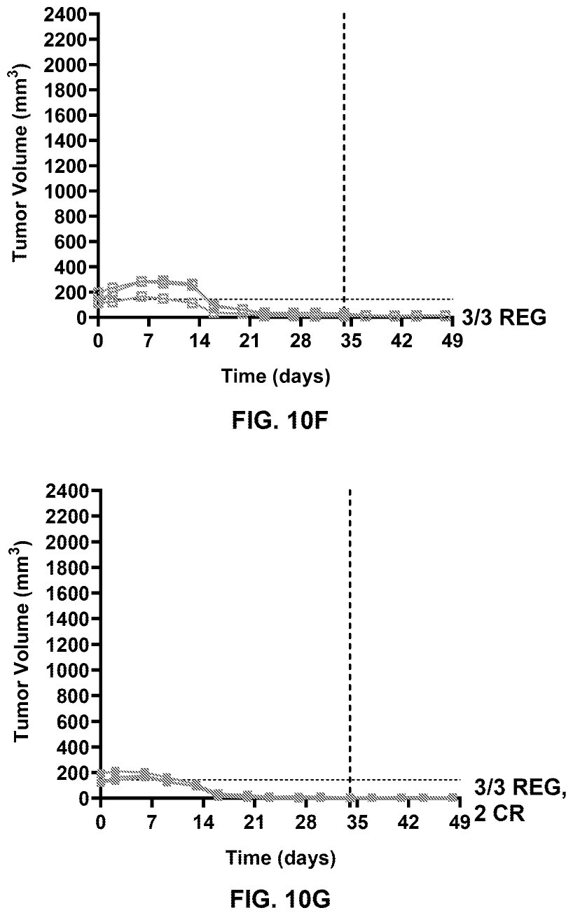

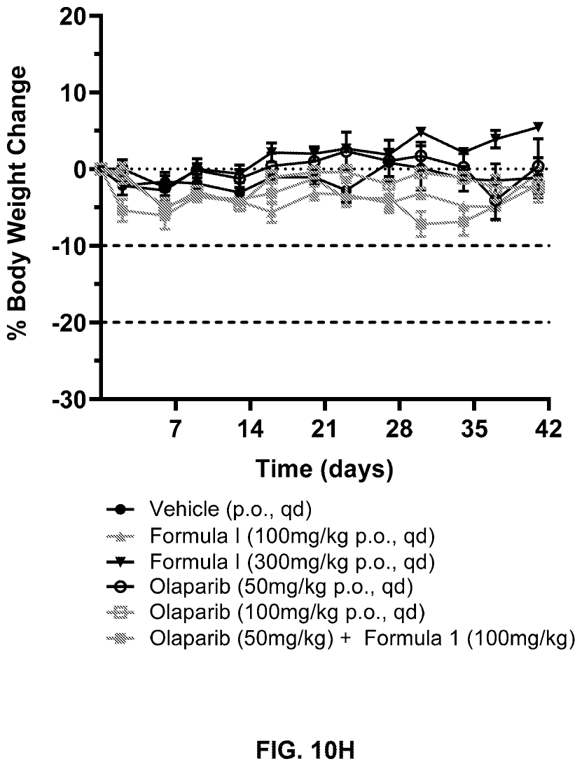

Anti-tumor activity of the USP1 inhibitor, Formula I co-crystal in combination with Olaparib was evaluated in mice using three patient-derived ovarian xenograft models in nude mice, as shown in FIGS. 10A-10H. 6-8 week old female BALB/c nude mice from Beijing Anikeeper Biotech Co., Ltd were anesthetized and a 2-3 mm in diameter tumor fragment placed subcutaneously via incision in the flank. When tumors established to a tumor volume ranging from approximately 90 to 200 mm³ mice were randomized into groups of 3 and were assigned into the following groups: control, Formula I (100 mg/kg), Formula I (300 mg/kg), Olaparib (50 mg/kg), Olaparib (100 mg/kg) or Formula I (100 mg/kg) and Olaparib (50 mg/kg) combination. Compound was administered via oral gavage once daily for 35 days (day 0 to 34). Body weight and tumor volume were measured twice per week. Tumor volume was calculated as mean and standard error of the mean for each treatment group. A tumor regression (REG) was defined as a tumor with a smaller volume on the last day of the study as compared to the first day of dosing, and a complete regression (CR) was defined as no palpable tumor at the end of the study.

The data in FIGS. 10A-10G show that, compared to equivalent doses of the single agent of Formula I or Olaparib, the combination treatment group showed enhanced anti-tumor activity in the OV0589 patient-derived ovarian subcutaneous mouse model. In addition, the combination treatment was as efficacious the highest dose of Olaparib (100 mg/kg). Formula I treatment also showed anti-tumor activity alone at both doses. Body weight measurements indicate that all treatments were well tolerated (FIG. 10H).

### Example 10. Anti-Tumor Activity of Formula I Co-Crystal in Combination with the PARP Inhibitor, Olaparib, in the ST416 Patient-Derived Ovarian Xenograft Model in Nude Mice

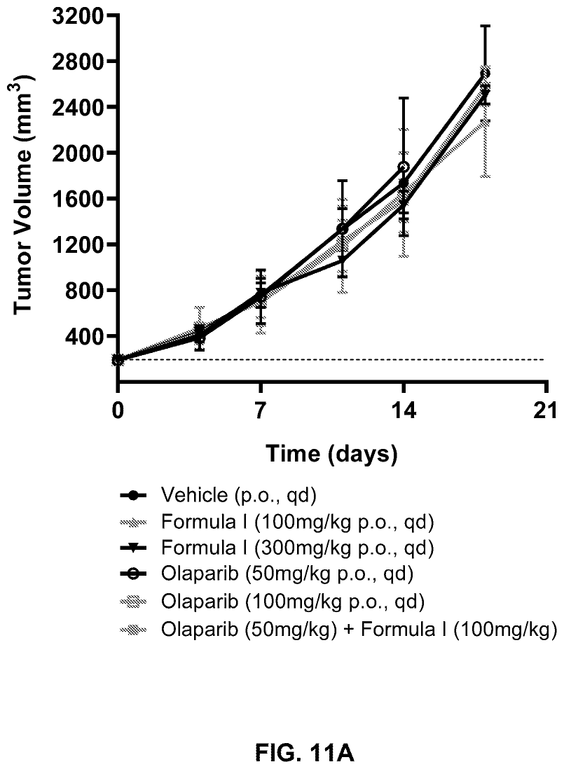

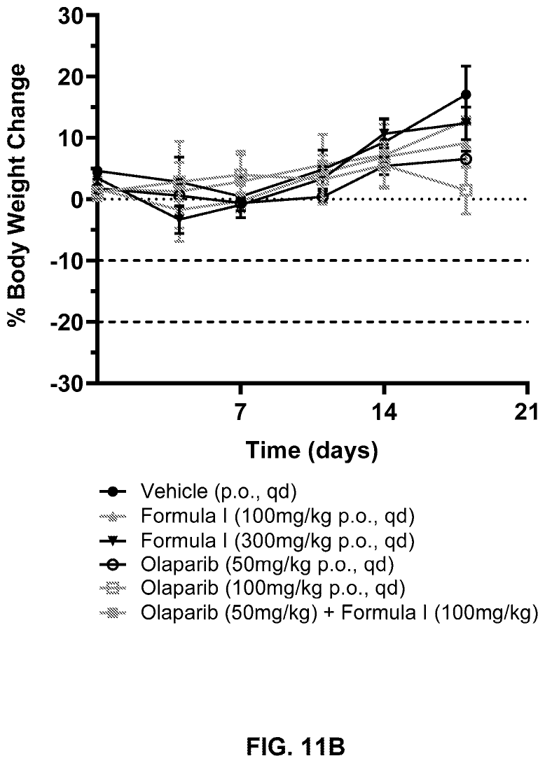

Anti-tumor activity of the USP1 inhibitor, Formula I co-crystal in combination with Olaparib was evaluated in mice using three patient-derived ovarian xenograft models in nude mice, as shown in FIGS. 11A and 11B. 6-8 week old female athymic nude mice from The Jackson Laboratory were anesthetized and a 70 mm³ tumor fragment placed subcutaneously via incision in the flank. When tumors established to a tumor volume ranging from approximately 65 to 130 mm³ mice were randomized into groups of 3 and were assigned into the following groups: control, Formula I (100 mg/kg), Formula I (300 mg/kg), Olaparib (50 mg/kg), Olaparib (100 mg/kg) or Formula I (100 mg/kg) and Olaparib (50 mg/kg) combination. Compound was administered via oral gavage once daily for 19 days (day 0 to 18). Body weight and tumor volume were measured twice per week. Tumor volume was calculated as mean and standard error of the mean for each treatment group. A tumor regression (REG) was defined as a tumor with a smaller volume on the last day of the study as compared to the first day of dosing, and a complete regression (CR) was defined as no palpable tumor at the end of the study.

The data in FIG. 11A shows no anti-tumor activity in any treatment groups in the ST416 patient-derived ovarian subcutaneous mouse model. Body weight measurements indicate that all treatments were well tolerated (FIG. 11B).

### Example 11. Anti-Tumor Activity of Formula I Co-Crystal in Combination with the PARP Inhibitor, Olaparib, in the CTG-0253 Patient-Derived Ovarian Xenograft Model in Nude Mice

Anti-tumor activity of the USP1 inhibitor, Formula I co-crystal in combination with Olaparib is evaluated in mice using three patient-derived ovarian xenograft models in nude mice. 6-8 week old female athymic nude mice from Engivo are anesthetized and a 125 mm³ tumor fragment is placed subcutaneously via incision in the flank. When tumors establish to a tumor volume ranging from approximately 130 to 240 mm³ mice are randomized into groups of 3 and were assigned into the following groups: control, Formula I (100 mg/kg), Formula I (300 mg/kg), Olaparib (50 mg/kg), Olaparib (100 mg/kg) or Formula I (100 mg/kg) and Olaparib (50 mg/kg) combination. Compound is administered via oral gavage once daily for 20 days. Body weight and tumor volume are measured twice per week. Tumor volume is calculated as mean and standard error of the mean for each treatment group. A tumor regression (REG) is defined as a tumor with a smaller volume on the last day of the study as compared to the first day of dosing, and a complete regression (CR) is defined as no palpable tumor at the end of the study.

### Example 12. Anti-Tumor Activity of Formula I Co-Crystal in Combination with the PARP Inhibitor, Olaparib, in the CTG-0253 Patient-Derived Ovarian Xenograft Model in Nude Mice

Anti-tumor activity of the USP1 inhibitor, Formula I co-crystal in combination with Olaparib was evaluated in mice using three patient-derived ovarian xenograft models in nude mice. 6-8 week old female athymic nude mice from Engivo were anesthetized and a 60 mm³ tumor fragment was placed subcutaneously via incision in the flank. When tumors reached a tumor volume ranging from approximately 100 to 180 mm³, mice were randomized into groups of 3-4 mice and were assigned into the following groups: control (vehicle), Formula I (100 mg/kg), Formula I (300 mg/kg), Olaparib (100 mg/kg) or Formula I (100 mg/kg) and Olaparib (100 mg/kg) combination. Compound was administered via oral gavage once daily for 18 days (days 0 to 17). Body weight and tumor volume were measured twice per week. Tolerability was assessed by calculating body weight changes as percent (%) from body weight on day of treatment start (day 0). Tumor volume was calculated as mean and standard error of the mean for each treatment group. A tumor regression (REG) was defined as a tumor with a smaller volume on the last day of the study as compared to the first day of dosing, and a complete regression (CR) was defined as no palpable tumor at the end of the study.

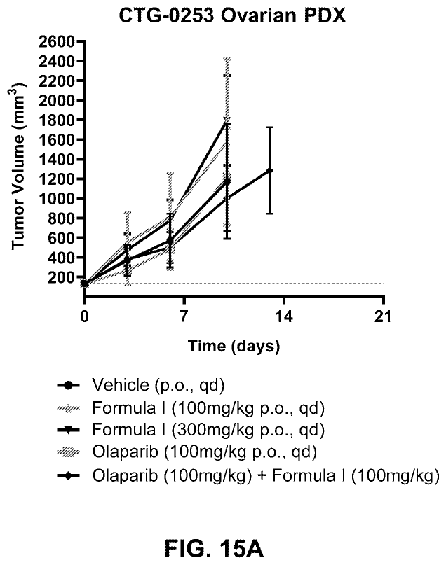

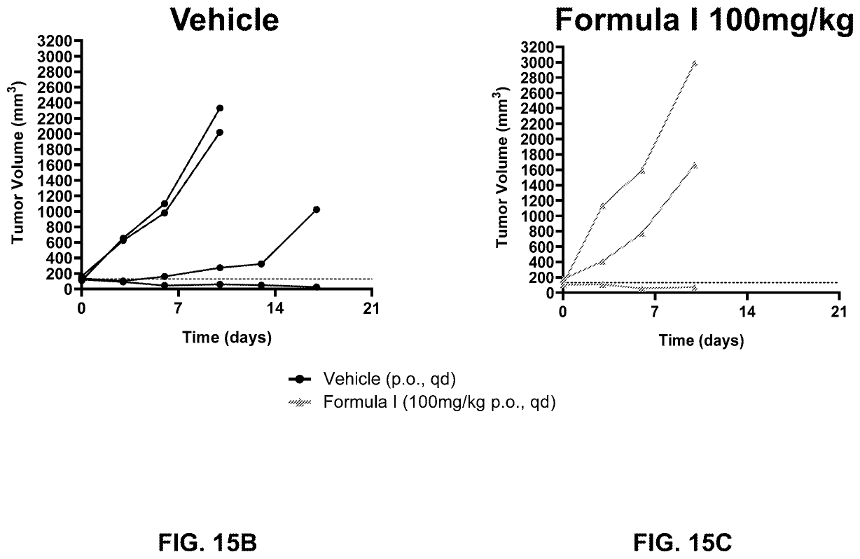

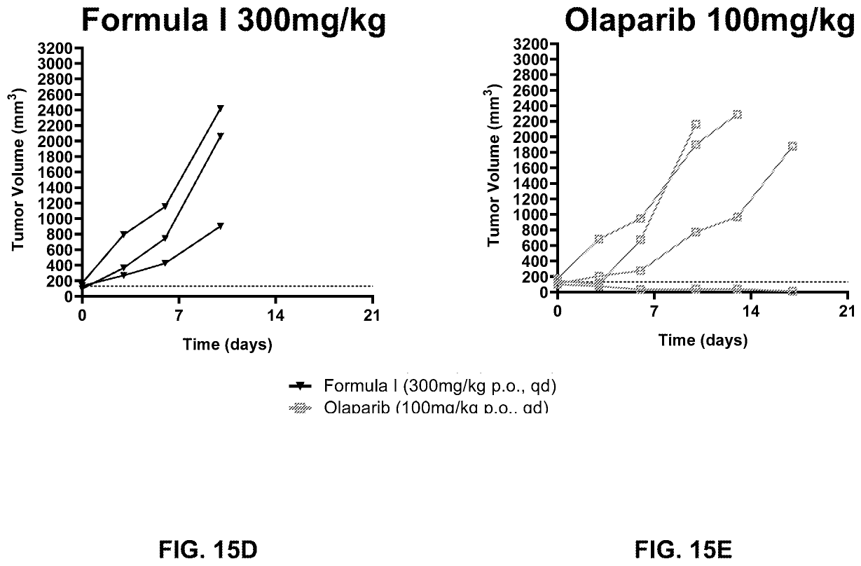

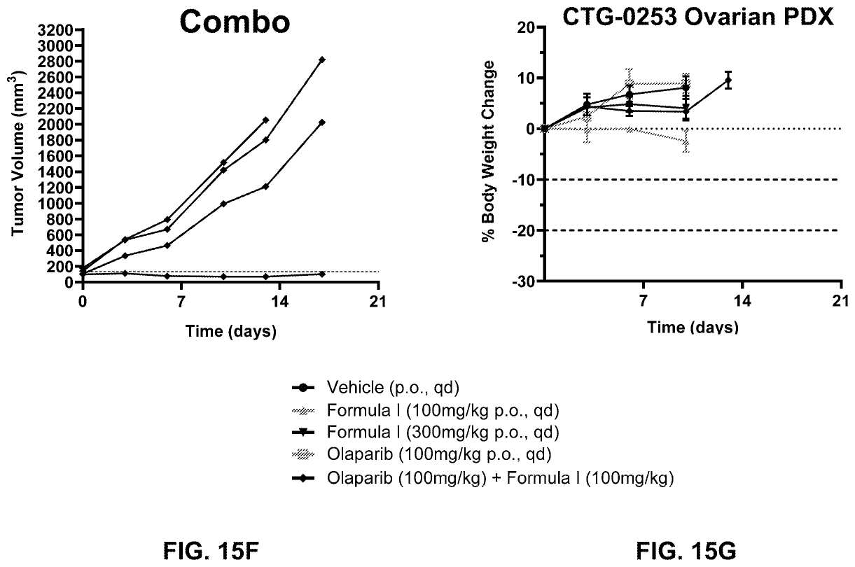

The data in FIGS. 15A-F shows no anti-tumor activity in any treatment groups in the CTG-0253 patient-derived ovarian xenograft mouse model. The body weight data in FIG. 15G show that the combination was well tolerated.

### Example 13. Anti-Tumor Activity of Formula I Co-Crystal in Combination with the PARP Inhibitor, Niraparib, in the MDA-MB-436 Human Breast Tumor Mouse Xenograft Model

Anti-tumor activity of the USP1 inhibitor, Formula I co-crystal in combination with Niraparib was evaluated in mice using the MDA-MB-436 subcutaneous human breast tumor model. 7-9 week old female NOD SCID mice from Beijing Anikeeper Biotech Co. Ltd were injected subcutaneously with 10×106 MDA-MB-436 tumor cells. When tumors reached a volume of approximately 319 mm³, mice were randomized into groups of 5 and dosed daily (qd) for control, Niraparib (20 mg/kg) alone, Niraparib (50 mg/kg) alone or a combination group of Formula I (100 mg/kg) and Niraparib (20 mg/kg). Mice were dosed the relevant treatment via oral gavage once daily as highlighted above for 28 days.

Body weight and tumor volume was measured at least twice per week. Tumor volume was calculated as mean and standard error of the mean for each treatment group.

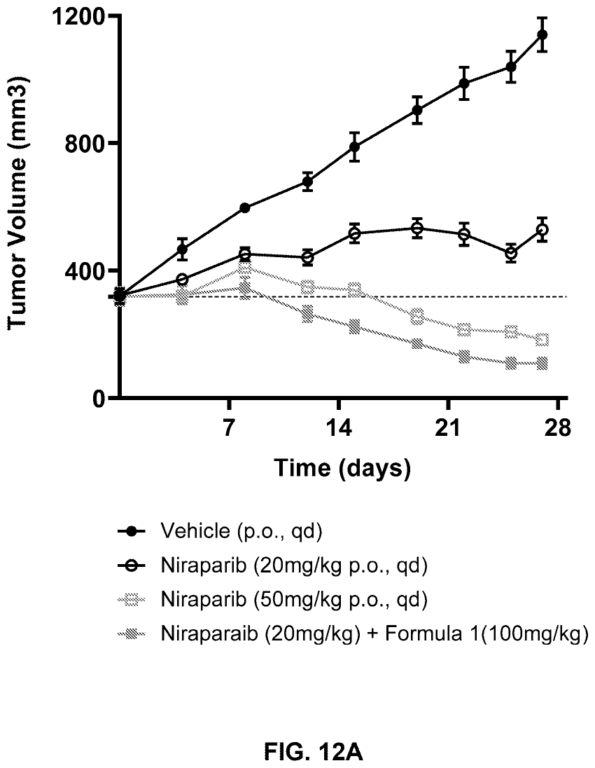

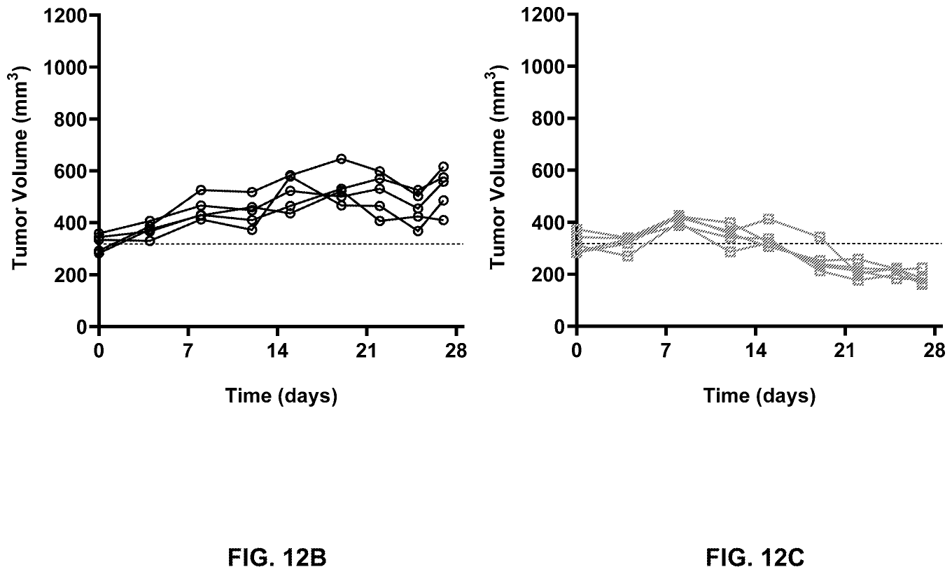

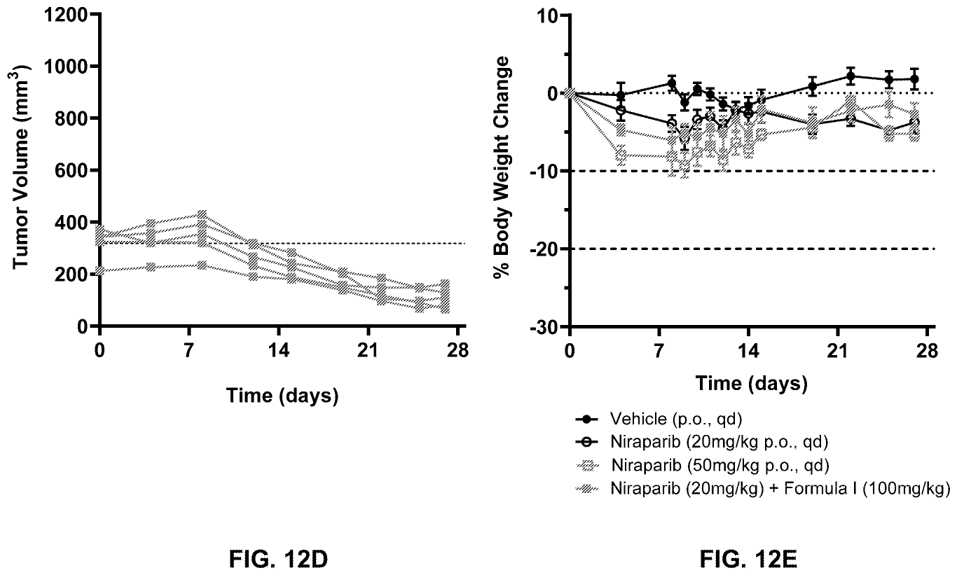

The data in FIGS. 12A-12C show that, compared to equivalent doses of the single agent of Niraparib, the combination treatment group had enhanced anti-tumor activity in the MDA-MB-436 subcutaneous mouse model. In addition, the combination group had enhanced anti-tumor activity compared to the highest dose of Niraparib (50 mg/kg). For the combination group, tolerability was assessed by monitoring body weight and calculating body weight changes as % from body weight on day of treatment start (day 0), as shown in FIG. 12D. Body weight measurements indicate that the combination treatment was well tolerated (FIG. 12E).

### Example 14. DDI of Formula I Co-Crystal in Combination with the PARP Inhibitor, Olaparib, in Non-Tumor Bearing NOD SCID Female Mice

The drug-drug interaction (DDI) of the USP1 inhibitor Formula I co-crystal in combination with Olaparib was evaluated in mice by assessing plasma systemic exposure over time. 6-8 week old female NOD SCID mice from Beijing Anikeeper Biotech Co. Ltd were randomized into groups of 4 and dosed via oral gavage once daily for 5 days with Formula I (100 mg/kg) alone, Olaparib (50 mg/kg) alone, or with Formula I (100 mg/kg) and Olaparib (50 mg/kg) in combination.

For Olaparib (50 mg/kg) alone or in combination with for Formula I (100 mg/kg), blood samples were collected from each mouse at the following time points post day 1 and day 5 dose: pre-dose, 0.5 hr, 1 hr, 2 hr, 6 hr, 12 hr, 24 hr. For Formula I alone (100 mg/kg) or in combination with Olaparib (50 mg/kg), blood samples were collected from each mouse at the following time points post day 1 and day 5: pre-dose, 1 hr, 2 hr, 4 hr, 8 hr, 12 hr, and 24 hr.

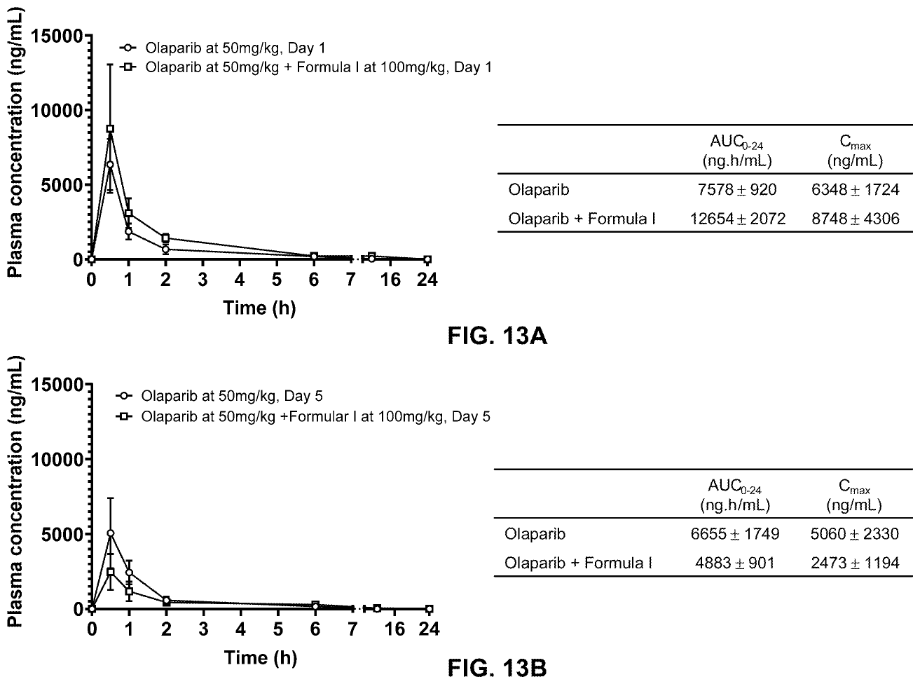

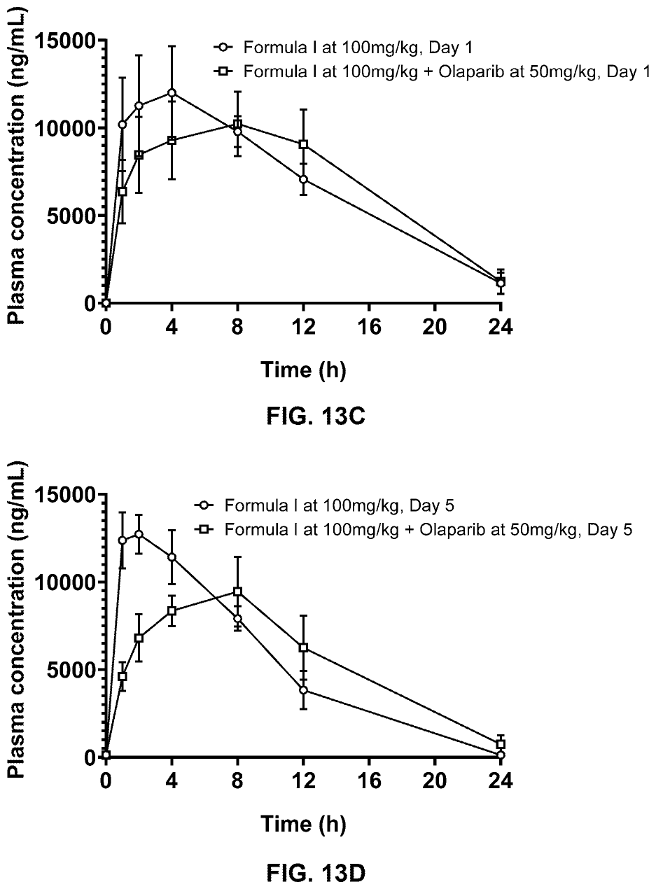

The data in FIGS. 13A-13D show that co-administration of Formula I and Olaparib does not increase Formula I exposure (13A and 13B) nor does it increase Olaparib exposure (13C and 13D). Thus, the combination activity is not due to an increase of Formula I exposure or Olaparib exposure.

### Example 15. Ten-Day Exploratory Toxicity Study of Formula I Co-Crystal in Sprague-Dawley Rats and Cynomolgus Monkeys

### A. Ten-Day Oral Dose Exploratory Toxicity Study for Formula I Co-Crystal in Sprague-Dawley Rats

In order to evaluate the toxicity and toxicokinetics of Formula I co-crystal, Formula I co-crystal was administered to male Sprague-Dawley rats for 10 days via oral gavage. Twenty-five 7-8 week old male rats (Rattus norvegicus) (5 mice/group) from (Envigo RMS, Inc., Indianapolis, Ind.) were administered vehicle or test article for ten days as either daily (SID) or twice daily (BID) oral doses as described in Table 4. Whole venous blood samples of approximately 0.5 mL were collected from a peripheral vein of the rats for determination of test article exposure. Samples were collected no Days 1 and 10: prior to administration (Day 10 only) and at 30 minutes, 1 hr, 2 hr, 4 hr, 8 hr, and 24 hrs after test article administration. All animals were euthanized for postmortem examinations approximately twenty-four hours post last dose.

Toxicokinetic analyses were conducted using Phoenix WinNonlin software (Version 8.1 or higher) using non-compartmental approach based on the route of administration.

TABLE 4
Dosage schedule for toxicology study of Formula
I co-crystal in Sprague-Dawley rats

| | | Total | Total | | | | |
|---|---|---|---|---|---|---|---|
| | | Daily | Daily | | | Number | |
| | Test | Dosage | Dosage | Concentration | Dosing | of | Animal |
| Group | Article | (mg/kg) | (mg/kg) | (mg/mL)** | Regimen | Animals | No. (M) |
| 1 | Vehicle* | 0 (vehicle) | — | 0 | SID | 5 | 1-5 |
| 2 | Formula I | 100 | — | 10 | SID | 5 | 6-10 |
| | co-crystal | | | | | | |
| 3 | Formula I | 300 | — | 30 | SID | 5 | 11-15 |
| | co-crystal | | | | | | |
| 4 | Formula I | 1000 | — | 100 | SID | 5 | 16-20 |
| | co-crystal | | | | | | |
| 5 | Formula I | 300 | 150 | 15 | BID*** | 5 | 21-25 |
| | co-crystal | | | | | | |

*0.5% HPMC/0.1% Tween-80
**A correction factor of 1.288 was required to correct for the presence of gentisic acid
***dosing was approximately 12 hours apart

### B. Ten-Day Exploratory Toxicity Study of Formula I Co-Crystal in Cynomolgus Monkeys

To evaluate the toxicity and toxicokinetics of Formula I co-crystal, Formula I co-crystal was administered daily to male cynomolgus monkeys for 10 days. Fifteen 2-3 year old male cynomolgus monkeys (Macaca fascicularis) (3 animals/group) from Orient BioResource (Alice, Tex.) were administered vehicle or test articles via oral gavage for ten days as described in Table 5. In life, animals were observed for clinical signs of toxicity, changes in body weight and food consumption. Serial blood samples were collected for plasma concentration analysis to evaluate systemic test article exposure. All animals were euthanized for postmortem examinations approximately twenty-four hours post last dose (12 hours post last dose for BID arm).

Toxicokinetic analyses were conducted using Phoenix WinNonlin software (Version 8.1 or higher) using non-compartmental approach based on the route of administration.

TABLE 5
Dosage schedule for toxicology study of Formula
I co-crystal in cynomolgus monkeys

| | Total | Total | | | |
|---|---|---|---|---|---|
| | Daily | Daily | Concen- | Number | Animal |
| | Dosage | Dosage | tration | of | No. |
| Group | (mg/kg) | (mg/kg) | (mg/mL) | Animals | (M) |
| 1 | 0 (vehicle)* | — | 0 | 3 | 1-3 |
| 2 | 100 | — | 20 | 3 | 4-6 |
| 3 | 300 | — | 60 | 3 | 7-9 |
| 4 | 1000 | — | 200 | 3 | 10-12 |
| 5 | 300 | 150 BID *** | 30 | 3 | 12-15 |

*0.5% HPMC/0.1% Tween-80
** A correction factor of 1.288 is required for the presence of gentisic acid
*** Dosing occurred approximately 12 hours apart

### C. Toxicity Results of Formula I Co-Crystal in Sprague-Dawley Rats and Cynomolgus Monkeys

A summary of the toxicity and toxicokinetic studies of Formula I co-crystal in Sprague-Dawley rats and cynomolgus monkeys in comparison to various PARP inhibitors is shown in Table 6. In comparison to various PARP inhibitors for which dose limiting toxicity is hematopoietic toxicity (myelosuppression and pancytopenia), the dose limiting toxicity for Formula I co-crystal is GI toxicity and thus, has non-overlapping dose limiting toxicity with PARP inhibitors. Table 6 further shows that hematopoietic toxicity is dose limiting for all approved PARP inhibitors. The clinical dose interruption, reduction, and discontinuation of PARP inhibitors are common due to patients experiencing adverse events when administered PARP inhibitors, so there is a need for a more tolerable and efficacious PARP inhibitor combination regimen. Thus, an opportunity exists for combination USP1 inhibitor and PARP inhibitor treatment, which could further enhance the efficacy of PARP inhibitors at reduced dose without overlapping toxicity.

TABLE 6
Comparison of dose limiting toxicities between Formula
I co-crystal and various PARP inhibitors

| USP1 inhibitor | Summary of Toxicity Results |
|---|---|
| Formula I co-crystal | Gastrointestinal toxicity was dose limiting in both rats and cynomolgus |
| | monkeys. The gastric changes, which were observed at exposures ≥6x |
| | the predicted human free AUCₛₛ (monotherapy) and ≥13x |
| | (combination) were suggestive of an irritant effect |
| | Hematopoietic toxicity was not a prominent feature in either species |
| | In rats, neither anemia nor pancytopenia was noted at any dose level |
| | In cynomolgus monkeys, decreases in red blood cells and white blood |
| | cells were noted, although these changes were not considered sufficient |
| | to have caused moribundity |
| Olaparib | Olaparib caused marked hematological toxicities in both a 7-day rat |
| (NDA # 206162) | study and a maximum tolerated dose study in dogs |
| Niraparib | Niraparib caused early deaths due to bone marrow toxicity observed in |
| (NDA #208447) | a 90-day rat study |
| | Significant changes were noted in hematology parameters in a 28-day |
| | dog study with niraparib |
| Rucaparib | Decreased red cell mass correlated with atrophy in the bone marrow |
| (NDA # 209115) | was observed when Rucaparib was used in a 28-day rat study |
| Talazoparib | Dose limiting toxicity was due to hematology findings in a 90-day rat |
| (NDA # 211651) | study |

### Example 16: Anti-Tumor Activity of Formula I Co-Crystal in Combination with the PARP Inhibitor, Olaparib, in the Breast HBCx-8 Patient-Derived Breast Xenograft Model in Nude Mice

Anti-tumor activity of the USP1 inhibitor, Formula I co-crystal in combination with Olaparib was evaluated in mice using a variety of patient-derived xenograft models, including the triple negative breast cancer BRCA1 mutant, TP53 mutant, HRD high, and RAD51 high HBCx-8 model. 6-9 week old female athymic nude mice from Envigo were anesthetized, and a 20 mm³ tumor fragment was placed subcutaneously via incision in the flank. When tumors reached a tumor volume ranging from approximately 60 to 130 mm³, mice were randomized into groups of 3 mice and were assigned into the following groups: control (vehicle), Formula I (100 mg/kg), Olaparib (100 mg/kg), or Formula I (100 mg/kg) and Olaparib (100 mg/kg) combination. Compound was administered via oral gavage once daily for 42 days (days 0 to 41). Body weight and tumor volume were measured twice per week. Tolerability was assessed by calculating body weight changes as percent (%) from body weight on day of treatment start (day 0). Tumor volume was calculated as mean and standard error of the mean for each treatment group. A tumor regression (REG) was defined as a tumor with a smaller volume of the last day of the study as compared to the first day of dosing, and a complete regression (CR) was defined as no palpable tumor at the end of the study.

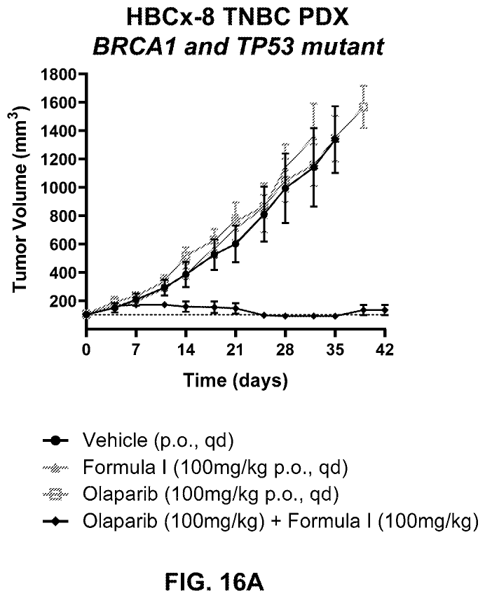

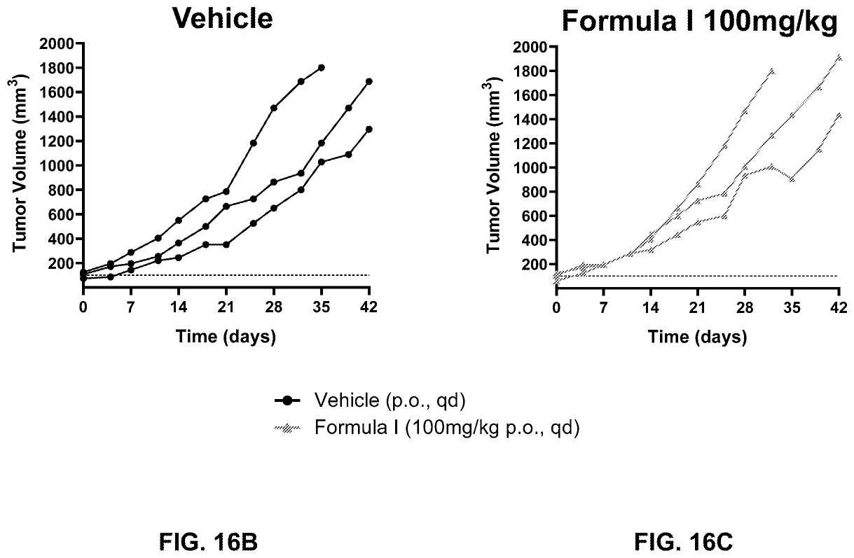

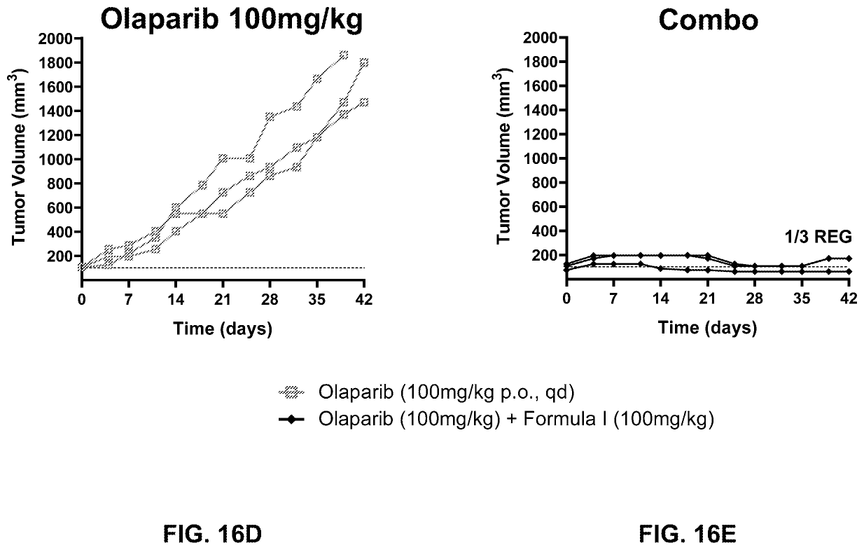

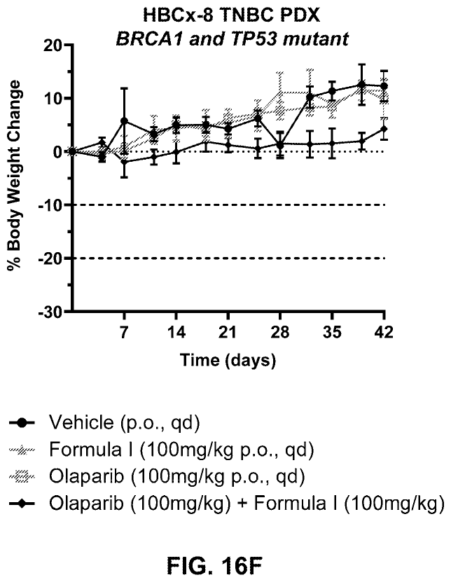

The data in FIGS. 16A-E show that, compared to equivalent doses of single agent Formula I or Olaparib, the combination treatment group showed enhanced anti-tumor activity in the HBCx-8 triple negative breast cancer BRCA1 mutant, TP53 mutant, HRD high, and RAD51 high patient-derived xenograft mouse model. The body weight data in FIG. 16F show that the combination was well tolerated.

### Example 17: Anti-Tumor Activity of Formula I Co-Crystal in Combination with the PARP Inhibitor, Olaparib, in the Breast HBCx-17 Patient-Derived Breast Xenograft Model in Nude Mice

Anti-tumor activity of the USP1 inhibitor, Formula I co-crystal in combination with Olaparib was evaluated in mice using a variety of patient-derived xenograft models, including the triple negative breast cancer HBCx-17 model. 6-9 week old female athymic nude mice from Envigo were anesthetized, and a 20 mm³ tumor fragment was placed subcutaneously via incision in the flank. When tumors reached a tumor volume ranging from approximately 60 to 200 mm³, mice were randomized into groups of 8-10 mice and were assigned into the following groups: control (vehicle), Formula I (100 mg/kg), Formula I (300 mg/kg), Olaparib (50 mg/kg), Olaparib (100 mg/kg), Formula I (100 mg/kg) and Olaparib (50 mg/kg) combination, or Formula I (100 mg/kg) and Olaparib (100 mg/kg) combination. Compound was administered via oral gavage once daily for 43 days (day 0 to 42). Body weight and tumor volume were measured twice per week. Tolerability was assessed by calculating body weight changes as percent (%) from body weight on day of treatment start (day 0). Tumor volume was calculated as mean and standard error of the mean for each treatment group. A tumor regression (REG) was defined as a tumor with a smaller volume of the last day of the study as compared to the first day of dosing, and a complete regression (CR) was defined as no palpable tumor at the end of the study.

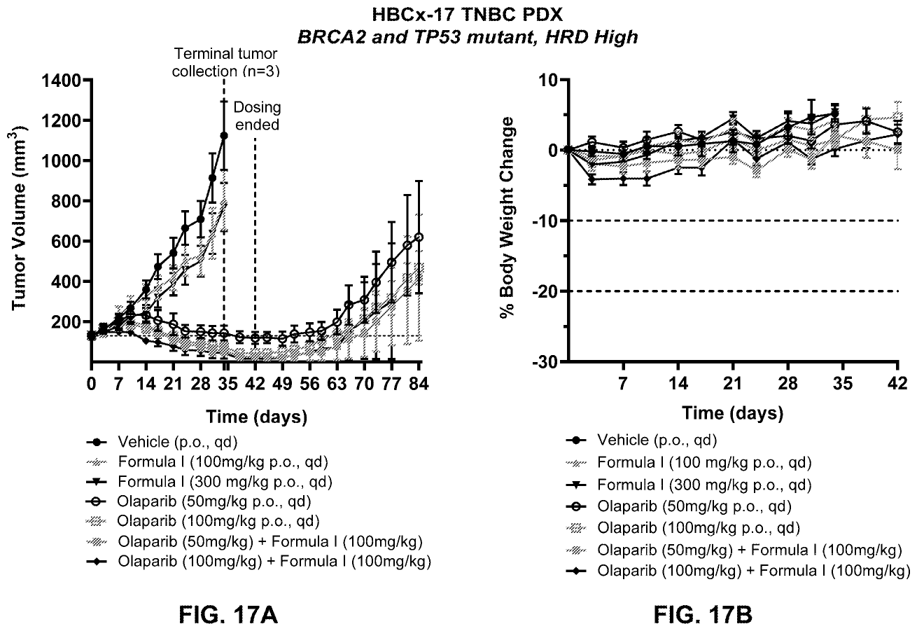

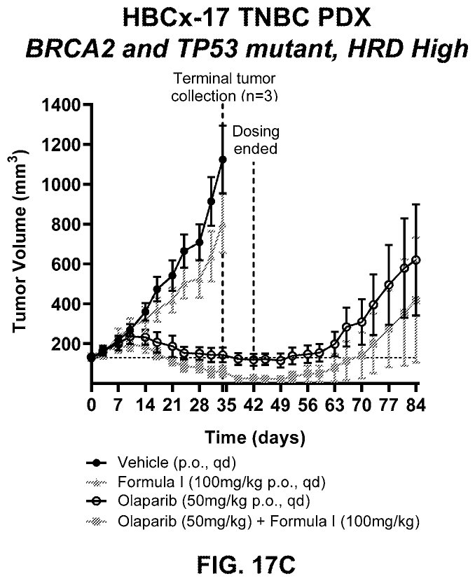

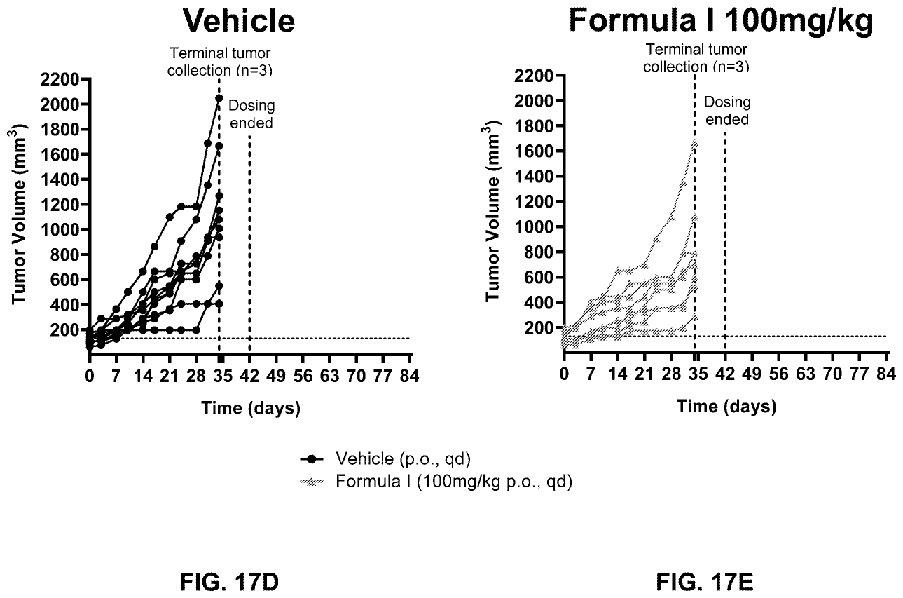

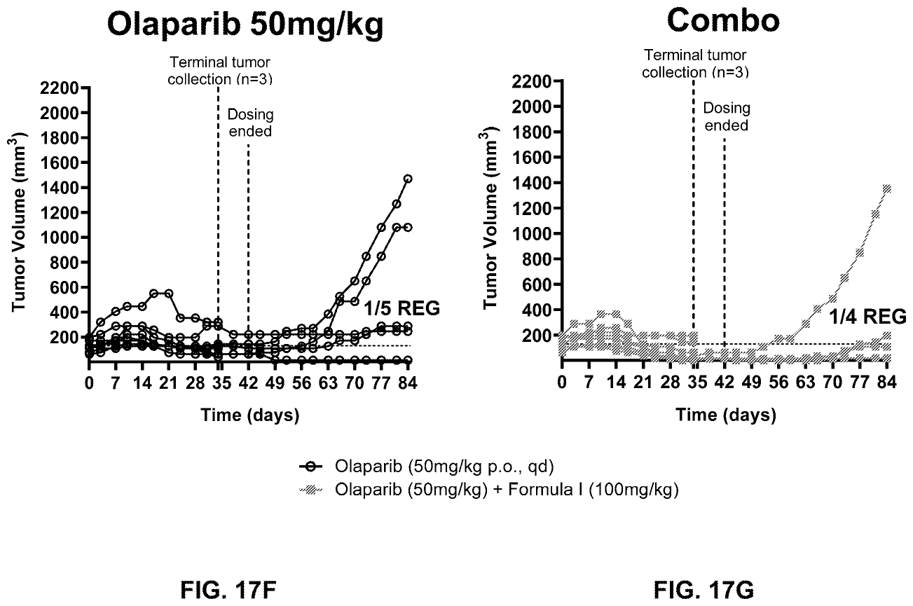

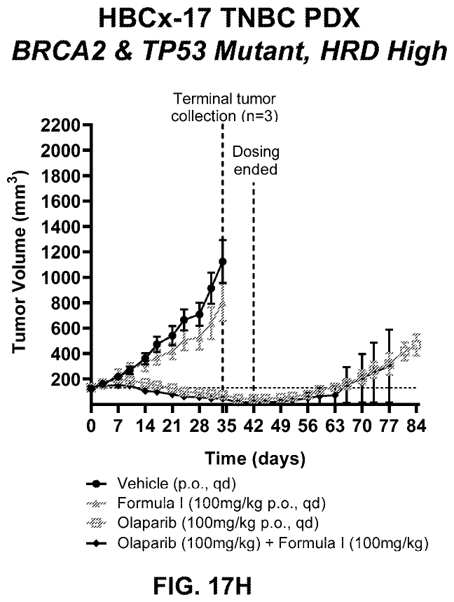

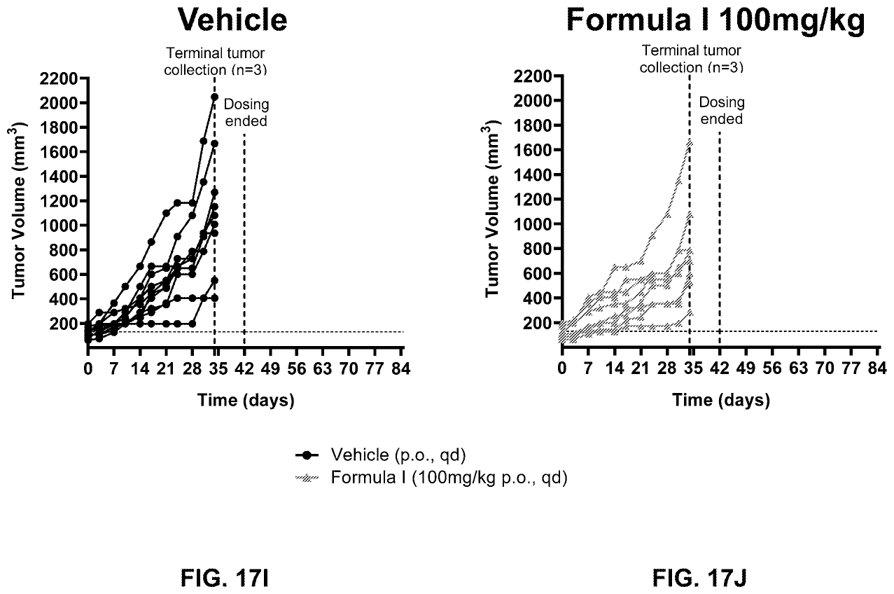

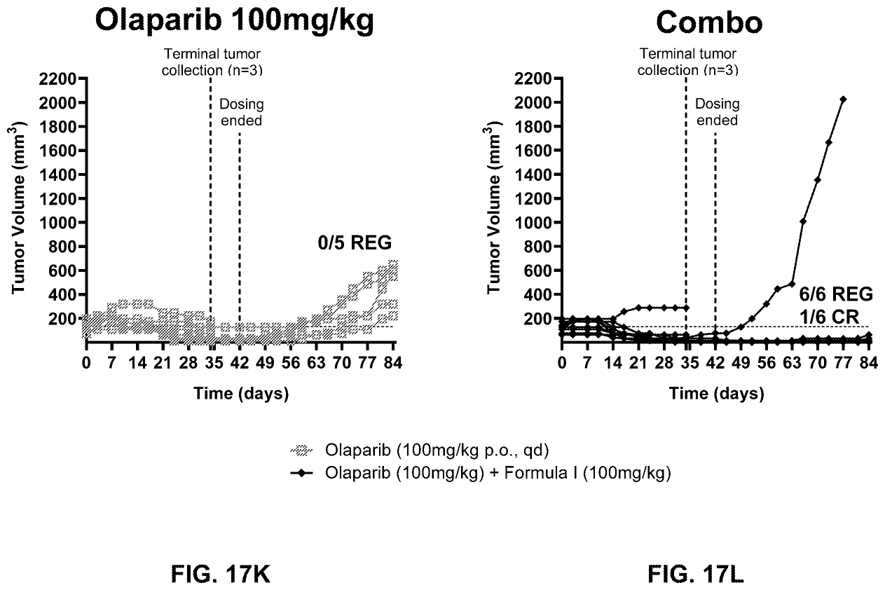

The data in FIGS. 17A and 17C-L show that, compared to the equivalent doses of single agents Formula I and Olaparib, both combination treatment groups showed enhanced anti-tumor activity in the HBCx-17 patient-derived xenograft mouse model. The body weight data in FIG. 17B show that the combinations were well tolerated.

### Example 18: Anti-Tumor Activity of Formula I Co-Crystal in Combination with the PARP Inhibitor, Olaparib, in the Ovarian CTG-0703 Patient-Derived Xenograft Model in Nude Mice

Anti-tumor activity of the USP1 inhibitor, Formula I co-crystal in combination with Olaparib was evaluated in mice using a variety of patient-derived xenograft models, including the serous ovarian carcinoma model CTG-0703. 6-8 week old female athymic nude mice from Envigo were anesthetized, and a 60 mm³ tumor fragment was placed subcutaneously via incision in the flank. When tumors reached a tumor volume ranging from approximately 110 to 230 mm³, mice were randomized into groups of 3 mice and were assigned into the following groups: control (vehicle), Formula I (100 mg/kg), Formula I (300 mg/kg), Olaparib (50 mg/kg), Olaparib (100 mg/kg), Formula I (100 mg/kg) and Olaparib (50 mg/kg) combination, or Formula I (100 mg/kg) and Olaparib (100 mg/kg) combination. Compound was administered via oral gavage once daily for 60 days (day 0 to 59). Body weight and tumor volume were measured twice per week. Tolerability was assessed by calculating body weight changes as percent (%) from body weight on day of treatment start (day 0). Tumor volume was calculated as mean and standard error of the mean for each treatment group. A tumor regression (REG) was defined as a tumor with a smaller volume of the last day of the study as compared to the first day of dosing, and a complete regression (CR) was defined as no palpable tumor at the end of the study.

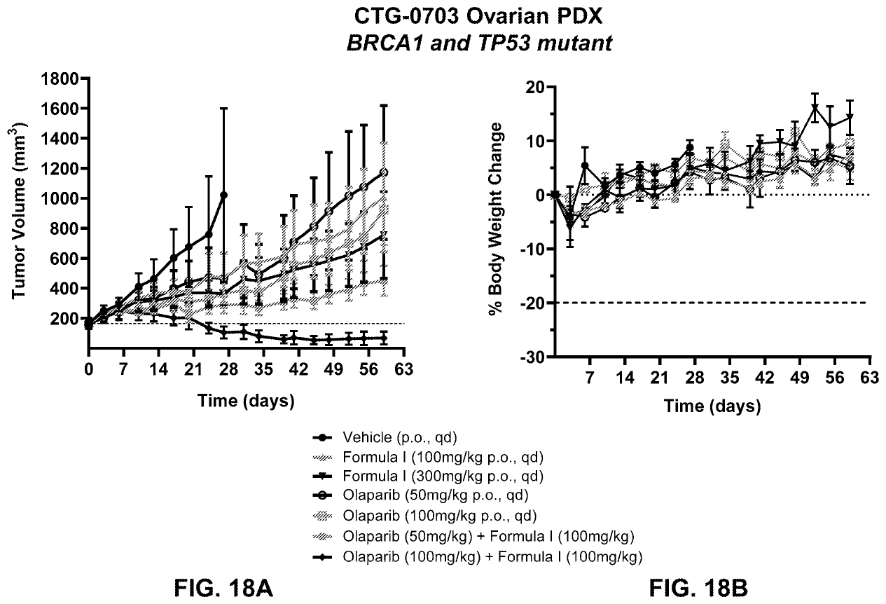

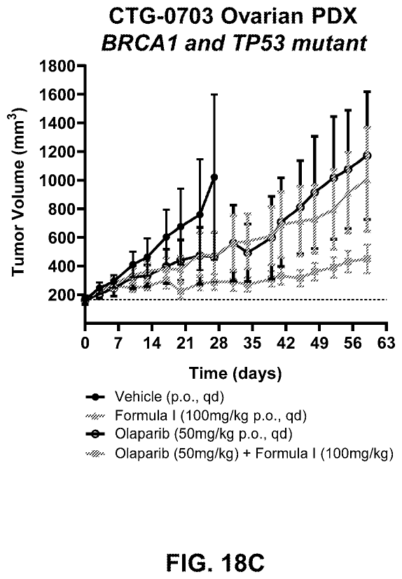

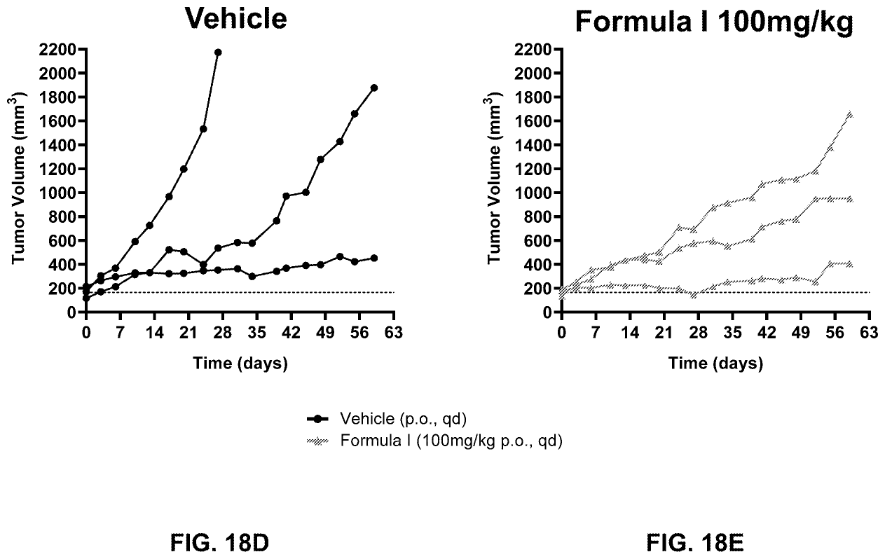

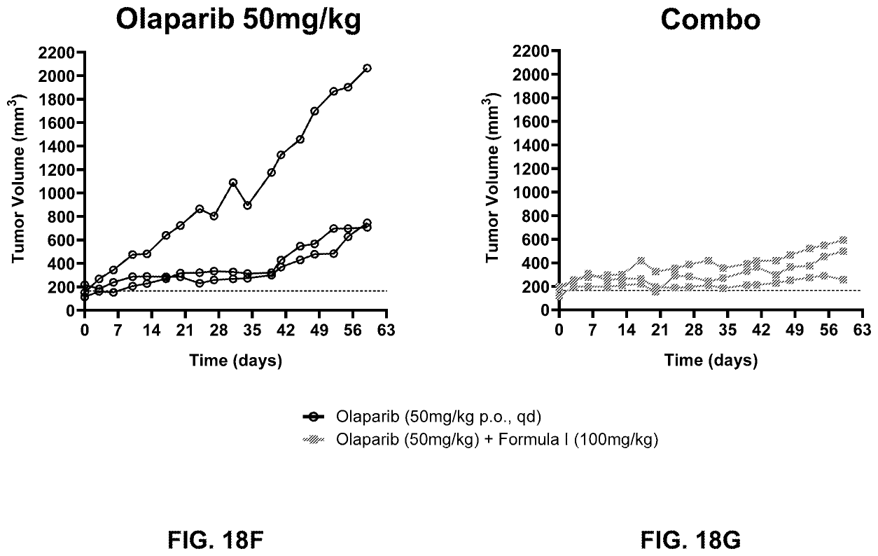

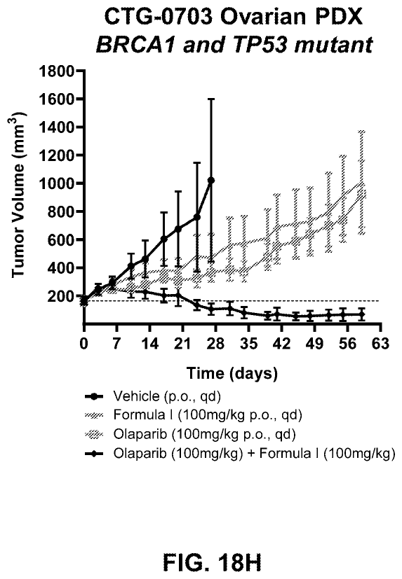

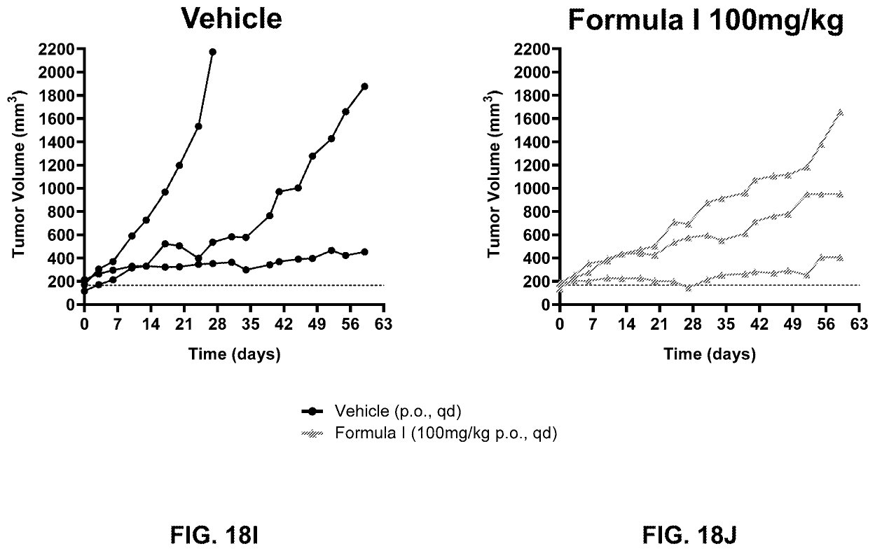

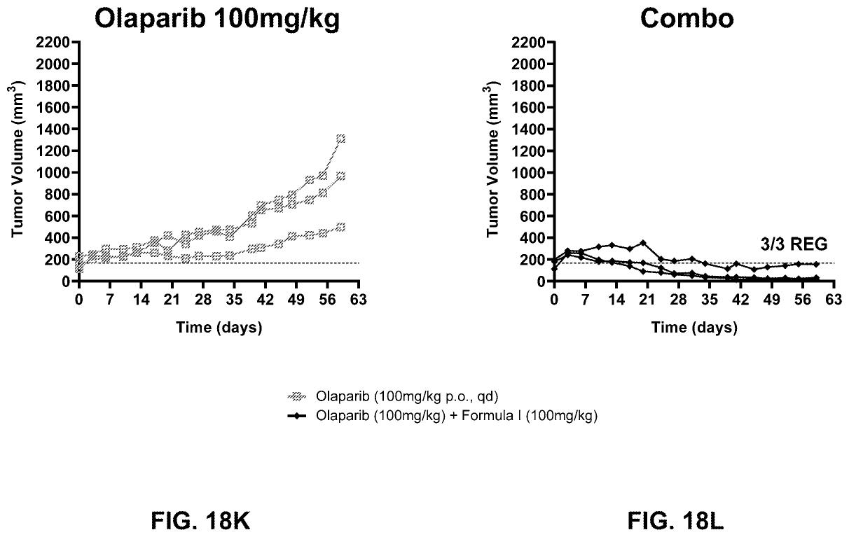

The data in FIGS. 18A and 18C-18L show that, compared to the equivalent doses of single agents Formula I and Olaparib, both combination treatment groups showed enhanced anti-tumor activity in the CTG-0703 patient-derived xenograft mouse model. The body weight data in FIG. 18B show that the combinations were well-tolerated.

Combination studies for three additional patient-derived ovarian carcinoma xenograft models, OV5308, OV5392, and OV0243, were carried out similarly to the above examples. No combination activity was observed in the OV5308, OV5392, and OV0243 models.

### Example 19: CRISPR Sensitization Screens

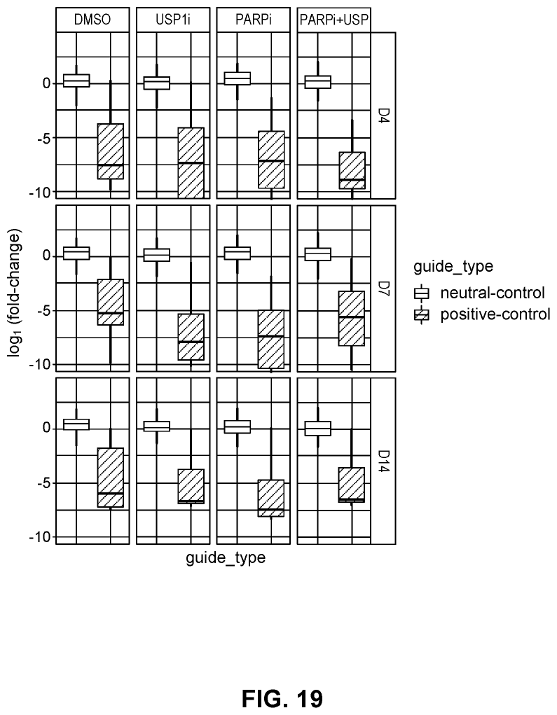

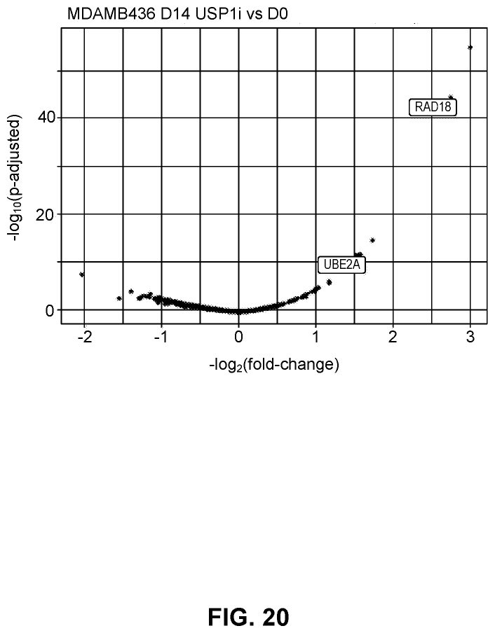

To perform CRISPR-Cas9 resistance screens, breast and ovarian cancer cell lines known to be sensitive to USP1 inhibition and/or PARP1 inhibitors were engineered to express Cas9 and were subsequently infected with lentivirus expressing guide RNAs targeting 1500 genes (20 sgRNAs per gene) involved in the DNA damage response and DNA repair. Infected cells were expanded for 10 days and split into different compound treatment arms: DMSO (negative control), 300 nM Formula I co-crystal, 300 nM Olaparib, and combination of 150 nM Formula I co-crystal plus 150 nM Olaparib. After 14 days of culture in the presence of drug, cells were harvested, genomic DNA was extracted, and Illumina Sequencing was used to determine guide representation. To determine the effect of a perturbation, the abundance of each sgRNA was compared to a reference sample, using both the plasmid library and the timepoint immediately prior to compound treatment initiation as references. For each guide in the library, the number of reads associated with that guide were counted, and the log-fold-change (logFC), defined as: logFC=log ((sample count+1)/(reference count+1)) was calculated. To ensure the magnitude of effect was comparable between experimental conditions, the scores associated with each guide were standardized by subtracting the median logFC for each sample from each guide, and dividing by the median absolute deviation, producing a Z-score for each guide. To aggregate guide-level scores to the gene-level, a per-gene “dropout score” was calculated for each gene targeted by the library by taking the median Z-score of all guides that target that gene. Differential dependencies, where CRISPR induced loss of gene function increases the cell's fitness in the presence of drug compared to DMSO treatment, were used to identify mechanism of drug resistance. For each gene a Fisher's Exact Test was used to test for an association between drug treatment and number of clones recovered, with false discoveries controlled using Benjamini Hochberg p-value adjustment. To assess screen quality, non-cutting neutral control guides were included, as well as positive control guides that target thousands of locations in the genome and robustly induce cell death. Control guides behaved as expected in screens of the breast cancer cell line MDA-MB-436, displaying a separation between positive and neutral control guides across all samples, and the majority of guides having no effect on fitness (FIG. 19). Importantly, gene knockout of previously described resistance mediators such as RAD18 and UBE2A were among the top enriched genes after Formula I co-crystal treatment in MDA-MB-436 (FIG. 20). In addition to these known resistance mechanisms, a number of new genes emerged as resistance mediators of Formula I co-crystal, which differ from the resistance hits after treatment with the PARP1 inhibitor Olaparib. Likewise, the same genes whose knockout led to resistance to Formula I co-crystal alone no longer led to resistance in combination with Olaparib, indicating non-overlapping resistance profiles.

Having now fully described this invention, it will be understood by those of ordinary skill in the art that the same can be performed within a wide and equivalent range of conditions, formulations, and other parameters without affecting the scope of the invention or any embodiment thereof.

Other aspects of the invention will be apparent to those skilled in the art from consideration of the specification and practice of the invention disclosed herein. It is intended that the specification and examples be considered as exemplary only, with a true scope and spirit of the invention being indicated by the following claims.

All patents and publications cited herein are fully incorporated by reference herein in their entirety.

What is claimed is:

1. A method of treating cancer in a subject who previously received treatment with a first poly ADP-ribose polymerase (PARP) inhibitor, the method comprising administering to the subject a ubiquitin-specific-processing protease (USP1) inhibitor and a second PARP inhibitor, wherein the first and the second PARP inhibitors are the same or different PARP inhibitors.

2. The method of claim 1, wherein the subject did not previously receive treatment with a USP1 inhibitor.

3. The method of claim 1 or 2, wherein the treatment with the first PARP inhibitor was interrupted or discontinued.

4. The method of claim 3, wherein the interruption is for at least one week, at least two weeks, at least three weeks, or at least four weeks.

5. The method of any one of claims 1-4, wherein the subject experienced unacceptable toxicity and/or unacceptable adverse reactions during treatment with the first PARP inhibitor.

6. The method of claim 1, wherein the unacceptable toxicity or adverse reaction was a hematological toxicity such as thrombocytopenia, anemia, or neutropenia, pneumonitis, dyspnea, fever, cough, wheezing, a radiological abnormality, hypertension, myelodysplastic syndrome/acute myeloid leukemia (MDS/AML), nausea, and/or fatigue.

7. The method of any one of claims 1-6, wherein during treatment with the first PARP inhibitor, the dose of the first PARP inhibitor was reduced.

8. The method of claim 7, wherein the dose of the first PARP inhibitor was reduced to one quarter, one third, one half, two thirds, or three quarters of the dose prior to the reduction.

9. The method of claim 7, wherein the first PARP inhibitor was olaparib and the dose prior to the reduction was 400 mg taken twice daily.

10. The method of claim 7 or 9, wherein the first PARP inhibitor was olaparib and the dose after the reduction was 200 mg taken twice daily or 100 mg taken twice daily.

11. The method of claim 7, wherein the first PARP inhibitor was niraparib and the dose prior to reduction was 300 mg taken once daily.

12. The method of claim 7 or 11, wherein the first PARP inhibitor was niraparib and the dose after the reduction was 200 mg taken once daily or 100 mg taken once daily.

13. The method of claim 7, wherein the first PARP inhibitor was talazoparib and the dose prior to reduction was 1 mg taken once daily.

14. The method of claim 7 or 13, wherein the first PARP inhibitor was talazoparib and the dose after the reduction was 0.75 mg taken once daily, 0.5 mg taken once daily, or 0.25 mg taken once daily.

15. The method of claim 7, wherein the first PARP inhibitor was rucaparib and the dose prior to reduction was 600 mg taken twice daily.

16. The method of claim 7 or 15, wherein the first PARP inhibitor was rucaparib and the dose after the reduction was 500 mg taken twice daily, 400 mg taken twice daily, or 300 mg taken twice daily.

17. The method of any one of claims 1-8, wherein the first PARP inhibitor was olaparib, niraparib, talazoparib, or rucaparib.

18. The method of any one of claims 1-17, wherein the second PARP inhibitor is olaparib, niraparib, talazoparib, or rucaparib.

19. The method of any one of claims 1-18, wherein the first PARP inhibitor and the second PARP inhibitor are the same PARP inhibitor.

20. The method of any one of claims 1-19, wherein the first PARP inhibitor and the second PARP inhibitor are different PARP inhibitors.

21. The method of any one of claims 1-20, wherein the dose of the second PARP inhibitor is reduced compared to the dose of first PARP inhibitor.

22. The method of any one of claims 1-21, wherein the USP1 inhibitor is a compound selected from the group consisting of 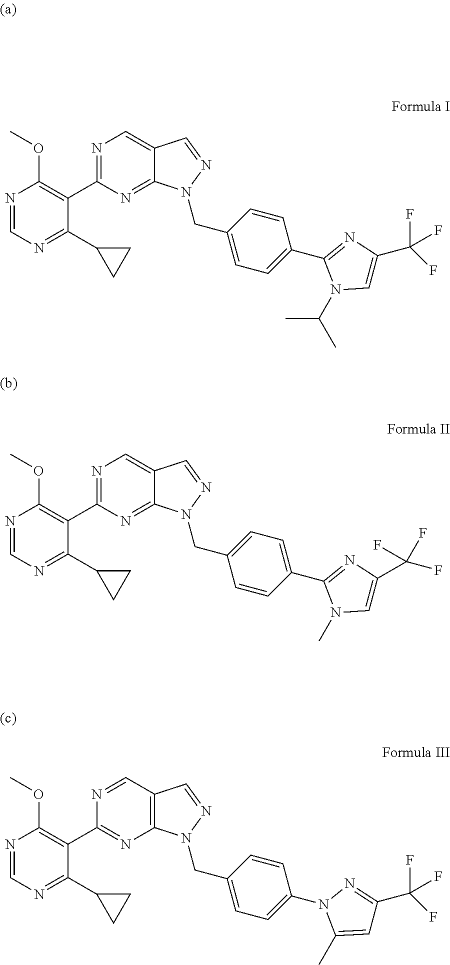 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1 ; COc1ncnc(C2CC2)c1-c1ncc2cnn(Cc3ccc(-n4nc(C(F)(F)F)cc4C)cc3)c2n1]
and pharmaceutically acceptable salts, hydrates, solvates, amorphous solids, or polymorphs thereof.

23. The method of any one of claims 1-22, wherein the USP1 inhibitor and the second PARP inhibitor are well tolerated.

24. The method of any one of claims 1-23, wherein the USP1 inhibitor decreases the exposure of the subject to the second PARP inhibitor.

25. The method of any one of claims 1-24, wherein the USP1 inhibitor and the second PARP inhibitor inhibit rebounding and/or regrowth of the cancer.

26. The method of any one of claims 1-25, wherein the USP1 inhibitor and the second PARP inhibitor are administered sequentially.

27. The method of any one of claims 1-26, wherein the USP1 inhibitor and the second PARP inhibitor are administered simultaneously.

28. The method of any one of claims 1-27, wherein the subject is human.

29. The method of any one of claims 1-28, wherein the cancer is selected from the group consisting of brain cancer, lung cancer, non-small cell lung cancer (NSCLC), colon cancer, bladder cancer, osteosarcoma, ovarian cancer, skin cancer, uterine cancer, peritoneal cancer, and endometrial cancer, and breast cancer.

30. The method of claim 29, wherein the cancer is breast cancer.

31. The method of claim 30, wherein the breast cancer is triple negative breast cancer (TNBC).

32. The method of any one of claims 1-31, wherein the cancer is a BRCA1 mutant cancer, a BRCA2 mutant cancer, or a BRCA1 mutant and BRCA2 mutant cancer.

33. The method of claim 29, wherein the cancer is ovarian cancer.

34. The method of claim 33, wherein the ovarian cancer is a BRCA1 mutant cancer, a BRCA2 mutant cancer, or a p53 mutant cancer.

35. The method of claim 33 or 34, wherein the ovarian cancer is a BRCA1 mutant cancer and a p53 mutant cancer.

36. The method of claim 33 or 34, wherein the ovarian cancer is a BRCA1 and BRCA2 mutant cancer.

37. The method of claim 33 or 34, wherein the ovarian cancer is a BRCA2 mutant cancer.

38. The method of any one of claims 1-28, wherein the cancer is selected from the group consisting of a hematological cancer and a lymphatic cancer.

39. The method of any one of claims 1-38, wherein the cancer comprises cells with elevated levels of RAD51.

40. The method of claim 39, wherein the elevated levels of RAD51 are elevated RAD51 protein levels.

41. The method of claim 39, wherein the elevated levels of RAD51 are elevated RAD51 protein foci levels.

42. The method of claim 39, wherein at least 10% of cells that are in the S/G2 phase of the cell cycle in a sample obtained from the cancer are RAD51-positive.

43. The method of claim 39, wherein the elevated levels of RAD51 are elevated RAD51 mRNA levels.

44. The method of any one of claims 39-43, wherein the elevated levels of RAD51 have been detected prior to the administration.

45. The method of claim 44, further comprising detecting RAD51 levels in a cancer sample obtained from the subject prior to the administration.

46. The method of any one of claims 1-45, wherein the cancer is selected from the group consisting of a DNA damage repair pathway deficient cancer, a homologous-recombination deficient cancer, a cancer comprising cancer cells with a mutation in a gene encoding p53, a cancer comprising cancer cells with a loss of function mutation in a gene encoding p53, and a cancer comprising cells with a mutation in the gene encoding ATM.

47. The method of any one of claims 1-46, wherein the cancer is a PARP inhibitor resistant or refractory cancer.

48. A method of treating cancer in a subject comprising administering to the subject a USP1 inhibitor, wherein the cancer comprises cancer cells with elevated levels of RAD51.

49. The method of claim 48, wherein the elevated levels of RAD51 have been detected prior to the administration.

50. The method of claim 49, further comprising detecting RAD51 levels in a cancer sample obtained from the subject.

51. The method of any one of claims 48-50, wherein the method further comprises administering to the subject a PARP inhibitor in combination with the USP1 inhibitor.

52. The method of claim 51, wherein the PARP inhibitor is olaparib, niraparib, talazoparib, or rucaparib.

53. A method of selecting a subject with cancer for treatment with a USP1 inhibitor, comprising detecting whether the cancer comprises cells with elevated levels of RAD51, wherein if the cancer comprises cells with elevated levels of RAD51, the subject is selected for treatment with a USP1 inhibitor.

54. An in vitro method for identifying a subject with cancer to be responsive to the treatment with a USP1 inhibitor, comprising detecting RAD51 levels in a cancer sample obtained from the subject, wherein elevated levels of RAD51 in the cancer sample are indicative for the patient to be responsive to the treatment with a USP1 inhibitor.

55. An in vitro use of at least one agent capable of specifically detecting RAD51, for identifying a subject with cancer to be responsive to the treatment with a USP1 inhibitor.

56. The method or use of any one of claims 53-55, wherein the treatment with a USP1 inhibitor further comprises treatment with a PARP inhibitor in combination with the USP1 inhibitor.

57. The method or use of claim 56, wherein the PARP inhibitor is olaparib, niraparib, talazoparib, or rucaparib.

58. The method or use of any one of claims 53-57, wherein the USP1 inhibitor is a compound selected from the group consisting of 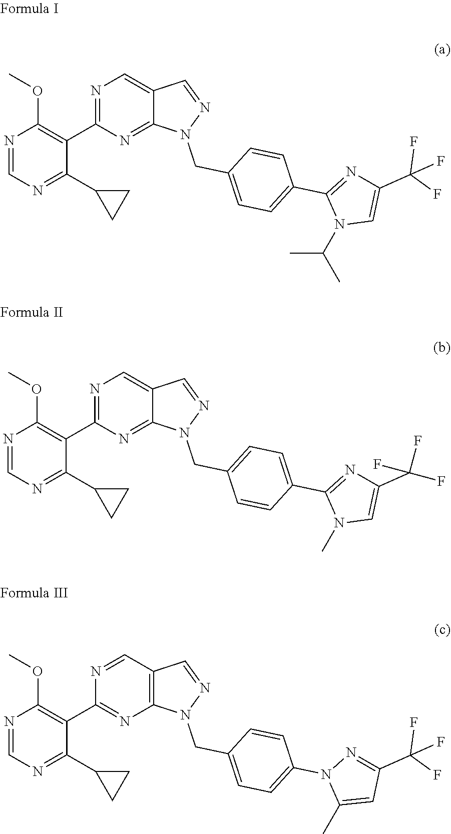 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1 ; COc1ncnc(C2CC2)c1-c1ncc2cnn(Cc3ccc(-n4nc(C(F)(F)F)cc4C)cc3)c2n1]
and pharmaceutically acceptable salts, hydrates, solvates, amorphous solids, or polymorphs thereof.

59. The method or use of any one of claims 48-58, wherein the subject is human.

60. The method or use of any one of claims 48-59, wherein the cancer is selected from the group consisting of brain cancer, lung cancer, non-small cell lung cancer (NSCLC), colon cancer, bladder cancer, osteosarcoma, ovarian cancer, skin cancer, uterine cancer, peritoneal cancer, and endometrial cancer, and breast cancer.

61. The method of claim 60, wherein the cancer is breast cancer.

62. The method of claim 61, wherein the breast cancer is triple negative breast cancer (TNBC).

63. The method of any one of claims 48-62, wherein the cancer is a BRCA1 mutant cancer, a BRCA2 mutant cancer, or a BRCA1 mutant and BRCA2 mutant cancer.

64. The method of claim 60, wherein the cancer is ovarian cancer.

65. The method of claim 64, wherein the ovarian cancer is a BRCA1 mutant cancer, a BRCA2 mutant cancer, or a p53 mutant cancer.

66. The method of claim 64 or 65, wherein the ovarian cancer is a BRCA1 mutant cancer and a p53 mutant cancer.

67. The method of claim 64 or 65, wherein the ovarian cancer is a BRCA1 and BRCA2 mutant cancer.

68. The method of claim 64 or 65, wherein the ovarian cancer is a BRCA2 mutant cancer.

69. A method of delaying, reducing, or preventing rebounding of a tumor in a subject comprising administering to the subject (i) a ubiquitin-specific-processing protease 1 (USP1) inhibitor and (ii) a poly ADP-ribose polymerase (PARP) inhibitor, or a pharmaceutically acceptable salt, hydrate, solvate, amorphous solid, or polymorph thereof,
wherein the USP1 inhibitor is a compound selected from the group consisting of 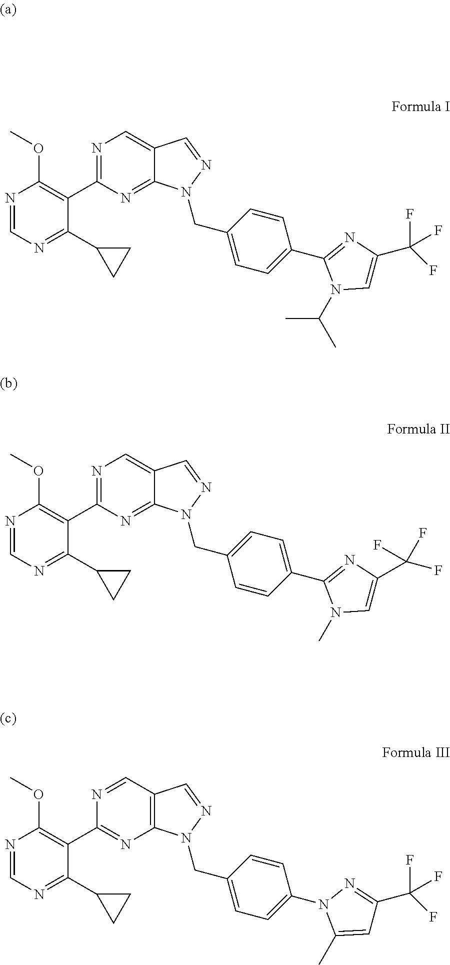 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1 ; COc1ncnc(C2CC2)c1-c1ncc2cnn(Cc3ccc(-n4nc(C(F)(F)F)cc4C)cc3)c2n1]
and pharmaceutically acceptable salts, hydrates, solvates, amorphous solids, or polymorphs thereof.

70. A method of treating cancer in a subject comprising administering to the subject (i) a ubiquitin-specific-processing protease 1 (USP1) inhibitor and (ii) a poly ADP-ribose polymerase (PARP) inhibitor, or a pharmaceutically acceptable salt, hydrate, solvate, amorphous solid, or polymorph thereof,
wherein the USP1 inhibitor is a compound selected from the group consisting of 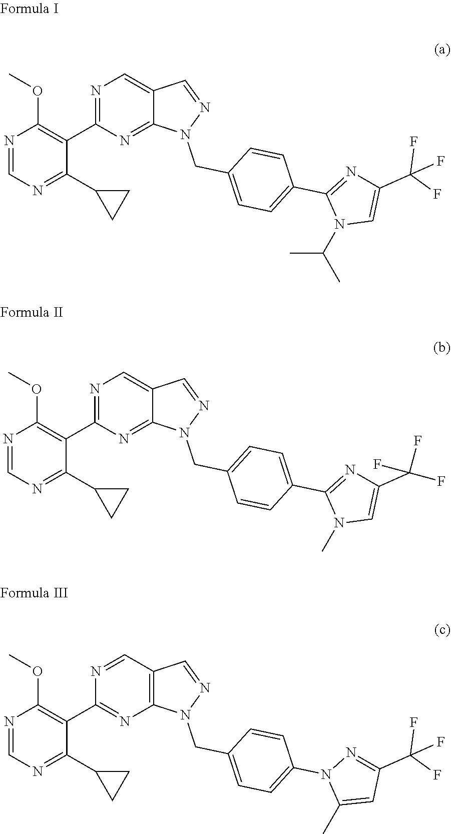 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1 ; COc1ncnc(C2CC2)c1-c1ncc2cnn(Cc3ccc(-n4nc(C(F)(F)F)cc4C)cc3)c2n1]
and pharmaceutically acceptable salts, hydrates, solvates, amorphous solids, or polymorphs thereof.

71. The method of claim 69 or 70, wherein said PARP inhibitor is selected from the group consisting of niraparib, olaparib and pharmaceutically acceptable salts, hydrates, solvates, amorphous solids, or polymorphs thereof.

72. The method of claim 71, wherein said PARP inhibitor is niraparib or a pharmaceutically acceptable salt, hydrate, solvate, amorphous solid, or polymorph thereof.

73. The method of claim 71, wherein said PARP inhibitor is olaparib or a pharmaceutically acceptable salt, hydrate, solvate, amorphous solid, or polymorph thereof.

74. The method of any one of claims 69 to 73, wherein the USP1 inhibitor is the compound of Formula I, or a pharmaceutically acceptable salt, hydrate, solvate, amorphous solid, or polymorph thereof.

75. The method of any one of claims 69 to 73, wherein the USP1 inhibitor is the compound of Formula II, or a pharmaceutically acceptable salt, hydrate, solvate, amorphous solid, or polymorph thereof.

76. The method of any one of claims 69 to 73, wherein the USP1 inhibitor is the compound of Formula III, or a pharmaceutically acceptable salt, hydrate, solvate, amorphous solid, or polymorph thereof.

77. The method of any one of claims 69 to 76, wherein the administration of the USP1 inhibitor, or said pharmaceutically acceptable salt, hydrate, solvate, amorphous solid, or polymorph thereof, and said PARP inhibitor, or said pharmaceutically acceptable salt, hydrate, solvate, amorphous solid, or polymorph thereof, provides a synergistic effect.

78. The method of any one of claims 70 to 77, wherein said USP1 inhibitor, or said pharmaceutically acceptable salt, hydrate, solvate, amorphous solid, or polymorph thereof, and said PARP inhibitor, or said pharmaceutically acceptable salt, hydrate, solvate, amorphous solid, or polymorph thereof, are administered in a therapeutically effective amount sufficient to produce one or more therapeutic effects selected from the group consisting of (i) reduction in tumor size, (ii) increase in tumor regression rate, and (iii) reduction or inhibition of tumor growth.

79. The method of any one of claims 69 to 78, wherein said USP1 inhibitor, or said pharmaceutically acceptable salt, hydrate, solvate, amorphous solid, or polymorph thereof, and said PARP inhibitor, or said pharmaceutically acceptable salt, hydrate, solvate, amorphous solid, or polymorph thereof, are administered in an amount effective to reduce the toxicity effects of a PARP inhibitor administered as a monotherapy.

80. The method of any one of claims 70 to 79, wherein said USP1 inhibitor, or said pharmaceutically acceptable salt, hydrate, solvate, amorphous solid, or polymorph thereof, and said PARP inhibitor, or said pharmaceutically acceptable salt, hydrate, solvate, amorphous solid, or polymorph thereof, delay, reduce, or prevent rebounding of a tumor.

81. The method of any one of claims 69 to 80, wherein the USP1 inhibitor, or said pharmaceutically acceptable salt, hydrate, solvate, amorphous solid, or polymorph thereof, and said PARP inhibitor, or said pharmaceutically acceptable salt, hydrate, solvate, amorphous solid, or polymorph thereof, are administered sequentially.

82. The method of any one of claims 69 to 80, wherein the USP1 inhibitor, or said pharmaceutically acceptable salt, hydrate, solvate, amorphous solid, or polymorph thereof, and said PARP inhibitor, or said pharmaceutically acceptable salt, hydrate, solvate, amorphous solid, or polymorph thereof, are administered simultaneously.

83. The method of any one of claims 69 to 82, wherein the USP1 inhibitor and/or the PARP inhibitor is administered at a dose that is not effective as a single agent.

84. The method of any one of claims 69 to 83, wherein the subject is a mammal, optionally, wherein the mammal is a human.

85. A combination composition comprising (i) a ubiquitin-specific-processing protease 1 (USP1) inhibitor and (ii) a poly ADP-ribose polymerase (PARP) inhibitor, or a pharmaceutically acceptable salt, hydrate, solvate, amorphous solid, or polymorph thereof, wherein the USP1 inhibitor is a compound selected from the group consisting of 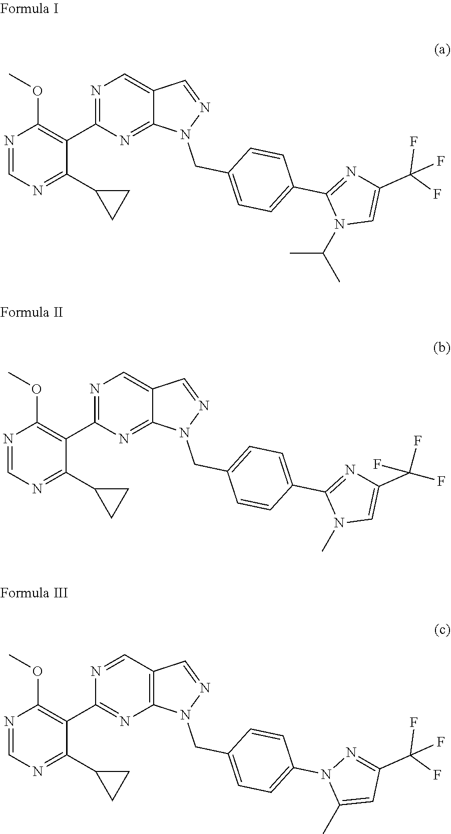 [SMILES: COc1ncnc(C2CC2)c1-c1ncc2cnn(Cc3ccc(-c4nc(C(F)(F)F)cn4C(C)C)cc3)c2n1 ; COc1ncnc(C2CC2)c1-c1ncc2cnn(Cc3ccc(-c4nc(C(F)(F)F)cn4C)cc3)c2n1 ; COc1ncnc(C2CC2)c1-c1ncc2cnn(Cc3ccc(-n4nc(C(F)(F)F)cc4C)cc3)c2n1]
and pharmaceutically acceptable salts, hydrates, solvates, amorphous solids, or polymorphs thereof.

86. The composition of claim 85, wherein said PARP inhibitor is selected from the group consisting of niraparib, olaparib and pharmaceutically acceptable salts, hydrates, solvates, amorphous solids, or polymorphs thereof.

87. The composition of claim 86, wherein said PARP inhibitor is niraparib or a pharmaceutically acceptable salt, hydrate, solvate, amorphous solid, or polymorph thereof.

88. The composition of claim 86, wherein said PARP inhibitor is olaparib or a pharmaceutically acceptable salt, hydrate, solvate, amorphous solid, or polymorph thereof.

89. The composition of any one of claims 85 to 88, wherein the USP1 inhibitor is the compound of Formula I, or a pharmaceutically acceptable salt, hydrate, solvate, amorphous solid, or polymorph thereof.

90. The composition of any one of claims 85 to 88, wherein the USP1 inhibitor is the compound of Formula II, or a pharmaceutically acceptable salt, hydrate, solvate, amorphous solid, or polymorph thereof.

91. The composition of any one of claims 85 to 88, wherein the USP1 inhibitor is the compound of Formula III, or a pharmaceutically acceptable salt, hydrate, solvate, amorphous solid, or polymorph thereof.

92. Use of the composition of any one of claims 85 to 91 for the manufacture of a medicament for treatment of cancer.

93. A pharmaceutical composition comprising the composition of any one of claims 85 to 91 and a pharmaceutically acceptable carrier.

94. The pharmaceutical composition of claim 93 for use in the treatment of cancer.

95. A kit comprising the composition of any one of claims 85 to 91, or the pharmaceutical composition of claim 93 or 94, and instructions for administering the combination to a subject having cancer.

96. The method of any one of claims 69 to 84, the use of claim 92, the pharmaceutical composition of claim 94, or the kit of claim 95, wherein the cancer is selected from the group consisting of a hematological cancer, a lymphatic cancer, a DNA damage repair pathway deficient cancer, a homologous-recombination deficient cancer, a cancer comprising cancer cells with a mutation in a gene encoding p53, and a cancer comprising cancer cells with a loss of function mutation in a gene encoding p53.

97. The method, use, pharmaceutical composition, or kit of any one of claims 69 to 84, 92, 94, 95, or 96, wherein the cancer is selected from the group consisting of brain cancer, lung cancer, non-small cell lung cancer (NSCLC), colon cancer, bladder cancer, osteosarcoma, ovarian cancer, skin cancer, uterine cancer, peritoneal cancer, and endometrial cancer, and breast cancer.

98. The method, use, pharmaceutical composition, or kit of any one of claims 69 to 84, 92, 94, 95, or 96, wherein the cancer is ovarian cancer or breast cancer.

99. The method, use, pharmaceutical composition, or kit of any one of claims 69 to 84, 92, 94, 95, or 96, wherein the cancer is ovarian cancer.

100. The method, use, pharmaceutical composition, or kit of any one of claims 69 to 84, 92, 94, 95, or 96, wherein the cancer is a breast cancer.

101. The method, use, pharmaceutical composition, or kit of any one of claims 69 to 84, 92, 94, 95, or 96, wherein the cancer is a triple negative breast cancer.

102. The method, use, pharmaceutical composition, or kit of any one of claims 69 to 84, 92, 94 to 101, wherein the cancer is a DNA damage repair pathway deficient cancer.

103. The method, use, pharmaceutical composition, or kit of claim 102, wherein the cancer is a homologous-recombination deficient cancer.

104. The method, use, pharmaceutical composition, or kit of any one of claims 69 to 84, 92, 94 to 103, wherein the cancer is a BRCA1 mutant cancer.

105. The method, use, pharmaceutical composition, or kit of any one of claims 69 to 84, 92, 94 to 103, wherein the cancer is a BRCA2 mutant cancer.

106. The method, use, pharmaceutical composition, or kit of any one of claims 69 to 84, 92, 94 to 103, wherein the cancer is a BRCA1 mutant cancer and a BRCA2 mutant cancer.

107. The method, use, pharmaceutical composition, or kit of any one of claims 69 to 84, 92, 94 to 106, wherein the cancer is a PARP inhibitor resistant or refractory cancer.

108. The method, use, pharmaceutical composition, or kit of any one of claims 69 to 84, 92, 94 to 107, wherein the cancer comprises cancer cells with a mutation in a gene encoding ATM.

109. The method, use, pharmaceutical composition, or kit of any one of claims 69 to 84, 92, 94 to 108, wherein the cancer comprises cells with elevated levels of RAD51.

110. The method, use, pharmaceutical composition, or kit of claim 109, wherein the elevated levels of RAD51 are elevated RAD51 protein levels.

111. The method, use, pharmaceutical composition, or kit of claim 109, wherein the elevated levels of RAD51 are elevated RAD51 protein foci levels.

112. The method, use, pharmaceutical composition, or kit of claim 109, wherein at least 10% of cells that are in the S/G2 phase of the cell cycle in a sample obtained from the cancer are RAD51-positive.

113. The method, use, pharmaceutical composition, or kit of claim 109, wherein the elevated levels of RAD51 are elevated RAD51 mRNA levels.

114. The method, use, pharmaceutical composition, or kit of any one of claims 109-113, wherein the elevated levels of RAD51 have been detected prior to the administration or the treatment.

115. The method or use of claim 114, further comprising detecting RAD51 levels in a cancer sample obtained from the subject prior to the administration or the treatment.

116. A method of treating a USP1 protein mediated disorder and/or a PARP protein mediated disorder comprising administering to a subject in need thereof a composition of any one of claims 85 to 91, or the pharmaceutical composition of claim 93 in an effective amount to treat the USP1 protein mediated disorder and/or the PARP protein mediated disorder.

117. A method of inhibiting a USP1 protein and/or a PARP protein comprising contacting a USP1 protein and/or a PARP protein with the composition of any one of claims 85 to 91, or the pharmaceutical composition of claim 93.

118. The method of claim 117, wherein the contacting occurs in vitro.

119. The method of claim 117, wherein the contacting occurs in vivo.
